SEQUENCE LISTING (176 sequences)

SEQ ID NO: 1 - protein, 8 aa, synthetic construct
  REGION 1..8: note = Synthetic peptide
GFTFSDYY

SEQ ID NO: 2 - protein, 8 aa, synthetic construct
  REGION 1..8: note = Synthetic peptide
ISTSGSTI

SEQ ID NO: 3 - protein, 15 aa, synthetic construct
  REGION 1..15: note = Synthetic peptide
CARDHYYSRGVIGYW

SEQ ID NO: 4 - protein, 6 aa, synthetic construct
  REGION 1..6: note = Synthetic peptide
QGISSW

SEQ ID NO: 5 - skipped

SEQ ID NO: 6 - protein, 11 aa, synthetic construct [sequence omitted]

SEQ ID NO: 7 - protein, 120 aa, synthetic construct
  REGION 1..120: note = Synthetic peptide
QVQLVESGGGLVKPGGSLRLSCAASGFTFSDYYMRWIRQAPGKGLEWVSHISTSGSTIYYADSVKGRFTISRDNAKNSLYLQMNSLRAEDTAVYYCARDHYYSRGVIGYWGQGTLVTVSS

SEQ ID NO: 8 - protein, 107 aa, synthetic construct
  REGION 1..107: note = Synthetic peptide
DIQMTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKLEIK

SEQ ID NO: 9 - protein, 450 aa, synthetic construct
  REGION 1..450: note = Synthetic peptide
QVQLVESGGGLVKPGGSLRLSCAASGFTFSDYYMRWIRQAPGKGLEWVSHISTSGSTIYYADSVKGRFTISRDNAKNSLYLQMNSLRAEDTAVYYCARDHYYSRGVIGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  REGION 1..214: note = Synthetic peptide
DIQMTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
  REGION 1..8: note = Synthetic peptide
GFTFSSYA

SEQ ID NO: 12 - protein, 8 aa, synthetic construct
  REGION 1..8: note = Synthetic peptide
ISNSGGTT

SEQ ID NO: 13 - protein, 15 aa, synthetic construct
  REGION 1..15: note = Synthetic peptide
CAKAHYYARGYFDYW

SEQ ID NO: 14 - protein, 6 aa, synthetic construct
  REGION 1..6: note = Synthetic peptide
QGISSW

SEQ ID NO: 15 - skipped

SEQ ID NO: 16 - protein, 11 aa, synthetic construct [sequence omitted]

SEQ ID NO: 17 - protein, 120 aa, synthetic construct
  REGION 1..120: note = Synthetic peptide
EVQLVESGGGLVQPGGSLRLSCAASGFTFSSYAMNWVRQAPGKGLEWVSTISNSGGTTYYADSVKGRFTISRDNSKDTLYLQMNSLRAEDTAVYYCAKAHYYARGYFDYWGQGTLVTVSS

SEQ ID NO: 18 - protein, 107 aa, synthetic construct
  REGION 1..107: note = Synthetic peptide
DIQMTQSPSSVSASVGDRVTITCRASQGISSWLAWYQQKSGKAPKLLIYAASSLQIGVPSRFSGSGSGTDFTLTISSLQPEDFATYYCQQANSFPYTFGQGTKLEIK

SEQ ID NO: 19 - protein, 450 aa, synthetic construct
  REGION 1..450: note = Synthetic peptide
EVQLVESGGGLVQPGGSLRLSCAASGFTFSSYAMNWVRQAPGKGLEWVSTISNSGGTTYYADSVKGRFTISRDNSKDTLYLQMNSLRAEDTAVYYCAKAHYYARGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
  REGION 1..214: note = Synthetic peptide
DIQMTQSPSSVSASVGDRVTITCRASQGISSWLAWYQQKSGKAPKLLIYAASSLQIGVPSRFSGSGSGTDFTLTISSLQPEDFATYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
  REGION 1..5: note = Synthetic peptide
NSGMH

SEQ ID NO: 22 - protein, 17 aa, synthetic construct
  REGION 1..17: note = Synthetic peptide
VIWYDGSKRYYADSVKG

SEQ ID NO: 23 - protein, 4 aa, synthetic construct
  REGION 1..4: note = Synthetic peptide
NDDY

SEQ ID NO: 24 - protein, 11 aa, synthetic construct
  REGION 1..11: note = Synthetic peptide
RASQSVSSYLA

SEQ ID NO: 25 - protein, 7 aa, synthetic construct
  REGION 1..7: note = Synthetic peptide
DASNRAT

SEQ ID NO: 26 - protein, 9 aa, synthetic construct
  REGION 1..9: note = Synthetic peptide
QQSSNWPRT

SEQ ID NO: 27 - protein, 113 aa, synthetic construct [sequence omitted]

SEQ ID NO: 28 - protein, 107 aa, synthetic construct [sequence omitted]

SEQ ID NO: 29 - protein, 440 aa, synthetic construct
  REGION 1..440: note = Synthetic peptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 214 aa, synthetic construct
  REGION 1..214: note = Synthetic peptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 5 aa, synthetic construct
  REGION 1..5: note = Synthetic peptide
NYYMY

SEQ ID NO: 32 - protein, 17 aa, synthetic construct
  REGION 1..17: note = Synthetic peptide
GINPSNGGTNFNEKFKN

SEQ ID NO: 33 - protein, 11 aa, synthetic construct
  REGION 1..11: note = Synthetic peptide
RDYRFDMGFDY

SEQ ID NO: 34 - protein, 15 aa, synthetic construct
  REGION 1..15: note = Synthetic peptide
RASKGVSTSGYSYLH

SEQ ID NO: 35 - protein, 7 aa, synthetic construct
  REGION 1..7: note = Synthetic peptide
LASYLES

SEQ ID NO: 36 - protein, 9 aa, synthetic construct
  REGION 1..9: note = Synthetic peptide
QHSRDLPLT

SEQ ID NO: 37 - protein, 120 aa, synthetic construct [sequence omitted]

SEQ ID NO: 38 - protein, 111 aa, synthetic construct [sequence omitted]

SEQ ID NO: 39 - protein, 447 aa, synthetic construct
  REGION 1..447: note = Synthetic peptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 218 aa, synthetic construct
  REGION 1..218: note = Synthetic peptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 8 aa, synthetic construct
  REGION 1..8: note = Synthetic peptide
GFTFSNFG

SEQ ID NO: 42 - protein, 8 aa, synthetic construct
  REGION 1..8: note = Synthetic peptide
ISGGGRDT

SEQ ID NO: 43 - protein, 10 aa, synthetic construct
  REGION 1..10: note = Synthetic peptide
VKWGNIYFDY

SEQ ID NO: 44 - protein, 6 aa, synthetic construct
  REGION 1..6: note = Synthetic peptide
LSINTF

SEQ ID NO: 45 - skipped

SEQ ID NO: 46 - protein, 9 aa, synthetic construct
  REGION 1..9: note = Synthetic peptide
QQSSNTPFT

SEQ ID NO: 47 - protein, 117 aa, synthetic construct [sequence omitted]

SEQ ID NO: 48 - protein, 107 aa, synthetic construct [sequence omitted]

SEQ ID NO: 49 - protein, 444 aa, synthetic construct
  REGION 1..444: note = Synthetic peptide
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, synthetic construct
  REGION 1..214: note = Synthetic peptide
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 10 aa, synthetic construct
  REGION 1..10: note = Synthetic peptide
GFTFSSYDMS

SEQ ID NO: 52 - protein, 10 aa, synthetic construct
  REGION 1..10: note = Synthetic peptide
TISGGGSYTY

SEQ ID NO: 53 - protein, 7 aa, synthetic construct
  REGION 1..7: note = Synthetic peptide
PYYAMDY

SEQ ID NO: 54 - protein, 11 aa, synthetic construct
  REGION 1..11: note = Synthetic peptide
KASQDVGTAVA

SEQ ID NO: 55 - protein, 7 aa, synthetic construct
  REGION 1..7: note = Synthetic peptide
WASTLHT

SEQ ID NO: 56 - protein, 9 aa, synthetic construct
  REGION 1..9: note = Synthetic peptide
QHYSSYPWT

SEQ ID NO: 57 - protein, 116 aa, synthetic construct
  REGION 1..116: note = Synthetic peptide
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 58 - protein, 107 aa, synthetic construct
  REGION 1..107: note = Synthetic peptide
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 59 - protein, 443 aa, synthetic construct
  REGION 1..443: note = Synthetic peptide
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 214 aa, synthetic construct
  REGION 1..214: note = Synthetic peptide
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 8 aa, synthetic construct
  REGION 1..8: note = Synthetic peptide
GFTFTRYD

SEQ ID NO: 62 - protein, 8 aa, synthetic construct
  REGION 1..8: note = Synthetic peptide
IGDSNKMT

SEQ ID NO: 63 - protein, 19 aa, synthetic construct
  REGION 1..19: note = Synthetic peptide
CAKGSCIACWDEAGRIDAW

SEQ ID NO: 64 - protein, 8 aa, synthetic construct
  REGION 1..8: note = Synthetic peptide
GSYDGSSY

SEQ ID NO: 65 - skipped

SEQ ID NO: 66 - protein, 17 aa, synthetic construct
  REGION 1..17: note = Synthetic peptide
CGSYDRPETNSDYVGMF

SEQ ID NO: 67 - protein, 124 aa, synthetic construct
  REGION 1..124: note = Synthetic peptide
EVQLLESGGGLVQPGGSLRLSCAASGFTFTRYDMVWVRQAPGKGLEWVAGIGDSNKMTRYAPAVKGRATISRDNSKNTLYLQMNSLRAEDTAVYYCAKGSCIACWDEAGRIDAWGQGTLVTVSS

SEQ ID NO: 68 - protein, 114 aa, synthetic construct
  REGION 1..114: note = Synthetic peptide
SYELTQDPAVSVALGQTVRITCSGGGSYDGSSYYGWYQQKPGQAPVTVIYNNNNRPSDIPDRFSGSSSGNTASLTITGAQAEDEADYYCGSYDRPETNSDYVGMFGSGTKVTVL

SEQ ID NO: 69 - protein, 454 aa, synthetic construct
  REGION 1..454: note = Synthetic peptide
EVQLLESGGGLVQPGGSLRLSCAASGFTFTRYDMVWVRQAPGKGLEWVAGIGDSNKMTRYAPAVKGRATISRDNSKNTLYLQMNSLRAEDTAVYYCAKGSCIACWDEAGRIDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 220 aa, synthetic construct
  REGION 1..220: note = Synthetic peptide
SYELTQDPAVSVALGQTVRITCSGGGSYDGSSYYGWYQQKPGQAPVTVIYNNNNRPSDIPDRFSGSSSGNTASLTITGAQAEDEADYYCGSYDRPETNSDYVGMFGSGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 71 - protein, 5 aa, synthetic construct
  REGION 1..5: note = Synthetic peptide
SYWMN

SEQ ID NO: 72 - protein, 17 aa, synthetic construct
  REGION 1..17: note = Synthetic peptide
VIHPSDSETWLDQKFKD

SEQ ID NO: 73 - protein, 10 aa, synthetic construct
  REGION 1..10: note = Synthetic peptide
EHYGTSPFAY

SEQ ID NO: 74 - protein, 15 aa, synthetic construct
  REGION 1..15: note = Synthetic peptide
RASESVDNYGMSFMN

SEQ ID NO: 75 - protein, 7 aa, synthetic construct
  REGION 1..7: note = Synthetic peptide
AASNQGS

SEQ ID NO: 76 - protein, 9 aa, synthetic construct
  REGION 1..9: note = Synthetic peptide
QQSKEVPYT

SEQ ID NO: 77 - protein, 119 aa, synthetic construct
  REGION 1..119: note = Synthetic peptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 78 - protein, 111 aa, synthetic construct
  REGION 1..111: note = Synthetic peptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 79 - protein, 445 aa, synthetic construct
  REGION 1..445: note = Synthetic peptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 218 aa, synthetic construct
  REGION 1..218: note = Synthetic peptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 10 aa, synthetic construct
  REGION 1..10: note = Synthetic peptide
GFTFSDSWIH

SEQ ID NO: 82 - protein, 18 aa, synthetic construct
  REGION 1..18: note = Synthetic peptide
AWISPYGGSTYYADSVKG

SEQ ID NO: 83 - protein, 9 aa, synthetic construct
  REGION 1..9: note = Synthetic peptide
RHWPGGFDY

SEQ ID NO: 84 - protein, 11 aa, synthetic construct
  REGION 1..11: note = Synthetic peptide
RASQDVSTAVA

SEQ ID NO: 85 - protein, 7 aa, synthetic construct
  REGION 1..7: note = Synthetic peptide
SASFLYS

SEQ ID NO: 86 - protein, 9 aa, synthetic construct
  REGION 1..9: note = Synthetic peptide
QQYLYHPAT

SEQ ID NO: 87 - protein, 118 aa, synthetic construct [sequence omitted]

SEQ ID NO: 88 - protein, 107 aa, synthetic construct [sequence omitted]

SEQ ID NO: 89 - protein, 448 aa, synthetic construct
  REGION 1..448: note = Synthetic peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct
  REGION 1..214: note = Synthetic peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 5 aa, synthetic construct
  REGION 1..5: note = Synthetic peptide
SYIMM

SEQ ID NO: 92 - protein, 17 aa, synthetic construct
  REGION 1..17: note = Synthetic peptide
SIYPSGGITFYADTVKG

SEQ ID NO: 93 - protein, 11 aa, synthetic construct
  REGION 1..11: note = Synthetic peptide
IKLGTVTTVDY

SEQ ID NO: 94 - protein, 16 aa, synthetic construct
  REGION 1..16: note = Synthetic peptide
SCTGTSSDVGGYNYVS

SEQ ID NO: 95 - protein, 7 aa, synthetic construct
  REGION 1..7: note = Synthetic peptide
DVSNRPS

SEQ ID NO: 96 - protein, 10 aa, synthetic construct
  REGION 1..10: note = Synthetic peptide
SSYTSSSTRV

SEQ ID NO: 97 - protein, 120 aa, synthetic construct
  REGION 1..120: note = Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 98 - protein, 110 aa, synthetic construct
  REGION 1..110: note = Synthetic peptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 99 - protein, 450 aa, synthetic construct
  REGION 1..450: note = Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 216 aa, synthetic construct
  REGION 1..216: note = Synthetic peptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 101 - protein, 10 aa, synthetic construct
  REGION 1..10: note = Synthetic peptide
GFTFSRYWMS

SEQ ID NO: 102 - protein, 17 aa, synthetic construct
  REGION 1..17: note = Synthetic peptide
NIKQDGSEKYYVDSVKG

SEQ ID NO: 103 - protein, 12 aa, synthetic construct
  REGION 1..12: note = Synthetic peptide
EGGWFGELAFDY

SEQ ID NO: 104 - protein, 12 aa, synthetic construct
  REGION 1..12: note = Synthetic peptide
RASQRVSSSYLA

SEQ ID NO: 105 - protein, 7 aa, synthetic construct
  REGION 1..7: note = Synthetic peptide
DASSRAT

SEQ ID NO: 106 - protein, 9 aa, synthetic construct
  REGION 1..9: note = Synthetic peptide
QQYGSLPWT

SEQ ID NO: 107 - protein, 121 aa, synthetic construct
  REGION 1..121: note = Synthetic peptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 108 - protein, 108 aa, synthetic construct
  REGION 1..108: note = Synthetic peptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 109 - protein, 451 aa, synthetic construct
  REGION 1..451: note = Synthetic peptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 215 aa, synthetic construct
  REGION 1..215: note = Synthetic peptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 5 aa, synthetic construct
  REGION 1..5: note = Synthetic peptide
NYGVH

SEQ ID NO: 112 - protein, 16 aa, synthetic construct
  REGION 1..16: note = Synthetic peptide
VIWSGGNTDYNTPFTS

SEQ ID NO: 113 - protein, 11 aa, synthetic construct
  REGION 1..11: note = Synthetic peptide
ALTYYDYEFAY

SEQ ID NO: 114 - protein, 11 aa, synthetic construct
  REGION 1..11: note = Synthetic peptide
RASQSIGTNIH

SEQ ID NO: 115 - protein, 7 aa, synthetic construct
  REGION 1..7: note = Synthetic peptide
YASESIS

SEQ ID NO: 116 - protein, 9 aa, synthetic construct
  REGION 1..9: note = Synthetic peptide
QQNNNWPTT

SEQ ID NO: 117 - protein, 119 aa, synthetic construct
  REGION 1..119: note = Synthetic peptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 118 - protein, 107 aa, synthetic construct
  REGION 1..107: note = Synthetic peptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 119 - protein, 449 aa, synthetic construct
  REGION 1..449: note = Synthetic peptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 214 aa, synthetic construct
  REGION 1..214: note = Synthetic peptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 8 aa, synthetic construct
  REGION 1..8: note = Synthetic peptide
SGDYYWTW

SEQ ID NO: 122 - protein, 17 aa, synthetic construct
  REGION 1..17: note = Synthetic peptide
HIYYSGNTNYNPSLKSR

SEQ ID NO: 123 - protein, 9 aa, synthetic construct
  REGION 1..9: note = Synthetic peptide
DRVTGAFDI

SEQ ID NO: 124 - protein, 10 aa, synthetic construct
  REGION 1..10: note = Synthetic peptide
ASQDISNYLN

SEQ ID NO: 125 - protein, 7 aa, synthetic construct
  REGION 1..7: note = Synthetic peptide
DASNLET

SEQ ID NO: 126 - protein, 9 aa, synthetic construct
  REGION 1..9: note = Synthetic peptide
QHFDHLPLA

SEQ ID NO: 127 - protein, 119 aa, synthetic construct [sequence omitted]

SEQ ID NO: 128 - protein, 107 aa, synthetic construct [sequence omitted]

SEQ ID NO: 129 - protein, 445 aa, synthetic construct
  REGION 1..445: note = Synthetic peptide
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 130 - protein, 214 aa, synthetic construct
  REGION 1..214: note = Synthetic peptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 8 aa, synthetic construct
  REGION 1..8: note = Synthetic peptide
GYTFTSYW

SEQ ID NO: 132 - protein, 8 aa, synthetic construct
  REGION 1..8: note = Synthetic peptide
IYPGSRST

SEQ ID NO: 133 - protein, 16 aa, synthetic construct
  REGION 1..16: note = Synthetic peptide
TRNGDYYVSSGDAMDY

SEQ ID NO: 134 - protein, 6 aa, synthetic construct
  REGION 1..6: note = Synthetic peptide
QDIGNY

SEQ ID NO: 135 - skipped

SEQ ID NO: 136 - protein, 9 aa, synthetic construct
  REGION 1..9: note = Synthetic peptide
QHYNTVPPT

SEQ ID NO: 137 - protein, 123 aa, synthetic construct
  REGION 1..123: note = Synthetic peptide
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSS

SEQ ID NO: 138 - protein, 107 aa, synthetic construct
  REGION 1..107: note = Synthetic peptide
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIK

SEQ ID NO: 139 - protein, 452 aa, synthetic construct
  REGION 1..452: note = Synthetic peptide
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 214 aa, synthetic construct
  REGION 1..214: note = Synthetic peptide
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 8 aa, synthetic construct
  REGION 1..8: note = Synthetic peptide
GYTFTSHW

SEQ ID NO: 142 - protein, 8 aa, synthetic construct
  REGION 1..8: note = Synthetic peptide
INPSSGRN

SEQ ID NO: 143 - protein, 12 aa, synthetic construct
  REGION 1..12: note = Synthetic peptide
VRYYGYDEAMDY

SEQ ID NO: 144 - protein, 11 aa, synthetic construct
  REGION 1..11: note = Synthetic peptide
KSLLHSNGITY

SEQ ID NO: 145 - skipped

SEQ ID NO: 146 - protein, 9 aa, synthetic construct
  REGION 1..9: note = Synthetic peptide
AQNLELPYT

SEQ ID NO: 147 - protein, 119 aa, synthetic construct
  REGION 1..119: note = Synthetic peptide
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSS

SEQ ID NO: 148 - protein, 112 aa, synthetic construct
  REGION 1..112: note = Synthetic peptide
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 149 - protein, 448 aa, synthetic construct
  REGION 1..448: note = Synthetic peptide
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 219 aa, synthetic construct
  REGION 1..219: note = Synthetic peptide
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 9 aa, synthetic construct
  REGION 1..9: note = Synthetic peptide
GFNIKDTYI

SEQ ID NO: 152 - protein, 4 aa, synthetic construct
  REGION 1..4: note = Synthetic peptide
YPTN

SEQ ID NO: 153 - protein, 9 aa, synthetic construct
  REGION 1..9: note = Synthetic peptide
GGDGFYAMD

SEQ ID NO: 154 - protein, 7 aa, synthetic construct
  REGION 1..7: note = Synthetic peptide
QDVNTAV

SEQ ID NO: 155 - skipped

SEQ ID NO: 156 - protein, 6 aa, synthetic construct
  REGION 1..6: note = Synthetic peptide
HYTTPP

SEQ ID NO: 157 - protein, 120 aa, synthetic construct [sequence omitted]

SEQ ID NO: 158 - protein, 107 aa, synthetic construct [sequence omitted]

SEQ ID NO: 159 - protein, 449 aa, synthetic construct
  REGION 1..449: note = Synthetic peptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 214 aa, synthetic construct
  REGION 1..214: note = Synthetic peptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 5 aa, synthetic construct
  REGION 1..5: note = Synthetic peptide
DTYIH

SEQ ID NO: 162 - protein, 17 aa, synthetic construct
  REGION 1..17: note = Synthetic peptide
RIYPTNGYTRYDPKFQD

SEQ ID NO: 163 - protein, 11 aa, synthetic construct
  REGION 1..11: note = Synthetic peptide
WGGDGFYAMDY

SEQ ID NO: 164 - protein, 11 aa, synthetic construct
  REGION 1..11: note = Synthetic peptide
KASQDVNTAVA

SEQ ID NO: 165 - protein, 7 aa, synthetic construct
  REGION 1..7: note = Synthetic peptide
SASFRYT

SEQ ID NO: 166 - protein, 9 aa, synthetic construct
  REGION 1..9: note = Synthetic peptide
QQHYTTPPT

SEQ ID NO: 167 - protein, 120 aa, synthetic construct
  REGION 1..120: note = Synthetic peptide
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 168 - protein, 107 aa, synthetic construct
  REGION 1..107: note = Synthetic peptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 169 - protein, 450 aa, synthetic construct
  REGION 1..450: note = Synthetic peptide
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 170 - protein, 214 aa, synthetic construct
  REGION 1..214: note = Synthetic peptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 233 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 172 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 173 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 174 - protein, 1210 aa, Homo sapiens [sequence omitted]

SEQ ID NO: 175 - protein, 1255 aa, Homo sapiens [sequence omitted]

SEQ ID NO: 176 - protein, 15 aa, synthetic construct
  REGION 1..15: note = Synthetic peptide
GGGGSGGGGSGGGGS

(19) United States
(12) Patent Application Publication
(10) Pub. No.: US 2025/0270317 A1
(43) Pub. Date: Aug. 28, 2025

(54) CANCER THERAPY TARGETING NKG2A

(71) Applicant: LES LABORATOIRES SERVIER, SURESNES (FR)

(72) Inventors: Daniel ANDERSEN, Copenhagen (DK); Bruno LAUGEL, Cour sur Loire (FR); Eva Maria Carlsen MELANDER, Limhamn (SE); Vanessa NANCY-PORTEBOIS, Pavillons-Sous-Bois (FR); Alberto OCANA FERNANDEZ, ALBACETE (ES); Marie-Jeanne PIERRAT, NANTERRE (FR); Franziska Katharina UHLENBROCK, Værløse (DK)

(73) Assignee: LES LABORATOIRES SERVIER, Suresnes Cedex (FR)

(21) Appl. No.: 18/698,183

(22) PCT Filed: Oct. 3, 2022
(86) PCT No.: PCT/EP2022/077455

§ 371 Date: Apr. 3, 2024

(30) Foreign Application Priority Data
Oct. 4, 2021 (EP) 21306386.0

(51) Int. Cl. C07K 16/28; A61K 39/00; A61K 45/06; A61P 35/00; A61P 37/04; C07K 16/32

(52) U.S. Cl. CPC C07K 16/2803; A61K 45/06; A61P 35/00; A61P 37/04; C07K 16/2818; C07K 16/2827; C07K 16/2863; C07K 16/32; A61K 2039/507; A61K 2039/545

(57) ABSTRACT

This invention relates to anti-NKG2A antibodies, optionally in combination with anti-PD-1 or anti-PD-L1 antibodies and/or anti-EGFR or anti-HER2 antibodies, and methods of using the antibodies or antibody combinations in enhancing immunity in a patient in need thereof and in treating cancer.

### CROSS-REFERENCE TO RELATED APPLICATIONS

This application is a national stage application under 35 U.S.C. § 371 of International Patent Application No. PCT/EP2022/077455, filed Oct. 3, 2022, which claims the benefit of European Patent Application No. 21306386.0, filed Oct. 4, 2021. The disclosures of those applications are incorporated by reference herein in their entirety.

### SEQUENCE LISTING

The instant application contains a Sequence Listing which has been submitted electronically in XML format and is hereby incorporated by reference in its entirety. Said XML copy, created on Nov. 17, 2022, is named 027628_US004_SL.xml and is 228,408 bytes in size.

### BACKGROUND OF THE INVENTION

Cancer ranks as a leading cause of death and an important barrier to increasing life expectancy in every country of the world. According to estimates from the World Health Organization (WHO) in 2019, cancer is the first or second leading cause of death before the age of 70 years in 112 of 183 countries and ranks third or fourth in a further 23 countries.

HER2-overexpressing gastric cancer is an area of unmet medical need with limited treatment options. Amplification of the HER2 (also known as ERBB2) oncogene and overexpression of the HER2 protein occur in approximately 17-20% of patients with gastric cancers. Patients with HER2-overexpressing gastric cancer benefit from treatment with the anti-HER2 antibody trastuzumab in combination with cisplatin and 5FU or capecitabine in first line, however, after progression on trastuzumab-based therapy, options are limited for treating HER2+ advanced gastric cancer.

Treatment for metastatic colorectal cancer (mCRC) is heavily dependent on gene expression, with many drugs being prescribed only for specific tumor profiles. Anti-PD-1 agents such as nivolumab and pembrolizumab have seen initial success in microsatellite instability high/defective mismatch repair (MSI-H/dMMR) mCRCs, although these comprise only 15% of patients. Tumors that are RAS/BRAF wild-type (WT) and EGFR-positive are among the most common gene expression profiles, accounting for approximately 40% of CRCs.

NKG2A (CD159a) is a C-type lectin that heterodimerizes with CD94, creating an immune inhibitory receptor expressed on natural killer (NK) cells, NKT cells, gamma-delta (γδ) T cells and a subset of cytotoxic T cells (Borrego et al., Immunol Res (2006) 35(3):263-78; Vivier et al., Nat Rev Immunol. (2004) 4(3):190-8). Upon ligation to its ligand, human leucocyte antigen (HLA)-E, NKG2A/CD94 transmits an inhibitory signal via the two immune tyrosine-based inhibition motifs in its cytoplasmic tail and recruitment of SHP-1 tyrosine phosphatase (Carotta et al., Front Immunol. (2016) 7:152). This mechanism is part of natural self-recognition/tolerance by NK cells. However, cancer cells take advantage of this system by overexpressing HLA-E, thereby protecting themselves against NK and T-cell mediated killing. In patients, NKG2A expression is increased on tumor infiltrating NK and T cells and can be induced by immunosuppressive factors such as TGF-β and adenosine (Platonova et al., Cancer Res. (2011) 71(16):5412-22; Sheu et al., Cancer Res. (2005) 65(7):2921-9). Indeed, high intratumoral expression of NKG2A and HLA-E predicts a poor prognosis for patients with liver cancer (HCC) (Sun et al., Oncoimmunology (2017) 6(1):e1264562).

In view of the critical role of NKG2A, PD-1 and PD-L1, HER2, and EGFR in cancer, there is a need for new and improved therapies that target these receptors (e.g., in combination) to treat cancer.

### SUMMARY OF THE INVENTION

The present invention is based on therapies for enhancing immunity comprising an anti-NKG2A antibody, e.g., as described herein, optionally with an antibody targeting PD-1 or PD-L1 and/or an antibody targeting EGFR or HER2, e.g., as described herein. In some embodiments, the therapy is for treating cancer. Also provided are pharmaceutical compositions comprising the components of the therapies, and use of the therapies for enhancing immunity (e.g., treating cancer) in a patient. The therapies described herein may be used in a method for enhancing immunity (e.g., treating cancer) in a patient; may be used for the manufacture of a medicament for enhancing immunity (e.g., treating cancer) in a patient; or may be for use in enhancing immunity (e.g., treating cancer) in a patient. Compared to currently available treatments (e.g., for cancer), including antibody treatments, it is contemplated that the therapies described herein may provide a superior clinical response.

In some embodiments, the present disclosure provides a method of enhancing immunity in a human patient in need thereof, comprising administering to the patient
a) an anti-NKG2A antibody or an antigen-binding portion thereof that competes or cross-competes for binding to human NKG2A with, or binds to the same epitope of human NKG2A as, an antibody that comprises the heavy and light chain amino acid sequences of SEQ ID NOs: 9 and 10, respectively, or SEQ ID NOs: 19 and 20, respectively; and optionally
b) an anti-PD-1 antibody or an anti-PD-L1 antibody, or an antigen-binding portion thereof; and optionally
c) an anti-EGFR antibody component (“anti-EGFR component”) comprising one or two anti-EGFR antibodies or antigen-binding portions thereof, or an anti-HER2 antibody.

In certain embodiments, the method comprises administering:
a) an anti-NKG2A antibody or an antigen-binding portion thereof and an anti-PD-1 antibody or an antigen-binding portion thereof;
b) an anti-NKG2A antibody or an antigen-binding portion thereof and an anti-PD-L1 antibody or an antigen-binding portion thereof;
c) an anti-NKG2A antibody or an antigen-binding portion thereof, an anti-PD-1 antibody or an antigen-binding portion thereof, and an anti-EGFR component;
d) an anti-NKG2A antibody or an antigen-binding portion thereof, an anti-PD-1 antibody or an antigen-binding portion thereof, and an anti-HER2 antibody or an antigen-binding portion thereof;
e) an anti-NKG2A antibody or an antigen-binding portion thereof, an anti-PD-L1 antibody or an antigen-binding portion thereof, and an anti-EGFR component; or
f) an anti-NKG2A antibody or an antigen-binding portion thereof, an anti-PD-L1 antibody or an antigen-binding portion thereof, and an anti-HER2 antibody or an antigen-binding portion thereof.

In some embodiments, the heavy chain complementarity-determining regions (H-CDR) 1-3 and light chain complementarity-determining regions (L-CDR) 1-3 of the anti-NKG2A antibody comprise the amino acid sequences of SEQ ID NOs: 1-6, respectively; or SEQ ID NOs: 11-16, respectively. In certain embodiments, the heavy chain variable domain (VH) and light chain variable domain (VL) of the anti-NKG2A antibody comprise the amino acid sequences of SEQ ID NOs: 7 and 8, respectively; or SEQ ID NOs: 17 and 18, respectively. In particular embodiments, the heavy chain (HC) and light chain (LC) of the anti-NKG2A antibody comprise the amino acid sequences of SEQ ID NOs: 9 and 10, respectively; or SEQ ID NOs: 19 and 20, respectively.

In some embodiments, the H-CDR1-3 and L-CDR1-3 of the anti-PD-1 antibody comprise the amino acid sequences of:
a) SEQ ID NOs: 21-26, respectively;
b) SEQ ID NOs: 31-36, respectively;
c) SEQ ID NOs: 41-46, respectively;
d) SEQ ID NOs: 51-56, respectively;
e) SEQ ID NOs: 61-66, respectively; or
f) SEQ ID NOs: 71-76, respectively.

In certain embodiments, the VH and VL of the anti-PD-1 antibody comprise the amino acid sequences of:
a) SEQ ID NOs: 27 and 28, respectively;
b) SEQ ID NOs: 37 and 38, respectively;
c) SEQ ID NOs: 47 and 48, respectively;
d) SEQ ID NOs: 57 and 58, respectively;
e) SEQ ID NOs: 67 and 68, respectively; or
f) SEQ ID NOs: 77 and 78, respectively.

In particular embodiments, the HC and LC of the anti-PD-1 antibody comprise the amino acid sequences of:
a) SEQ ID NOs: 29 and 30, respectively;
b) SEQ ID NOs: 39 and 40, respectively;
c) SEQ ID NOs: 49 and 50, respectively;
d) SEQ ID NOs: 59 and 60, respectively;
e) SEQ ID NOs: 69 and 70, respectively; or
f) SEQ ID NOs: 79 and 80, respectively.

The anti-PD-1 antibody may be, for example, nivolumab, pembrolizumab, cemiplimab, dostarlimab, or retifanlimab.

In some embodiments, H-CDR1-3 and L-CDR1-3 of the anti-PD-L1 antibody comprise the amino acid sequences of:
a) SEQ ID NOs: 81-86, respectively;
b) SEQ ID NOs: 91-96, respectively; or
c) SEQ ID NOs: 101-106, respectively.

In certain embodiments, the VH and VL of the anti-PD-L1 antibody comprise the amino acid sequences of:
a) SEQ ID NOs: 87 and 88, respectively;
b) SEQ ID NOs: 97 and 98, respectively; or
c) SEQ ID NOs: 107 and 108, respectively.

In particular embodiments, the HC and LC of the anti-PD-L1 antibody comprise the amino acid sequences of:
a) SEQ ID NOs: 89 and 90, respectively;
b) SEQ ID NOs: 99 and 100, respectively; or
c) SEQ ID NOs: 109 and 110, respectively.

The anti-PD-L1 antibody may be, for example, atezolizumab, avelumab, or durvalumab.

In some embodiments, the anti-EGFR component comprises an anti-EGFR antibody or an antigen-binding portion thereof with heavy chain complementarity-determining regions (H-CDR) 1-3 and light chain complementarity-determining regions (L-CDR) 1-3 that comprise the amino acid sequences of:
a) SEQ ID NOs: 111-116, respectively;
b) SEQ ID NOs: 121-126, respectively;
c) SEQ ID NOs: 131-136, respectively; or
d) SEQ ID NOs: 141-146, respectively.

In certain embodiments, the anti-EGFR antibody or antigen-binding portion thereof comprises a VH and VL that comprise the amino acid sequences of:
a) SEQ ID NOs: 117 and 118, respectively;
b) SEQ ID NOs: 127 and 128, respectively;
c) SEQ ID NOs: 137 and 138, respectively; or
d) SEQ ID NOs: 147 and 148, respectively.

In particular embodiments, the anti-EGFR antibody comprises an HC and an LC that comprise the amino acid sequences of:
a) SEQ ID NOs: 119 and 120, respectively;
b) SEQ ID NOs: 129 and 130, respectively;
c) SEQ ID NOs: 139 and 140, respectively; or
d) SEQ ID NOs: 149 and 150, respectively.

The anti-EGFR antibody may be, for example, cetuximab, panitumumab, futuximab, or modotuximab.

In some embodiments, the anti-EGFR component comprises an anti-EGFR antibody or an antigen-binding portion thereof with H-CDR1-3 and L-CDR1-3 that comprise the amino acid sequences of SEQ ID NOs: 131-136, respectively, and an anti-EGFR antibody or an antigen-binding portion thereof with H-CDR1-3 and L-CDR1-3 that comprise the amino acid sequences of SEQ ID NOs: 141-146, respectively. In certain embodiments, the anti-EGFR component comprises an anti-EGFR antibody or an antigen-binding portion thereof with a VH and VL that comprise the amino acid sequences of SEQ ID NOs: 137 and 138, respectively, and an anti-EGFR antibody or an antigen-binding portion thereof with a VH and VL that comprise the amino acid sequences of SEQ ID NOs: 147 and 148, respectively. In particular embodiments, the anti-EGFR component comprises an anti-EGFR antibody with a heavy chain (HC) and light chain (LC) that comprise the amino acid sequences of SEQ ID NOs: 139 and 140, respectively, and an anti-EGFR antibody with an HC and LC that comprise the amino acid sequences of SEQ ID NOs: 149 and 150, respectively. The anti-EGFR component may be, for example, futuximab+modotuximab (e.g., in a 1:1 ratio).

In some embodiments, the H-CDR1-3 and the L-CDR1-3 of the anti-HER2 antibody comprise the amino acid sequences of SEQ ID NOs: 151-156, respectively; or SEQ ID NOs: 161-166, respectively. In certain embodiments, the VH and VL of the anti-HER2 antibody comprise the amino acid sequences of SEQ ID NOs: 157 and 158, respectively; or SEQ ID NOs: 167 and 168, respectively. In particular embodiments, the HC and LC of the anti-HER2 antibody comprise the amino acid sequences of SEQ ID NOs: 159 and 160, respectively; or SEQ ID NOs: 169 and 170, respectively. In some embodiments, the anti-HER2 antibody or antigen-binding portion may be conjugated to a moiety such as DXd or DM1. The anti-HER2 antibody may be, for example, trastuzumab, margetuximab, trastuzumab dexrutecan, or trastuzumab emtansine.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; and an anti-PD-1 antibody or an antigen-binding portion thereof comprising the H-CDR1-3 and L-CDR1-3 amino acid sequences of SEQ ID NOs: 61-66, respectively;
b) an anti-NKG2A antibody or an antigen-binding portion thereof comprising the VH and VL amino acid sequences of SEQ ID NOs: 7 and 8, respectively; and an anti-PD-1 antibody or an antigen-binding portion thereof comprising the VH and VL amino acid sequences of SEQ ID NOs: 67 and 68, respectively; or
c) an anti-NKG2A antibody comprising the HC and LC amino acid sequences of SEQ ID NOs: 9 and 10, respectively; and an anti-PD-1 antibody comprising the HC and LC amino acid sequences of SEQ ID NOs: 69 and 70, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively;
   an anti-PD-1 antibody or an antigen-binding portion thereof comprising the H-CDR1-3 and L-CDR1-3 amino acid sequences of SEQ ID NOs: 61-66, respectively; and
   an anti-EGFR component comprising an anti-EGFR antibody or an antigen-binding portion thereof comprising the H-CDR1-3 and L-CDR1-3 amino acid sequences of SEQ ID NOs: 131-136, respectively, and an anti-EGFR antibody or an antigen-binding portion thereof comprising the H-CDR1-3 and L-CDR1-3 amino acid sequences of SEQ ID NOs: 141-146, respectively;
b) an anti-NKG2A antibody or an antigen-binding portion thereof comprising the VH and VL amino acid sequences of SEQ ID NOs: 7 and 8, respectively;
   an anti-PD-1 antibody or an antigen-binding portion thereof comprising the VH and VL amino acid sequences of SEQ ID NOs: 67 and 68, respectively; and
   an anti-EGFR component comprising an anti-EGFR antibody or an antigen-binding portion thereof comprising the VH and VL amino acid sequences of SEQ ID NOs: 137 and 138, respectively, and an anti-EGFR antibody or an antigen-binding portion thereof comprising the VH and VL amino acid sequences of SEQ ID NOs: 147 and 148, respectively; or
c) an anti-NKG2A antibody comprising the HC and LC amino acid
   sequences of SEQ ID NOs: 9 and 10, respectively;
an anti-PD-1 antibody comprising the HC and LC amino acid sequences of SEQ ID NOs: 69 and 70, respectively; and
   an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 61-66, respectively; and an anti-HER2 antibody or an antigen-binding portion thereof comprising the H-CDR1-3 and L-CDR1-3 amino acid sequences of SEQ ID NOs: 161-166, respectively;
b) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 67 and 68, respectively; and an anti-HER2 antibody or an antigen-binding portion thereof comprising the VH and VL amino acid sequences of SEQ ID NOs: 167 and 168, respectively; or
c) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 69 and 70, respectively; and an anti-HER2 antibody comprising the HC and LC amino acid sequences of SEQ ID NOs: 169 and 170, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 71-76, respectively; and an anti-HER2 antibody or an antigen-binding portion thereof comprising the H-CDR1-3 and L-CDR1-3 amino acid sequences of SEQ ID NOs: 161-166, respectively;
b) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 77 and 78, respectively; and an anti-HER2 antibody or an antigen-binding portion thereof comprising the VH and VL amino acid sequences of SEQ ID NOs: 167 and 168, respectively; or
c) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 79 and 80, respectively; and an anti-HER2 antibody comprising the HC and LC amino acid sequences of SEQ ID NOs: 169 and 170, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-HER2 antibody or an antigen-binding portion thereof comprising the H-CDR1-3 and L-CDR1-3 amino acid sequences of SEQ ID NOs: 151-156, respectively;
b) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-HER2 antibody or an antigen-binding portion thereof comprising the VH and VL amino acid sequences of SEQ ID NOs: 157 and 158, respectively; or
c) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-HER2 antibody comprising the HC and LC amino acid sequences of SEQ ID NOs: 159 and 160, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 21-26, respectively; and an anti-HER2 antibody or an antigen-binding portion thereof comprising the H-CDR1-3 and L-CDR1-3 amino acid sequences of SEQ ID NOs: 151-156, respectively;
b) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 27 and 28, respectively; and an anti-HER2 antibody or an antigen-binding portion thereof comprising the VH and VL amino acid sequences of SEQ ID NOs: 157 and 158, respectively; or
c) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 29 and 30, respectively; and an anti-HER2 antibody comprising the HC and LC amino acid sequences of SEQ ID NOs: 159 and 160, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively;
   an anti-PD-1 antibody or an antigen-binding portion thereof comprising the H-CDR1-3 and L-CDR1-3 amino acid sequences of SEQ ID NOs: 31-36, respectively; and
   an anti-EGFR component comprising an anti-EGFR antibody or an antigen-binding portion thereof comprising the H-CDR1-3 and L-CDR1-3 amino acid sequences of SEQ ID NOs: 131-136, respectively, and an anti-EGFR antibody or an antigen-binding portion thereof comprising the H-CDR1-3 and L-CDR1-3 amino acid sequences of SEQ ID NOs: 141-146, respectively;
b) an anti-NKG2A antibody or an antigen-binding portion thereof comprising the VH and VL amino acid sequences of SEQ ID NOs: 7 and 8, respectively;
   an anti-PD-1 antibody or an antigen-binding portion thereof comprising the VH and VL amino acid sequences of SEQ ID NOs: 37 and 38, respectively; and
   an anti-EGFR component comprising an anti-EGFR antibody or an antigen-binding portion thereof comprising the VH and VL amino acid sequences of SEQ ID NOs: 137 and 138, respectively, and an anti-EGFR antibody or an antigen-binding portion thereof comprising the VH and VL amino acid sequences of SEQ ID NOs: 147 and 148, respectively; or
c) an anti-NKG2A antibody comprising the HC and LC amino acid sequences of SEQ ID NOs: 9 and 10, respectively;
   an anti-PD-1 antibody comprising the HC and LC amino acid sequences of SEQ ID NOs: 39 and 40, respectively; and
   an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively;
   an anti-PD-1 antibody or an antigen-binding portion thereof comprising the H-CDR1-3 and L-CDR1-3 amino acid sequences of SEQ ID NOs: 21-26, respectively; and
   an anti-EGFR component comprising an anti-EGFR antibody or an antigen-binding portion thereof comprising the H-CDR1-3 and L-CDR1-3 amino acid sequences of SEQ ID NOs: 131-136, respectively, and an anti-EGFR antibody or an antigen-binding portion thereof comprising the H-CDR1-3 and L-CDR1-3 amino acid sequences of SEQ ID NOs: 141-146, respectively;
b) an anti-NKG2A antibody or an antigen-binding portion thereof comprising the VH and VL amino acid sequences of SEQ ID NOs: 7 and 8, respectively;
   an anti-PD-1 antibody or an antigen-binding portion thereof comprising the VH and VL amino acid sequences of SEQ ID NOs: 27 and 28, respectively; and
   an anti-EGFR component comprising an anti-EGFR antibody or an antigen-binding portion thereof comprising the VH and VL amino acid sequences of SEQ ID NOs: 137 and 138, respectively, and an anti-EGFR antibody or an antigen-binding portion thereof comprising the VH and VL amino acid sequences of SEQ ID NOs: 147 and 148, respectively; or
c) an anti-NKG2A antibody comprising the HC and LC amino acid sequences of SEQ ID NOs: 9 and 10, respectively;
   an anti-PD-1 antibody comprising the HC and LC amino acid sequences of SEQ ID NOs: 29 and 30, respectively; and an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-EGFR antibody or an antigen-binding portion thereof comprising the H-CDR1-3 and L-CDR1-3 amino acid sequences of SEQ ID NOs: 111-116, respectively;
b) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-EGFR antibody or an antigen-binding portion thereof comprising the VH and VL amino acid sequences of SEQ ID NOs: 117 and 118, respectively; or
c) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-EGFR antibody comprising the HC and LC amino acid sequences of SEQ ID NOs: 119 and 120, respectively.

In some embodiments, the method comprises administering to the patient:
a) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 21-26, respectively; and an anti-EGFR antibody or an antigen-binding portion thereof comprising the H-CDR1-3 and L-CDR1-3 amino acid sequences of SEQ ID NOs: 111-116, respectively;
b) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 27 and 28, respectively; and an anti-EGFR antibody or an antigen-binding portion thereof comprising the VH and VL amino acid sequences of SEQ ID NOs: 117 and 118, respectively; or
c) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 29 and 30, respectively; and an anti-EGFR antibody comprising the HC and LC amino acid sequences of SEQ ID NOs: 119 and 120, respectively.

In any of the above methods, the antibodies or antigen-binding portions may be administered to the patient concurrently or sequentially.

In some embodiments, the patient has cancer, e.g., a hematological malignancy or a solid tumor. In certain embodiments, the cancer is colorectal cancer or gastric cancer.

In some embodiments, the anti-NKG2A antibody or antigen-binding portion thereof is administered at a dose of 8, 20, 100, 300, 750, or 1500 mg (e.g., every two weeks). The antibody or antigen-binding portion may be administered in a 28-day cycle. In some embodiments, the anti-PD-1 or anti-PD-L1 antibody, or antigen-binding portion thereof, is administered at a dose of 200 mg (e.g., every two weeks), and in some cases may be administered after one cycle of the anti-NKG2A antibody or antigen-binding portion. In some embodiments, the anti-EGFR component is administered at a dose of 6 mg/kg, 9 mg/kg, or a loading dose of 9 mg/kg followed by 6 mg/kg (e.g., weekly or every two weeks). In some embodiments, the anti-HER2 antibody or antigen-binding portion thereof is administered at a dose of 15 mg/kg (e.g., every three weeks or every four weeks). In certain embodiments, the antibodies or antigen-binding portions are formulated for intravenous administration (e.g., intravenous infusion).

The present disclosure provides a method of treating an advanced solid tumor malignancy in a patient, comprising administering to the patient an anti-NKG2A antibody comprising the HC and LC amino acid sequences of SEQ ID NOs: 9 and 10, respectively, e.g., wherein the antibody is administered at a dose of 8, 20, 100, 300, 750, or 1500 mg every two weeks by IV infusion.

Further, the present disclosure provides a method of treating an advanced solid tumor malignancy in a patient, comprising administering to the patient:
a) an anti-NKG2A antibody comprising the HC and LC amino acid sequences of SEQ ID NOs: 9 and 10, respectively; and
b) an anti-PD-1 antibody comprising the HC and LC amino acid sequences of SEQ ID NOs: 69 and 70, respectively,
   e.g., wherein the anti-NKG2A antibody is administered at a dose of 20, 100, 300, 750, or 1500 mg every two weeks by IV infusion, and wherein after completing a 28-day cycle of anti-NKG2A antibody administration, the anti-PD-1 antibody is administered at a dose of 200 mg every two weeks by IV infusion.

Further, the present disclosure provides a method of treating an advanced solid tumor malignancy in a patient, comprising administering to the patient:
a) an anti-NKG2A antibody comprising the HC and LC amino acid sequences of SEQ ID NOs: 9 and 10, respectively; and
b) an anti-PD-1 antibody comprising the HC and LC amino acid sequences of SEQ ID NOs: 39 and 40, respectively, e.g., wherein the anti-NKG2A antibody is administered at a dose of 20, 100, 300, 750, or 1500 mg every two weeks by IV infusion, and wherein after completing a 28-day cycle of anti-NKG2A antibody administration, the anti-PD-1 antibody is administered at a dose of 200 mg every two weeks by IV infusion.

Further, the present disclosure provides a method of treating an advanced solid tumor malignancy in a patient, comprising administering to the patient:
a) an anti-NKG2A antibody comprising the HC and LC amino acid sequences of SEQ ID NOs: 9 and 10, respectively; and
b) an anti-PD-1 antibody comprising the HC and LC amino acid sequences of SEQ ID NOs: 29 and 30, respectively,
   e.g., wherein the anti-NKG2A antibody is administered at a dose of 20, 100, 300, 750, or 1500 mg every two weeks by IV infusion, and wherein after completing a 28-day cycle of anti-NKG2A antibody administration, the anti-PD-1 antibody is administered at a dose of 200 mg every two weeks by IV infusion.

The present disclosure also provides a method of treating metastatic HER2⁺ gastric cancer in a patient, comprising administering to the patient:
a) an anti-NKG2A antibody comprising the HC and LC amino acid sequences of SEQ ID NOs: 9 and 10, respectively;
b) an anti-PD-1 antibody comprising the HC and LC amino acid sequences of SEQ ID NOs: 69 and 70, respectively; and
c) an anti-HER2 antibody comprising the HC and LC amino acid sequences of SEQ ID NOs: 169 and 170, respectively.
   In some embodiments, the cancer is locally advanced and/or unresectable. In some embodiments, the patient has failed on first-line standard of care therapy. In certain embodiments, the anti-NKG2A antibody is administered at a dose of 20, 100, 300, 750, or 1500 mg every two weeks; after completing a 28-day cycle of anti-NKG2A antibody administration the anti-PD-1 antibody is administered at a dose of 200 mg every two weeks; and the anti-HER2 antibody is administered at 15 mg/kg every three or four weeks, wherein the antibodies are administered via IV infusion.

The present disclosure also provides a method of treating metastatic HER2+ gastric cancer in a patient, comprising administering to the patient:
a) an anti-NKG2A antibody comprising the HC and LC amino acid sequences of SEQ ID NOs: 9 and 10, respectively;
b) an anti-PD-1 antibody comprising the HC and LC amino acid sequences of SEQ ID NOs: 79 and 80, respectively; and
c) an anti-HER2 antibody comprising the HC and LC amino acid sequences of SEQ ID NOs: 169 and 170, respectively.
   In some embodiments, the cancer is locally advanced and/or unresectable. In some embodiments, the patient has failed on first-line standard of care therapy. In certain embodiments, the anti-NKG2A antibody is administered at a dose of 20, 100, 300, 750, or 1500 mg every two weeks; after completing a 28-day cycle of anti-NKG2A antibody administration the anti-PD-1 antibody is administered at a dose of 200 mg every two weeks; and the anti-HER2 antibody is administered at 15 mg/kg every three or four weeks, wherein the antibodies are administered via IV infusion.

The present disclosure also provides a method of treating metastatic colorectal cancer in a patient, comprising administering to the patient:
a) an anti-NKG2A antibody comprising the HC and LC amino acid sequences of SEQ ID NOs: 9 and 10, respectively;
b) an anti-PD-1 antibody comprising the HC and LC amino acid sequences of SEQ ID NOs: 69 and 70, respectively; and
c) an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.
   In some embodiments, the patient has low microsatellite instability status. In some embodiments, the patient is (i) without RAS mutations in any of the following codons: codons 12 and 13 in exon 2, codons 59 and 61 in exon 3, and codons 117 and 146 in exon 4; and/or (ii) without the BRAF V600E mutation. In certain embodiments, the anti-NKG2A antibody is administered at a dose of 20, 100, 300, 750, or 1500 mg every two weeks; after completing a 28-day cycle of anti-NKG2A antibody administration, the anti-PD-1 antibody is administered at a dose of 200 mg every two weeks; and the anti-EGFR component is administered at a loading dose of 9 mg/kg followed by a dose of 6 mg/kg every one or two weeks, wherein the antibodies are administered via IV infusion.

The present disclosure also provides a method of treating metastatic colorectal cancer in a patient, comprising administering to the patient:
a) an anti-NKG2A antibody comprising the HC and LC amino acid sequences of SEQ ID NOs: 9 and 10, respectively;
b) an anti-PD-1 antibody comprising the HC and LC amino acid sequences of SEQ ID NOs: 39 and 40, respectively; and
c) an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.
   In some embodiments, the patient has low microsatellite instability status. In some embodiments, the patient is (i) without RAS mutations in any of the following codons: codons 12 and 13 in exon 2, codons 59 and 61 in exon 3, and codons 117 and 146 in exon 4; and/or (ii) without the BRAF V600E mutation. In certain embodiments, the anti-NKG2A antibody is administered at a dose of 20, 100, 300, 750, or 1500 mg every two weeks; after completing a 28-day cycle of anti-NKG2A antibody administration, the anti-PD-1 antibody is administered at a dose of 200 mg every two weeks; and the anti-EGFR component is administered at a loading dose of 9 mg/kg followed by a dose of 6 mg/kg every one or two weeks, wherein the antibodies are administered via IV infusion.

The present disclosure also provides a method of treating metastatic colorectal cancer in a patient, comprising administering to the patient:
a) an anti-NKG2A antibody comprising the HC and LC amino acid sequences of SEQ ID NOs: 9 and 10, respectively;
b) an anti-PD-1 antibody comprising the HC and LC amino acid sequences of SEQ ID NOs: 29 and 30, respectively; and
c) an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.
   In some embodiments, the patient has low microsatellite instability status. In some embodiments, the patient is (i) without RAS mutations in any of the following codons: codons 12 and 13 in exon 2, codons 59 and 61 in exon 3, and codons 117 and 146 in exon 4; and/or (ii) without the BRAF V600E mutation. In certain embodiments, the anti-NKG2A antibody is administered at a dose of 20, 100, 300, 750, or 1500 mg every two weeks; after completing a 28-day cycle of anti-NKG2A antibody administration, the anti-PD-1 antibody is administered at a dose of 200 mg every two weeks; and the anti-EGFR component is administered at a loading dose of 9 mg/kg followed by a dose of 6 mg/kg every one or two weeks, wherein the antibodies are administered via IV infusion.

In some embodiments, a method of the present disclosure further comprises administering to the patient radiation therapy, or at least one of a chemotherapeutic agent, an anti-neoplastic agent, and an anti-angiogenic agent.

Treatment according to a method of the present disclosure may result in tumor regression, delay of tumor progression, inhibition of cancer progression, inhibition of cancer metastasis, prevention of cancer recurrence or residual disease, and/or prolonged survival. In some embodiments, treatment according to a method of the present disclosure may result in an improved objective response rate, improved clinical benefit rate, improved duration of response, increased progression-free survival, increased overall survival, or any combination thereof, e.g., in comparison to an untreated patient.

The present disclosure also provides a multi-specific antibody that specifically binds to:
a) human NKG2A and human PD-1;
b) human NKG2A and human PD-L1;
c) human NKG2A, human PD-1, and human EGFR;
d) human NKG2A, human PD-1, and human HER2;
e) human NKG2A, human PD-L1, and human EGFR; or
f) human NKG2A, human PD-L1, and human HER2.
   In some embodiments, the multi-specific antibody comprises:
a) an antigen-binding domain of an anti-NKG2A antibody as described herein and an antigen-binding domain of an anti-PD-1 antibody as described herein;
b) an antigen-binding domain of an anti-NKG2A antibody as described herein and an antigen-binding domain of an anti-PD-L1 antibody as described herein;
c) an antigen-binding domain of an anti-NKG2A antibody as described herein, an antigen-binding domain of an anti-PD-1 antibody as described herein, and an antigen-binding portion of one or two anti-EGFR antibodies as described herein;
d) an antigen-binding domain of an anti-NKG2A antibody as described herein, an antigen-binding domain of an anti-PD-1 antibody as described herein, and an antigen-binding portion of an anti-HER2 antibody as described herein;
e) an antigen-binding domain of an anti-NKG2A antibody as described herein, an antigen-binding domain of an anti-PD-L1 antibody as described herein, and an antigen-binding portion of one or two anti-EGFR antibodies as described herein; or
f) an antigen-binding domain of an anti-NKG2A antibody as described herein, an antigen-binding domain of an anti-PD-L1 antibody as described herein, and an antigen-binding portion of an anti-HER2 antibody as described herein.

The present disclosure also provides a pharmaceutical composition comprising an anti-NKG2A antibody or an antigen-binding portion thereof as described herein, and further comprising:
a) an anti-PD-1 antibody or an antigen-binding portion thereof;
b) an anti-PD-L1 antibody or an antigen-binding portion thereof;
c) an anti-PD-1 antibody or an antigen-binding portion thereof and an anti-EGFR component;
d) an anti-PD-1 antibody or an antigen-binding portion thereof and an anti-HER2 antibody or an antigen-binding portion thereof;
e) an anti-PD-L1 antibody or an antigen-binding portion thereof and an anti-EGFR component; or
f) an anti-PD-L1 antibody or an antigen-binding portion thereof and an anti-HER2 antibody or an antigen-binding portion thereof;
   and a pharmaceutically acceptable excipient. The anti-PD-1 antibody or antigen-binding portion thereof, anti-PD-L1 antibody or antigen-binding portion thereof, anti-EGFR component, and anti-HER2 antibody or antigen-binding portion thereof may be, e.g., as described herein. In particular embodiments, the pharmaceutical composition may comprise the antibodies or antigen-binding portions of any of the methods described herein, and may be for use in treating a human patient in any of the methods described herein.

The present disclosure also provides an anti-NKG2A antibody or an antigen-binding portion thereof as described herein for use in enhancing immunity in a human patient in need thereof in combination with:
a) an anti-PD-1 antibody or an antigen-binding portion thereof;
b) an anti-PD-L1 antibody or an antigen-binding portion thereof;
c) an anti-PD-1 antibody or an antigen-binding portion thereof and an anti-EGFR component;
d) an anti-PD-1 antibody or an antigen-binding portion thereof and an anti-HER2 antibody or an antigen-binding portion thereof;
e) an anti-PD-L1 antibody or an antigen-binding portion thereof and an anti-EGFR component; or
f) an anti-PD-L1 antibody or an antigen-binding portion thereof and an anti-HER2 antibody or an antigen-binding portion thereof.
   The anti-PD-1 antibody or antigen-binding portion thereof, anti-PD-L1 antibody or antigen-binding portion thereof, anti-EGFR component, and anti-HER2 antibody or antigen-binding portion thereof may be, e.g., as described herein. In some embodiments, the anti-NKG2A antibody or antigen-binding portion thereof is for use in treating a human patient in a method as described herein.

The present disclosure also provides use of an anti-NKG2A antibody or an antigen-binding portion thereof as described herein in combination with:
a) an anti-PD-1 antibody or an antigen-binding portion thereof;
b) an anti-PD-L1 antibody or an antigen-binding portion thereof;
c) an anti-PD-1 antibody or an antigen-binding portion thereof and an anti-EGFR component;
d) an anti-PD-1 antibody or an antigen-binding portion thereof and an anti-HER2 antibody or an antigen-binding portion thereof;
e) an anti-PD-L1 antibody or an antigen-binding portion thereof and an anti-EGFR component; or
f) an anti-PD-L1 antibody or an antigen-binding portion thereof and an anti-HER2 antibody or an antigen-binding portion thereof.
   for the manufacture of a medicament for enhancing immunity in a human patient in need thereof. The anti-PD-1 antibody or antigen-binding portion thereof, anti-PD-L1 antibody or antigen-binding portion thereof, anti-EGFR component, and anti-HER2 antibody or antigen-binding portion thereof may be, e.g., as described herein. In some embodiments, the medicament is for treating a human patient in a method as described herein.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a pair of graphs showing the expression of endogenous HLA-E at the surface of six different tumor cell lines (Panel A) and the effect of mAb1 on NK-mediated killing of these six tumor cell lines (Panel B).

FIG. 2 is a pair of graphs showing γδ T-cell mediated tumor cell killing by mAb1 compared to a monalizumab analogue (Panel A) and analogues of BMS anti-NKG2A mAbs (Panel B) in vitro. One representative donor depicted, results were verified using n=3 donors. Data are presented as means±SEM.

FIG. 3 is a set of graphs showing the percentage specific lysis of FaDu cells (HLA-E+/EGFR+) by cetuximab titrated in a dose-dependent manner alone or in combination with mAb1, monalizumab analogue, or isotype controls.

FIG. 4A is a pair of graphs showing the mAb1-induced potentiation of NK cell-mediated killing of A431 tumor cells in combination with the anti-EGFR antibody cetuximab in two human donors (D1 and D2). Data are presented as means±SEM.

FIG. 4B is a pair of graphs showing the mAb1-induced potentiation of NK cell-mediated killing of A431 tumor cells in combination with the anti-EGFR antibody combination futuximab/modotuximab (“Futux/Modo”) in two human donors (D1 and D2). Data are presented as means±SEM.

FIG. 5 is a pair of graphs showing NK cell activation (as assessed by CD137 expression) effected by mAb1 alone or in combination with the anti-EGFR antibody cetuximab (“cetux”) (Panel A) or the anti-EGFR antibody combination futuximab/modotuximab (“futux/modo”) (Panel B). Each data point represents a donor.

FIG. 6 is a graph showing secretion of IFNy by primary NK cells in vitro in the presence of A431 cells, IL-2, and the antibodies or antibody combinations shown. Each data point represents a donor.

FIG. 7 is a pair of graphs showing the mAb1-induced potentiation of NK cell-mediated killing of A431 or MDA-MB-231 tumor cells in combination with the anti-PD-L1 antibody avelumab in two human donors (D1 and D2). Data are presented as means±SEM.

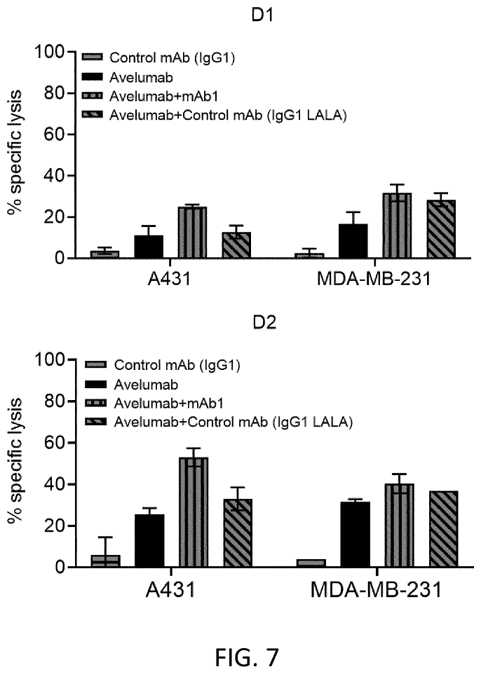

FIG. 8 is a pair of graphs showing the effects of mAb1 in combination with avelumab (“Ave”) on induction of NK cell activation (CD137) (Panel A) and IFNγ secretion (Panel B).

FIG. 9A is a graph showing tumor growth in CD34 humanized mice subcutaneously engrafted with MDA-MB-231 human breast tumor cells. The grey area indicates the treatment period. Data are presented as means±SEM. *p<0.05.

FIG. 9B is a set of graphs showing flow cytometric analysis of MDA-MB-231 tumor infiltrating lymphocytes. The percentage of human CD45+ and CD3+ cells and the ratio of CD8/CD4 T cells is shown for tumors treated with the antibody or antibody combinations shown as compared to vehicle. Numbers are presented as percentage of live cells (CD45+), human CD45+ cells (CD3+), or a ratio (CD4 and CD8).

FIG. 10 is a diagram showing the design of a clinical study of mAb1 monotherapy and combination therapy.

FIG. 11 shows percent of tumor eradication in hNKG2A/hCD94 KI mice subcutaneously engrafted with MC38-HLAE murine tumor cells. The mice were treated three times weekly with a total of nine doses. Tumor volume was measured three times weekly. The grey area denotes the treatment period. Data are presented as means±SEM.

FIG. 12 shows trastuzumab and mAb1 induced NK cell secretion of MIP-13. Human primary NKG2A+NK cells from healthy individuals were cocultured with HLA-E transduced N87, BxPC3, SKOV3, A375, A549 and JIMT-1 target cells for 48 hours in the presence of 10 ng/mL IL-2. The secretion of MIP-1p in coculture supernatants was quantified by ELISA.

FIG. 13 is a set of graphs showing the combinatorial effect of double PD-1 and NKG2A blockade on peripheral blood lymphocytes (PBLs) isolated from 3 healthy donors (A, B and C). PBLs were co-cultured with the HER2 positive SKOV3 cells overexpressing HLA-E as well as PD-L1 and activated with trastuzumab or trastuzumab plus zoledronate.

FIG. 14 is a diagram showing the design of a clinical study of mAb1+ pembrolizumab in the treatment of colorectal cancer.

FIG. 15 is a diagram showing the design of a clinical study of mAb1+ pembrolizumab+trastuzumab in the treatment of gastric cancer.

### DETAILED DESCRIPTION OF THE INVENTION

The present disclosure provides new monotherapies and compositions that target human NKG2A, and new combination therapies and compositions that target human NKG2A; human PD-1 or PD-L1; and/or human EGFR or HER2, by using antibodies that bind these targets. The therapies (i.e., monotherapies and combination therapies) and compositions can be used to treat cancer in a human patient. Unless otherwise stated, as used herein, “NKG2A,” “PD-1,” “PD-L1,” “EGFR,” and “HER2” refer to the human forms of those targets. A human NKG2A polypeptide sequence is available under UniProt Accession No. P26715 (SEQ ID NO: 171). A human PD-1 polypeptide sequence is available under UniProt Accession No. Q15116 (SEQ ID NO: 172). A human PD-L1 polypeptide sequence is available under UniProt Accession No. Q9NZQ7 (SEQ ID NO: 173). A human EGFR polypeptide sequence is available under UniProt Accession No. P00533 (SEQ ID NO: 174). A human HER2 polypeptide sequence is available under UniProt Accession No. P04626 (SEQ ID NO: 175). These sequences are shown in Table 4.

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. The assignment of amino acid numbers, and of FR and CDR regions, in the heavy or light chain may be in accordance with IMGT® definitions (Lefranc et al., Dev Comp Immunol (2003) 27(1):55-77); Eu numbering; or the definitions of Kabat, Sequences of Proteins of Immunological Interest (National Institutes of Health, Bethesda, MD (1987 and 1991)); Chothia & Lesk, J. Mol. Biol. (1987) 196:901-17; Chothia et al., Nature (1989) 342:878-83; MacCallum et al., J. Mol. Biol. (1996) 262:732-45; or Honegger and Plückthun, J. Mol. Biol. (2001) 309(3):657-70.

The term “recombinant antibody” refers to an antibody that is expressed from a cell or cell line comprising the nucleotide sequence(s) that encode the antibody, wherein said nucleotide sequence(s) are not naturally associated with the cell.

The term “isolated protein” “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between an antigen and an antibody. The intrinsic attractiveness of the antibody for the antigen is typically expressed as the binding affinity equilibrium constant (K_{D}) of a particular antibody-antigen interaction. An antibody is said to specifically bind to an antigen when the K_{D} for the binding is ≤1 μM, e.g., ≤100 nM or ≤10 nM. A K_{D} binding affinity constant can be measured, e.g., by surface plasmon resonance (BIAcore™) using the IBIS MX96 SPR system from IBIS Technologies or the Carterra LSA SPR platform, or by Bio-Layer Interferometry, for example using the Octet™ system from ForteBio.

The term “epitope” as used herein refers to a portion (determinant) of an antigen that specifically binds to an antibody or a related molecule such as a bi-specific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest or a relevant portion thereof, then screening for binding to the epitope.

One can determine whether an antibody binds to the same epitope as or competes for binding with an antibody as described herein by using methods known in the art, including, without limitation, competition assays, epitope binning, and alanine scanning. In some embodiments, one allows an antibody of the present disclosure to bind to its target under saturating conditions, and then measures the ability of the test antibody to bind to the target. If the test antibody is able to bind to the target at the same time as the reference antibody, then the test antibody binds to a different epitope than the reference antibody. However, if the test antibody is not able to bind to the target at the same time, then the test antibody binds to the same epitope, an overlapping epitope, or an epitope that is in close proximity to the epitope bound by the antibody of the present disclosure. This experiment can be performed using, e.g., ELISA, RIA, BIACORE™, SPR, Bio-Layer Interferometry or flow cytometry. To test whether an antibody cross-competes with another antibody, one may use the competition method described above in two directions, i.e., determining if the known antibody blocks the test antibody and vice versa. Such cross-competition experiments may be performed, e.g., using an IBIS MX96 or Carterra LSA SPR instrument or the Octet™ system.

The term “antigen-binding portion” of an antibody (or simply “antibody portion”), as used herein, refers to one or more portions or fragments of an antibody that retain the ability to specifically bind to an antigen. It has been shown that certain fragments of a full-length antibody can perform the antigen-binding function of the antibody. Examples of binding fragments encompassed within the term “antigen-binding portion” include (i) a Fab fragment: a monovalent fragment consisting of the VL, VH, CL and CH1 domains; (ii) a F(ab′)2 fragment: a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated complementarity determining region (CDR) capable of specifically binding to an antigen. Furthermore, although the two domains of the Fv fragment, VL and VH, are encoded by separate genes, they can be joined, using recombinant methods, by a synthetic linker that enables them to be made as a single protein chain in which the VL and VH domains pair to form monovalent molecules (known as single chain Fv (scFv)). Also within the present disclosure are antigen-binding molecules comprising a VH and/or a VL. In the case of a VH, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bi-specific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesin molecules can be obtained using standard recombinant DNA techniques, e.g., as described herein.

The class (isotype) and subclass of antibodies may be determined by any method known in the art. In general, the class and subclass of an antibody may be determined using antibodies that are specific for a particular class and subclass of antibody. Such antibodies are available commercially. The class and subclass can be determined by ELISA or Western blot as well as other techniques. Alternatively, the class and subclass may be determined by sequencing all or a portion of the constant regions of the heavy and/or light chains of the antibodies, comparing their amino acid sequences to the known amino acid sequences of various classes and subclasses of immunoglobulins, and determining the class and subclass of the antibodies.

Unless otherwise indicated, all antibody amino acid residue numbers referred to in this disclosure are those under the IMGT® numbering scheme.

### Anti-NKG2A Antibodies

In some embodiments, a therapy (e.g., a monotherapy or a combination therapy) or composition described herein comprises an anti-NKG2A antibody or an antigen-binding portion thereof. In certain embodiments, the anti-NKG2A antibody is the antibody referred to herein as antibody mAb1 or mAb2 or a variant of any of these, where the variant may contain, e.g., certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of the antibody. mAb1 comprises heavy and light chain amino acid sequences of SEQ ID NOs: 9 and 10, respectively. mAb2 comprises heavy and light chain amino acid sequences of SEQ ID NOs: 19 and 20, respectively.

In some embodiments, the anti-NKG2A antibody competes or cross-competes for binding to human NKG2A with, or binds to the same epitope of human NKG2A as, antibody mAb1 or mAb2.

In some embodiments, the anti-NKG2A antibody comprises H-CDR1-3 comprising the amino acid sequences, respectively, of SEQ ID NOs: 1-3 or 11-13.

In some embodiments, the anti-NKG2A antibody has a VH that is at least 90% (e.g., at least 92%, at least 95%, at least 96%, at least 97%, at least 98%, or at least 99%) identical to the amino acid sequence of SEQ ID NO: 7 or 17. In certain embodiments, the anti-NKG2A antibody has a VH that comprises the amino acid sequence of SEQ ID NO: 7 or 17.

In some embodiments, the anti-NKG2A antibody has an HC that is at least 90% (e.g., at least 92%, at least 95%, at least 96%, at least 97%, at least 98%, or at least 99%) identical to the amino acid sequence of SEQ ID NO: 9 or 19. In certain embodiments, the anti-NKG2A antibody has an HC that comprises the amino acid sequence of SEQ ID NO: 9 or 19.

In some embodiments, the anti-NKG2A antibody comprises L-CDR1-3 comprising the amino acid sequences, respectively, of SEQ ID NOs: 4-6 or 14-16.

In some embodiments, the anti-NKG2A antibody has a VL that is at least 90% (e.g., at least 92%, at least 95%, at least 96%, at least 97%, at least 98%, or at least 99%) identical to the amino acid sequence of SEQ ID NO: 8 or 18. In certain embodiments, the anti-NKG2A antibody has a VL that comprises the amino acid sequence of SEQ ID NO: 8 or 18.

In some embodiments, the anti-NKG2A antibody has an LC that is at least 90% (e.g., at least 92%, at least 95%, at least 96%, at least 97%, at least 98%, or at least 99%) identical to the amino acid sequence of SEQ ID NO: 10 or 20. In certain embodiments, the anti-NKG2A antibody has an LC that comprises the amino acid sequence of SEQ ID NO: 10 or 20.

In some embodiments, the anti-NKG2A antibody comprises any of the above heavy chain sequences and any of the above light chain sequences.

In some embodiments, the anti-NKG2A antibody comprises the H-CDR1-3 and L-CDR1-3 amino acid sequences of:
a) SEQ ID NOs: 1-6, respectively; or
b) SEQ ID NOs: 11-16, respectively.

In some embodiments, the anti-NKG2A antibody comprises a VH and a VL that are at least 90% (e.g., at least 92%, at least 95%, at least 96%, at least 97%, at least 98%, or at least 99%) identical to the amino acid sequences of:
a) SEQ ID NOs: 7 and 8, respectively; or
b) SEQ ID NOs: 17 and 18, respectively.

In some embodiments, the anti-NKG2A antibody comprises a VH and a VL that comprise the amino acid sequences of:
a) SEQ ID NOs: 7 and 8, respectively; or
b) SEQ ID NOs: 17 and 18, respectively.

In some embodiments, the anti-NKG2A antibody comprises an HC and an LC that are at least 90% (e.g., at least 92%, at least 95%, at least 96%, at least 97%, at least 98%, or at least 99%) identical to the amino acid sequences of:
a) SEQ ID NOs: 9 and 10, respectively; or
b) SEQ ID NOs: 19 and 20, respectively.

In some embodiments, the anti-NKG2A antibody comprises an HC and an LC that comprise the amino acid sequences of:
a) SEQ ID NOs: 9 and 10, respectively; or
b) SEQ ID NOs: 19 and 20, respectively.

In some embodiments, an anti-NKG2A antibody or an antigen-binding portion thereof as described herein is an anti-NKG2A antibody or antigen-binding portion described in U.S. Provisional Patent Application 63/195,470, which is incorporated by reference in its entirety herein.

### Anti-PD-1 Antibodies

In some embodiments, a combination therapy or composition described herein comprises an anti-PD-1 antibody or an antigen-binding portion thereof. In certain embodiments, the anti-PD-1 antibody is nivolumab, pembrolizumab, cemiplimab, dostarlimab, mAb3 (comprising heavy and light chain amino acid sequences of SEQ ID NOs: 69 and 70, respectively), retifanlimab, or a variant of any of these, where the variant may contain, e.g., certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of the antibody.

In some embodiments, the anti-PD-1 antibody competes or cross-competes for binding to human PD-1 with, or binds to the same epitope of human PD-1 as, nivolumab, pembrolizumab, cemiplimab, dostarlimab, mAb3, or retifanlimab.

In some embodiments, the anti-PD-1 antibody comprises H-CDR1-3 and L-CDR1-3 of nivolumab, pembrolizumab, cemiplimab, dostarlimab, mAb3, or retifanlimab.

In some embodiments, the anti-PD-1 antibody comprises a VH and a VL at least 90% (e.g., at least 92%, at least 95%, at least 96%, at least 97%, at least 98%, or at least 99%) identical to the VH and VL of nivolumab, pembrolizumab, cemiplimab, dostarlimab, mAb3, or retifanlimab.

In some embodiments, the anti-PD-1 antibody comprises the VH and VL of nivolumab, pembrolizumab, cemiplimab, dostarlimab, mAb3, or retifanlimab.

In some embodiments, the anti-PD-1 antibody comprises an HC and an LC at least 90% (e.g., at least 92%, at least 95%, at least 96%, at least 97%, at least 98%, or at least 99%) identical to the HC and LC of nivolumab, pembrolizumab, cemiplimab, dostarlimab, mAb3, or retifanlimab.

In some embodiments, the anti-PD-1 antibody comprises the HC and LC of nivolumab, pembrolizumab, cemiplimab, dostarlimab, mAb3, or retifanlimab.

In some embodiments, the anti-PD-1 antibody comprises H-CDR1-3 comprising the amino acid sequences, respectively, of SEQ ID NOs: 21-23, 31-33, 41-43, 51-53, 61-63, or 71-73.

In some embodiments, the anti-PD-1 antibody has a VH that is at least 90% (e.g., at least 92%, at least 95%, at least 96%, at least 97%, at least 98%, or at least 99%) identical to the amino acid sequence of SEQ ID NO: 27, 37, 47, 57, 67, or 77. In certain embodiments, the anti-PD-1 antibody has a VH that comprises the amino acid sequence of SEQ ID NO: 27, 37, 47, 57, 67, or 77.

In some embodiments, the anti-PD-1 antibody has an HC that is at least 90% (e.g., at least 92%, at least 95%, at least 96%, at least 97%, at least 98%, or at least 99%) identical to the amino acid sequence of SEQ ID NO: 29, 39, 49, 59, 69, or 79.

In certain embodiments, the anti-PD-1 antibody has an HC that comprises the amino acid sequence of SEQ ID NO: 29, 39, 49, 59, 69, or 79.

In some embodiments, the anti-PD-1 antibody comprises L-CDR1-3 comprising the amino acid sequences, respectively, of SEQ ID NOs: 24-26, 34-36, 44-46, 54-56, 64-66, or 74-76.

In some embodiments, the anti-PD-1 antibody has a VL that is at least 90% (e.g., at least 92%, at least 95%, at least 96%, at least 97%, at least 98%, or at least 99%) identical to the amino acid sequence of SEQ ID NO: 28, 38, 48, 58, 68, or 78. In certain embodiments, the anti-PD-1 antibody has a VL that comprises the amino acid sequence of SEQ ID NO: 28, 38, 48, 58, 68, or 78.

In some embodiments, the anti-PD-1 antibody has an LC that is at least 90% (e.g., at least 92%, at least 95%, at least 96%, at least 97%, at least 98%, or at least 99%) identical to the amino acid sequence of SEQ ID NO: 30, 40, 50, 60, 70, or 80. In certain embodiments, the anti-PD-1 antibody has an LC that comprises the amino acid sequence of SEQ ID NO: 30, 40, 50, 60, 70, or 80.

In some embodiments, the anti-PD-1 antibody comprises any of the above heavy chain sequences and any of the above light chain sequences.

In some embodiments, the anti-PD-1 antibody comprises the H-CDR1-3 and L-CDR1-3 amino acid sequences of:
a) SEQ ID NOs: 21-26, respectively;
b) SEQ ID NOs: 31-36, respectively;
c) SEQ ID NOs: 41-46, respectively;
d) SEQ ID NOs: 51-56, respectively;
e) SEQ ID NOs: 61-66, respectively; or
f) SEQ ID NOs: 71-76, respectively.

In some embodiments, the anti-PD-1 antibody comprises a VH and a VL that are at least 90% (e.g., at least 92%, at least 95%, at least 96%, at least 97%, at least 98%, or at least 99%) identical to the amino acid sequences of:
a) SEQ ID NOs: 27 and 28, respectively;
b) SEQ ID NOs: 37 and 38, respectively;
c) SEQ ID NOs: 47 and 48, respectively;
d) SEQ ID NOs: 57 and 58, respectively;
e) SEQ ID NOs: 67 and 68, respectively; or
f) SEQ ID NOs: 77 and 78, respectively.

In some embodiments, the anti-PD-1 antibody comprises a VH and a VL that comprise the amino acid sequences of:
a) SEQ ID NOs: 27 and 28, respectively;
b) SEQ ID NOs: 37 and 38, respectively;
c) SEQ ID NOs: 47 and 48, respectively;
d) SEQ ID NOs: 57 and 58, respectively;
e) SEQ ID NOs: 67 and 68, respectively; or
f) SEQ ID NOs: 77 and 78, respectively.

In some embodiments, the anti-PD-1 antibody comprises an HC and an LC that are at least 90% (e.g., at least 92%, at least 95%, at least 96%, at least 97%, at least 98%, or at least 99%) identical to the amino acid sequences of:
a) SEQ ID NOs: 29 and 30, respectively;
b) SEQ ID NOs: 39 and 40, respectively;
c) SEQ ID NOs: 49 and 50, respectively;
d) SEQ ID NOs: 59 and 60, respectively;
e) SEQ ID NOs: 69 and 70, respectively; or
f) SEQ ID NOs: 79 and 80, respectively.

In some embodiments, the anti-PD-1 antibody comprises an HC and an LC that comprise the amino acid sequences of:
a) SEQ ID NOs: 29 and 30, respectively;
b) SEQ ID NOs: 39 and 40, respectively;
c) SEQ ID NOs: 49 and 50, respectively;
d) SEQ ID NOs: 59 and 60, respectively;
e) SEQ ID NOs: 69 and 70, respectively; or
f) SEQ ID NOs: 79 and 80, respectively.

In some embodiments, an anti-PD-1 antibody or an antigen-binding portion thereof as described herein is an anti-PD-1 antibody or antigen-binding portion described in PCT Patent Publication WO 2017/055547, which is incorporated by reference in its entirety herein.

### Anti-PD-L1 Antibodies

In some embodiments, a combination therapy or composition described herein comprises an anti-PD-L1 antibody or an antigen-binding portion thereof. In certain embodiments, the anti-PD-L1 antibody is atezolizumab, avelumab, durvalumab, or a variant of any of these, where the variant may contain, e.g., certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of the antibody.

In some embodiments, the anti-PD-L1 antibody competes or cross-competes for binding to human PD-L1 with, or binds to the same epitope of human PD-L1 as, atezolizumab, avelumab, or durvalumab.

In some embodiments, the anti-PD-L1 antibody comprises H-CDR1-3 and L-CDR1-3 of atezolizumab, avelumab, or durvalumab.

In some embodiments, the anti-PD-L1 antibody comprises a VH and a VL at least 90% (e.g., at least 92%, at least 95%, at least 96%, at least 97%, at least 98%, or at least 99%) identical to the VH and VL of atezolizumab, avelumab, or durvalumab.

In some embodiments, the anti-PD-L1 antibody comprises the VH and VL of atezolizumab, avelumab, or durvalumab.

In some embodiments, the anti-PD-L1 antibody comprises an HC and an LC at least 90% (e.g., at least 92%, at least 95%, at least 96%, at least 97%, at least 98%, or at least 99%) identical to the HC and LC of atezolizumab, avelumab, or durvalumab.

In some embodiments, the anti-PD-L1 antibody comprises the HC and LC of atezolizumab, avelumab, or durvalumab.

In some embodiments, the anti-PD-L1 antibody comprises H-CDR1-3 comprising the amino acid sequences, respectively, of SEQ ID NOs: 81-83, 91-93, or 101-103.

In some embodiments, the anti-PD-L1 antibody has a VH that is at least 90% (e.g., at least 92%, at least 95%, at least 96%, at least 97%, at least 98%, or at least 99%) identical to the amino acid sequence of SEQ ID NO: 87, 97, or 107. In certain embodiments, the anti-PD-L1 antibody has a VH that comprises the amino acid sequence of SEQ ID NO: 87, 97, or 107.

In some embodiments, the anti-PD-L1 antibody has an HC that is at least 90% (e.g., at least 92%, at least 95%, at least 96%, at least 97%, at least 98%, or at least 99%) identical to the amino acid sequence of SEQ ID NO: 89, 99, or 109. In certain embodiments, the anti-PD-L1 antibody has an HC that comprises the amino acid sequence of SEQ ID NO: 89, 99, or 109.

In some embodiments, the anti-PD-L1 antibody comprises L-CDR1-3 comprising the amino acid sequences, respectively, of SEQ ID NOs: 84-86, 94-96, or 104-106.

In some embodiments, the anti-PD-L1 antibody has a VL that is at least 90% (e.g., at least 92%, at least 95%, at least 96%, at least 97%, at least 98%, or at least 99%) identical to the amino acid sequence of SEQ ID NO: 88, 98, or 108. In certain embodiments, the anti-PD-L1 antibody has a VL that comprises the amino acid sequence of SEQ ID NO: 88, 98, or 108.

In some embodiments, the anti-PD-L1 antibody has an LC that is at least 90% (e.g., at least 92%, at least 95%, at least 96%, at least 97%, at least 98%, or at least 99%) identical to the amino acid sequence of SEQ ID NO: 90, 100, or 110. In certain embodiments, the anti-PD-L1 antibody has an LC that comprises the amino acid sequence of SEQ ID NO: 90, 100, or 110.

In some embodiments, the anti-PD-L1 antibody comprises any of the above heavy chain sequences and any of the above light chain sequences.

In some embodiments, the anti-PD-L1 antibody comprises the H-CDR1-3 and L-CDR1-3 amino acid sequences of:
a) SEQ ID NOs: 81-86, respectively;
b) SEQ ID NOs: 91-96, respectively; or
c) SEQ ID NOs: 101-106, respectively.

In some embodiments, the anti-PD-L1 antibody comprises a VH and a VL that are at least 90% (e.g., at least 92%, at least 95%, at least 96%, at least 97%, at least 98%, or at least 99%) identical to the amino acid sequences of:
a) SEQ ID NOs: 87 and 88, respectively;
b) SEQ ID NOs: 97 and 98, respectively; or
c) SEQ ID NOs: 107 and 108, respectively.

In some embodiments, the anti-PD-L1 antibody comprises a VH and a VL that comprise the amino acid sequences of:
a) SEQ ID NOs: 87 and 88, respectively;
b) SEQ ID NOs: 97 and 98, respectively; or
c) SEQ ID NOs: 107 and 108, respectively.

In some embodiments, the anti-PD-L1 antibody comprises an HC and an LC that are at least 90% (e.g., at least 92%, at least 95%, at least 96%, at least 97%, at least 98%, or at least 99%) identical to the amino acid sequences of:
a) SEQ ID NOs: 89 and 90, respectively;
b) SEQ ID NOs: 99 and 100, respectively; or
c) SEQ ID NOs: 109 and 110, respectively.

In some embodiments, the anti-PD-L1 antibody comprises an HC and an LC that comprise the amino acid sequences of:
a) SEQ ID NOs: 89 and 90, respectively;
b) SEQ ID NOs: 99 and 100, respectively; or
c) SEQ ID NOs: 109 and 110, respectively.

### Anti-EGFR Antibodies

In some embodiments, a combination therapy or composition described herein comprises an anti-EGFR antibody or an antigen-binding portion thereof. In certain embodiments, the anti-EGFR antibody is cetuximab, panitumumab, futuximab, modotuximab, or a variant of any of these, where the variant may contain, e.g., certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of the antibody.

In some embodiments, the anti-EGFR antibody competes or cross-competes for binding to human EGFR with, or binds to the same epitope of human EGFR as, cetuximab, panitumumab, futuximab, or modotuximab.

In some embodiments, the anti-EGFR antibody comprises H-CDR1-3 and L-CDR1-3 of cetuximab, panitumumab, futuximab, or modotuximab.

In some embodiments, the anti-EGFR antibody comprises a VH and a VL at least 90% (e.g., at least 92%, at least 95%, at least 96%, at least 97%, at least 98%, or at least 99%) identical to the VH and VL of cetuximab, panitumumab, futuximab, or modotuximab.

In some embodiments, the anti-EGFR antibody comprises the VH and VL of cetuximab, panitumumab, futuximab, or modotuximab.

In some embodiments, the anti-EGFR antibody comprises an HC and an LC at least 90% (e.g., at least 92%, at least 95%, at least 96%, at least 97%, at least 98%, or at least 99%) identical to the HC and LC of cetuximab, panitumumab, futuximab, or modotuximab.

In some embodiments, the anti-EGFR antibody comprises the HC and LC of cetuximab, panitumumab, futuximab, or modotuximab.

In some embodiments, the anti-EGFR antibody comprises H-CDR1-3 comprising the amino acid sequences, respectively, of SEQ ID NOs: 111-113, 121-123, 131-133, or 141-143.

In some embodiments, the anti-EGFR antibody has a VH that is at least 90% (e.g., at least 92%, at least 95%, at least 96%, at least 97%, at least 98%, or at least 99%) identical to the amino acid sequence of SEQ ID NO: 117, 127, 137, or 147. In certain embodiments, the anti-EGFR antibody has a VH that comprises the amino acid sequence of SEQ ID NO: 117, 127, 137, or 147.

In some embodiments, the anti-EGFR antibody has an HC that is at least 90% (e.g., at least 92%, at least 95%, at least 96%, at least 97%, at least 98%, or at least 99%) identical to the amino acid sequence of SEQ ID NO: 119, 129, 139, or 149. In certain embodiments, the anti-EGFR antibody has an HC that comprises the amino acid sequence of SEQ ID NO: 119, 129, 139, or 149.

In some embodiments, the anti-EGFR antibody comprises L-CDR1-3 comprising the amino acid sequences, respectively, of SEQ ID NOs: 114-116, 124-126, 134-136, or 144-146.

In some embodiments, the anti-EGFR antibody has a VL that is at least 90% (e.g., at least 92%, at least 95%, at least 96%, at least 97%, at least 98%, or at least 99%) identical to the amino acid sequence of SEQ ID NO: 118, 128, 138, or 148. In certain embodiments, the anti-EGFR antibody has a VL that comprises the amino acid sequence of SEQ ID NO: 118, 128, 138, or 148.

In some embodiments, the anti-EGFR antibody has an LC that is at least 90% (e.g., at least 92%, at least 95%, at least 96%, at least 97%, at least 98%, or at least 99%) identical to the amino acid sequence of SEQ ID NO: 120, 130, 140, or 150. In certain embodiments, the anti-EGFR antibody has an LC that comprises the amino acid sequence of SEQ ID NO: 120, 130, 140, or 150.

In some embodiments, the anti-EGFR antibody comprises any of the above heavy chain sequences and any of the above light chain sequences.

In some embodiments, the anti-EGFR antibody comprises the H-CDR1-3 and L-CDR1-3 amino acid sequences of:
a) SEQ ID NOs: 111-116, respectively;
b) SEQ ID NOs: 121-126, respectively;
c) SEQ ID NOs: 131-136, respectively; or
d) SEQ ID NOs: 141-146, respectively.

In some embodiments, the anti-EGFR antibody comprises a VH and a VL that are at least 90% (e.g., at least 92%, at least 95%, at least 96%, at least 97%, at least 98%, or at least 99%) identical to the amino acid sequences of:
a) SEQ ID NOs: 117 and 118, respectively;
b) SEQ ID NOs: 127 and 128, respectively;
c) SEQ ID NOs: 137 and 138, respectively; or
d) SEQ ID NOs: 147 and 148, respectively.

In some embodiments, the anti-EGFR antibody comprises a VH and a VL that comprise the amino acid sequences of:
a) SEQ ID NOs: 117 and 118, respectively;
b) SEQ ID NOs: 127 and 128, respectively;
c) SEQ ID NOs: 137 and 138, respectively; or
d) SEQ ID NOs: 147 and 148, respectively.

In some embodiments, the anti-EGFR antibody comprises an HC and an LC that are at least 90% (e.g., at least 92%, at least 95%, at least 96%, at least 97%, at least 98%, or at least 99%) identical to the amino acid sequences of: a) SEQ ID NOs: 119 and 120, respectively;
b) SEQ ID NOs: 129 and 130, respectively;
c) SEQ ID NOs: 139 and 140, respectively; or
d) SEQ ID NOs: 149 and 150, respectively.

In some embodiments, the anti-EGFR antibody comprises an HC and an LC that comprise the amino acid sequences of:
a) SEQ ID NOs: 119 and 120, respectively;
b) SEQ ID NOs: 129 and 130, respectively;
c) SEQ ID NOs: 139 and 140, respectively; or
d) SEQ ID NOs: 149 and 150, respectively.

In some embodiments, an anti-EGFR combination of futuximab and modotuximab (e.g., in a 1:1 ratio), or a combination comprising a variant of one or both antibodies, may be used where the combination therapy or composition of the present disclosure calls for an “anti-EGFR antibody.” In some embodiments, an anti-EGFR antibody or an anti-EGFR combination is referred to as an “anti-EGFR component.”

In some embodiments, the anti-EGFR combination comprises first and second antibodies that compete or cross-compete for binding to human EGFR with, or bind to the same epitope of human EGFR as, futuximab and modotuximab, respectively.

In some embodiments, the anti-EGFR combination comprises first and second antibodies that comprise the H-CDR1-3 and L-CDR1-3 of futuximab and modotuximab, respectively.

In some embodiments, the anti-EGFR combination comprises first and second antibodies that comprise a VH and a VL at least 90% (e.g., at least 92%, at least 95%, at least 96%, at least 97%, at least 98%, or at least 99%) identical to the VH and VL of futuximab and modotuximab, respectively.

In some embodiments, the anti-EGFR combination comprises first and second antibodies that comprise the VH and VL of futuximab and modotuximab, respectively.

In some embodiments, the anti-EGFR combination comprises first and second antibodies that comprise an HC and an LC at least 90% (e.g., at least 92%, at least 95%, at least 96%, at least 97%, at least 98%, or at least 99%) identical to the HC and LC of futuximab and modotuximab, respectively.

In some embodiments, the anti-EGFR combination comprises first and second antibodies that comprise the HC and LC of futuximab and modotuximab, respectively.

In some embodiments, the anti-EGFR combination comprises first and second antibodies with H-CDR1-3 comprising the amino acid sequences, respectively, of SEQ ID NOs: 131-133 and SEQ ID NOs: 141-143, respectively.

In some embodiments, the anti-EGFR combination comprises first and second antibodies that comprise a VH that is at least 90% (e.g., at least 92%, at least 95%, at least 96%, at least 97%, at least 98%, or at least 99%) identical to the amino acid sequence of SEQ ID NO: 137 and the amino acid sequence of SEQ ID NO: 147, respectively. In certain embodiments, the anti-EGFR combination comprises a first antibody that has a VH that comprises the amino acid sequence of SEQ ID NO: 137, and a second antibody that has a VH that comprises the amino acid sequence of SEQ ID NO: 147.

In some embodiments, the anti-EGFR combination comprises first and second antibodies that comprise an HC that is at least 90% (e.g., at least 92%, at least 95%, at least 96%, at least 97%, at least 98%, or at least 99%) identical to the amino acid sequence of SEQ ID NO: 139 and the amino acid sequence of SEQ ID NO: 149, respectively. In certain embodiments, the anti-EGFR combination comprises a first antibody that has an HC that comprises the amino acid sequence of SEQ ID NO: 139, and a second antibody that has an HC that comprises the amino acid sequence of SEQ ID NO: 149.

In some embodiments, the anti-EGFR combination comprises first and second antibodies with L-CDR1-3 comprising the amino acid sequences, respectively, of SEQ ID NOs: 134-136 and SEQ ID NOs: 144-146, respectively.

In some embodiments, the anti-EGFR combination comprises first and second antibodies that comprise a VL that is at least 90% (e.g., at least 92%, at least 95%, at least 96%, at least 97%, at least 98%, or at least 99%) identical to the amino acid sequence of SEQ ID NO: 138 and the amino acid sequence of SEQ ID NO: 148, respectively. In certain embodiments, the anti-EGFR combination comprises a first antibody that has a VL that comprises the amino acid sequence of SEQ ID NO: 138, and a second antibody that has a VL that comprises the amino acid sequence of SEQ ID NO: 148.

In some embodiments, the anti-EGFR combination comprises first and second antibodies that comprise an LC that is at least 90% (e.g., at least 92%, at least 95%, at least 96%, at least 97%, at least 98%, or at least 99%) identical to the amino acid sequence of SEQ ID NO: 140 and the amino acid sequence of SEQ ID NO: 150, respectively. In certain embodiments, the anti-EGFR combination comprises a first antibody that has an LC that comprises the amino acid sequence of SEQ ID NO: 140 and a second antibody that has an LC that comprises the amino acid sequence of SEQ ID NO: 150.

In some embodiments, the anti-EGFR combination comprises first and second antibodies with any of the above heavy chain sequences and any of the above light chain sequences for the first and second antibodies, respectively.

In some embodiments, the anti-EGFR combination comprises:
a) a first antibody comprising the H-CDR1-3 and L-CDR1-3 amino acid sequences of SEQ ID NOs: 131-136, respectively; and
b) a second antibody comprising the H-CDR1-3 and L-CDR1-3 amino acid sequences of SEQ ID NOs: 141-146, respectively.

In some embodiments, the anti-EGFR combination comprises:
a) a first antibody comprising a VH and a VL that are at least 90% (e.g., at least 92%, at least 95%, at least 96%, at least 97%, at least 98%, or at least 99%) identical to the amino acid sequences of SEQ ID NOs: 137 and 138, respectively; and
b) a second antibody comprising a VH and a VL that are at least 90% (e.g., at least 92%, at least 95%, at least 96%, at least 97%, at least 98%, or at least 99%) identical to the amino acid sequences of SEQ ID NOs: 147 and 148, respectively.

In some embodiments, the anti-EGFR combination comprises:
a) a first antibody comprising a VH and a VL that comprise the amino acid sequences of SEQ ID NOs: 137 and 138, respectively; and
b) a second antibody comprising a VH and a VL that comprise the amino acid sequences of SEQ ID NOs: 147 and 148, respectively.

In some embodiments, the anti-EGFR combination comprises:
a) a first antibody comprising an HC and an LC that are at least 90% (e.g., at least 92%, at least 95%, at least 96%, at least 97%, at least 98%, or at least 99%) identical to the amino acid sequences of SEQ ID NOs: 139 and 140, respectively; and
b) a second antibody comprising an HC and an LC that are at least 90% (e.g., at least 92%, at least 95%, at least 96%, at least 97%, at least 98%, or at least 99%) identical to the amino acid sequences of SEQ ID NOs: 149 and 150, respectively.

In some embodiments, the anti-EGFR combination comprises:
a) a first antibody comprising an HC and an LC that comprise the amino acid sequences of SEQ ID NOs: 139 and 140, respectively; and
b) a second antibody comprising an HC and an LC that comprise the amino acid sequences of SEQ ID NOs: 149 and 150, respectively.

In some embodiments, an anti-EGFR antibody or an antigen-binding portion thereof or an anti-EGFR combination as described herein is an anti-EGFR antibody or antigen-binding portion or combination described in PCT Patent Publication WO 2008/104183, which is incorporated by reference in its entirety herein.

### Anti-HER2 Antibodies

In some embodiments, a combination therapy or composition described herein comprises an anti-HER2 antibody or an antigen-binding portion thereof. In certain embodiments, the anti-HER2 antibody is trastuzumab or margetuximab, or a variant of either of these, where the variant may contain, e.g., certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of the antibody.

In some embodiments, the anti-HER2 antibody competes or cross-competes for binding to human HER2 with, or binds to the same epitope of human HER2 as, trastuzumab or margetuximab.

In some embodiments, the anti-HER2 antibody comprises H-CDR1-3 and L-CDR1-3 of trastuzumab or margetuximab.

In some embodiments, the anti-HER2 antibody comprises a VH and a VL at least 90% (e.g., at least 92%, at least 95%, at least 96%, at least 97%, at least 98%, or at least 99%) identical to the VH and VL of trastuzumab or margetuximab.

In some embodiments, the anti-HER2 antibody comprises the VH and VL of trastuzumab or margetuximab.

In some embodiments, the anti-HER2 antibody comprises an HC and an LC at least 90% (e.g., at least 92%, at least 95%, at least 96%, at least 97%, at least 98%, or at least 99%) identical to the HC and LC of trastuzumab or margetuximab.

In some embodiments, the anti-HER2 antibody comprises the HC and LC of trastuzumab or margetuximab.

In some embodiments, the anti-HER2 antibody comprises H-CDR1-3 comprising the amino acid sequences, respectively, of SEQ ID NOs: 151-153 or 161-163.

In some embodiments, the anti-HER2 antibody has a VH that is at least 90% (e.g., at least 92%, at least 95%, at least 96%, at least 97%, at least 98%, or at least 99%) identical to the amino acid sequence of SEQ ID NO: 157 or 167. In certain embodiments, the anti-HER2 antibody has a VH that comprises the amino acid sequence of SEQ ID NO: 157 or 167.

In some embodiments, the anti-HER2 antibody has an HC that is at least 90% (e.g., at least 92%, at least 95%, at least 96%, at least 97%, at least 98%, or at least 99%) identical to the amino acid sequence of SEQ ID NO: 159 or 169. In certain embodiments, the anti-HER2 antibody has an HC that comprises the amino acid sequence of SEQ ID NO: 159 or 169.

In some embodiments, the anti-HER2 antibody comprises L-CDR1-3 comprising the amino acid sequences, respectively, of SEQ ID NOs: 154-156 or 164-166.

In some embodiments, the anti-HER2 antibody has a VL that is at least 90% (e.g., at least 92%, at least 95%, at least 96%, at least 97%, at least 98%, or at least 99%) identical to the amino acid sequence of SEQ ID NO: 158 or 168. In certain embodiments, the anti-HER2 antibody has a VL that comprises the amino acid sequence of SEQ ID NO: 158 or 168.

In some embodiments, the anti-HER2 antibody has an LC that is at least 90% (e.g., at least 92%, at least 95%, at least 96%, at least 97%, at least 98%, or at least 99%) identical to the amino acid sequence of SEQ ID NO: 160 or 170. In certain embodiments, the anti-HER2 antibody has an LC that comprises the amino acid sequence of SEQ ID NO: 160 or 170.

In some embodiments, the anti-HER2 antibody comprises any of the above heavy chain sequences and any of the above light chain sequences.

In some embodiments, the anti-HER2 antibody comprises the H-CDR1-3 and L-CDR1-3 amino acid sequences of:
a) SEQ ID NOs: 151-156, respectively; or
b) SEQ ID NOs: 161-166, respectively.

In some embodiments, the anti-HER2 antibody comprises a VH and a VL that are at least 90% (e.g., at least 92%, at least 95%, at least 96%, at least 97%, at least 98%, or at least 99%) identical to the amino acid sequences of:
a) SEQ ID NOs: 157 and 158, respectively; or
b) SEQ ID NOs: 167 and 168, respectively.

In some embodiments, the anti-HER2 antibody comprises a VH and a VL that comprise the amino acid sequences of:
a) SEQ ID NOs: 157 and 158, respectively; or
b) SEQ ID NOs: 167 and 168, respectively.

In some embodiments, the anti-HER2 antibody comprises an HC and an LC that are at least 90% (e.g., at least 92%, at least 95%, at least 96%, at least 97%, at least 98%, or at least 99%) identical to the amino acid sequences of:
a) SEQ ID NOs: 159 and 160, respectively; or
b) SEQ ID NOs: 169 and 170, respectively.

In some embodiments, the anti-HER2 antibody comprises an HC and an LC that comprise the amino acid sequences of:
a) SEQ ID NOs: 159 and 160, respectively; or
b) SEQ ID NOs: 169 and 170, respectively.

In some embodiments, an anti-HER2 antibody drug conjugate (ADC), may be used where the combination therapy or composition of the present disclosure calls for an “anti-HER2 antibody.” In certain embodiments, the ADC comprises an anti-HER2 antibody described herein and DXd or DM1. In particular embodiments, the ADC is trastuzumab dexrutecan or trastuzumab emtansine.

The class of an antibody described herein may be changed or switched with another class or subclass. In some embodiments of the present disclosure, a nucleic acid molecule encoding VL or VH is isolated using methods well known in the art such that it does not include nucleic acid sequences encoding CL or CH, respectively. The nucleic acid molecules encoding VL or VH then are operatively linked to a nucleic acid sequence encoding a CL or CH, respectively, from a different class of immunoglobulin molecule. This may be achieved using a vector or nucleic acid molecule that comprises a CL or CH sequence, as described above. For example, an antibody that was originally IgM may be class switched to IgG. Further, the class switching may be used to convert one IgG subclass to another, e.g., from IgG₁ to IgG₂. A κ light chain constant region can be changed, e.g., to a λ light chain constant region, or vice-versa. An exemplary method for producing an antibody described herein with a desired Ig isotype comprises the steps of isolating a nucleic acid molecule encoding the heavy chain of an antibody and a nucleic acid molecule encoding the light chain of an antibody, obtaining the variable domain of the heavy chain, ligating a coding sequence for the variable domain of the heavy chain with a coding sequence for the constant region of a heavy chain of the desired isotype, expressing the light chain and the heavy chain encoded by the ligated sequence in a cell, and collecting the antibody with the desired isotype.

An antibody described herein can be an IgG, an IgM, an IgE, an IgA, or an IgD molecule, but is typically of the IgG isotype, e.g., of IgG subclass IgG₁, IgG₂, or IgG2b, IgG₃ or IgG₄. In some embodiments, the antibody is of the isotype subclass IgG₁.

In some embodiments, the antibody may comprise at least one mutation in the Fc region. A number of different Fc mutations are known, where these mutations alter the antibody's effector function. For example, in some embodiments, the antibody comprises at least one mutation in the Fc region that reduces effector function, e.g., mutations at one or more of positions 228, 233, 234 and 235, where amino acid positions are numbered according to Eu numbering.

In some embodiments, e.g., where the antibody is of the IgG₁ subclass, one or both of the amino acid residues at positions 234 and 235 may be mutated, for example from Leu to Ala (L234A/L235A). These mutations reduce effector function of the Fc region of IgG₁ antibodies. The amino acid positions are numbered according to the Eu numbering scheme.

In some embodiments, e.g., where the antibody is of the IgG₄ subclass, it may comprise the mutation S228P, where the amino acid position is numbered according to the Eu numbering scheme. This mutation is known to reduce undesired Fab arm exchange.

In certain embodiments, an antibody or antigen-binding portion thereof described herein may be part of a larger immunoadhesin molecule, formed by covalent or noncovalent association of the antibody or antibody portion with one or more other proteins or peptides. Examples of such immunoadhesin molecules include use of the streptavidin core region to make a tetrameric scFv molecule (Kipriyanov et al., Human Antibodies and Hybridomas (1995) 6:93-101) and use of a cysteine residue, a marker peptide and a C-terminal polyhistidine tag to make bivalent and biotinylated scFv molecules (Kipriyanov et al., Mol. Immunol. (1994) 31:1047-58). Other examples include where one or more CDRs from an antibody are incorporated into a molecule either covalently or noncovalently to make it an immunoadhesin that specifically binds to an antigen of interest. In such embodiments, the CDR(s) may be incorporated as part of a larger polypeptide chain, may be covalently linked to another polypeptide chain, or may be incorporated noncovalently.

In another aspect, a fusion antibody or immunoadhesin may be made that comprises all or a portion of an antibody described herein linked to another polypeptide. In certain embodiments, only the variable domains of the antibody are linked to the polypeptide. In certain embodiments, the VH domain of an antibody is linked to a first polypeptide, while the VL domain of an antibody is linked to a second polypeptide that associates with the first polypeptide in a manner such that the VH and VL domains can interact with one another to form an antigen-binding site. In some embodiments, the VH domain is separated from the VL domain by a linker such that the VH and VL domains can interact with one another (e.g., single-chain antibodies). The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bi-specific antibody.

To create a single chain antibody (scFv), the VH- and VL-encoding DNA fragments are operatively linked to another fragment encoding a flexible linker, e.g., encoding the amino acid sequence (Gly₄-Ser)₃ (SEQ ID NO: 176), such that the VH and VL sequences can be expressed as a contiguous single-chain protein, with the VL and VH domains joined by the flexible linker. See, e.g., Bird et al., Science (1988) 242:423-6; Huston et al., Proc. Natl. Acad. Sci. USA (1988) 85:5879-83; and McCafferty et al., Nature (1990) 348:552-4. The single chain antibody may be monovalent, if only a single VH and VL are used; bivalent, if two VH and VL are used; or polyvalent, if more than two VH and VL are used. Multispecific or polyvalent antibodies may be generated that bind specifically to the targets described herein, for instance.

In other embodiments, other modified antibodies may be prepared using antibody-encoding nucleic acid molecules. For instance, “kappa bodies” (Ill et al., Protein Eng. (1997) 10:949-57), “minibodies” (Martin et al., EMBO J. (1994) 13:5303-9), “diabodies” (Holliger et al., Proc. Natl. Acad. Sci. USA (1993) 90:6444-8), or “Janusins” (Traunecker et al., EMBO J. (1991) 10:3655-9 and Traunecker et al., Int. J. Cancer (Suppl.) (1992) 7:51-2) may be prepared using standard molecular biological techniques following the teachings of the specification.

An antibody or antigen-binding portion described herein can be derivatized or linked to another molecule (e.g., another peptide or protein). In general, the antibodies or portions thereof are derivatized such that target binding is not affected adversely by the derivatization or labeling. Accordingly, the antibodies and antibody portions of the present disclosure are intended to include both intact and modified forms of the antibodies described herein. For example, an antibody or antibody portion of the present disclosure can be functionally linked (by chemical coupling, genetic fusion, noncovalent association or otherwise) to one or more other molecular entities, such as another antibody (e.g., a bi-specific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bi-specific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available, e.g., from Pierce Chemical Company, Rockford, IL.

An antibody or antigen-binding portion can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody or antigen-binding portion described herein may also be labeled. As used herein, the terms “label” or “labeled” refer to incorporation of another molecule in the antibody. In some embodiments, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In some embodiments, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In some embodiments, an antibody or antigen-binding portion described herein may be conjugated to a cytotoxic agent to form an immunoconjugate. In some embodiments, an antibody or antigen-binding portion according to the present disclosure may be conjugated to a radioisotope.

In certain embodiments, the antibodies described herein may be present in a neutral form (including zwitterionic forms) or as a positively or negatively-charged species. In some embodiments, the antibodies may be complexed with a counterion to form a pharmaceutically acceptable salt.

### Combination Therapies

In addition to monotherapies comprising an anti-NKG2A antibody, the present disclosure provides a combination therapy that comprises an anti-NKG2A antibody or antigen-binding portion thereof in combination with (1) an anti-PD-1 antibody, (2) an anti-PD-L1 antibody, (3) an anti-EGFR antibody, (4) an anti-HER2 antibody, or (5) any combination thereof. In certain embodiments, the combination therapy comprises:
a) an anti-NKG2A antibody or an antigen-binding portion thereof, and
b) an anti-PD-1 or anti-PD-L1 antibody or an antigen-binding portion thereof.
   In certain embodiments, the combination therapy comprises:
a) an anti-NKG2A antibody or an antigen-binding portion thereof, and
b) an anti-EGFR or anti-HER2 antibody or an antigen-binding portion thereof.
   In certain embodiments, the combination therapy comprises:
a) an anti-NKG2A antibody or an antigen-binding portion thereof,
b) an anti-PD-1 or anti-PD-L1 antibody or an antigen-binding portion thereof, and
c) an anti-EGFR or anti-HER2 antibody or an antigen-binding portion thereof.
   The anti-NKG2A antibody, anti-PD-1 antibody, anti-PD-L1 antibody, anti-EGFR antibody, and/or anti-HER2 antibody may be an antibody to said target as described herein. The combination therapy may take the form of, e.g., a method for treatment using said antibodies or antigen-binding portions or a pharmaceutical composition comprising said antibodies or antigen-binding portions.

In some embodiments, the combination therapy or composition of the present disclosure comprises:
a) an anti-NKG2A antibody or an antigen-binding portion thereof as described herein (e.g., mAb1 or mAb2), and
b) an anti-PD-1 antibody or an antigen-binding portion thereof as described herein (e.g., nivolumab, pembrolizumab, cemiplimab, dostarlimab, mAb3, or retifanlimab).

In some embodiments, the combination therapy or composition of the present disclosure comprises:
a) an anti-NKG2A antibody or an antigen-binding portion thereof as described herein (e.g., mAb1 or mAb2),
b) an anti-PD-L1 antibody or an antigen-binding portion thereof as described herein (e.g., atezolizumab, avelumab, or durvalumab).

In some embodiments, the combination therapy or composition of the present disclosure comprises:
a) an anti-NKG2A antibody or an antigen-binding portion thereof as described herein (e.g., mAb1 or mAb2),
b) an anti-EGFR antibody or an antigen-binding portion thereof as described herein (e.g., cetuximab, panitumumab, futuximab, modotuximab, or futuximab+modotuximab).

In some embodiments, the combination therapy or composition of the present disclosure comprises:
a) an anti-NKG2A antibody or an antigen-binding portion thereof as described herein (e.g., mAb1 or mAb2),
b) an anti-HER2 antibody or an antigen-binding portion thereof as described herein (e.g., trastuzumab, margetuximab, trastuzumab dexrutecan, or trastuzumab emtansine).

In some embodiments, the combination therapy or composition of the present disclosure comprises:
a) an anti-NKG2A antibody or an antigen-binding portion thereof as described herein (e.g., mAb1 or mAb2),
b) an anti-PD-1 antibody or an antigen-binding portion thereof as described herein (e.g., nivolumab, pembrolizumab, cemiplimab, dostarlimab, mAb3, or retifanlimab), and
c) an anti-EGFR antibody or an antigen-binding portion thereof as described herein (e.g., cetuximab, panitumumab, futuximab, modotuximab, or futuximab+modotuximab).

In some embodiments, the combination therapy or composition of the present disclosure comprises:
a) an anti-NKG2A antibody or an antigen-binding portion thereof as described herein (e.g., mAb1 or mAb2),
b) an anti-PD-L1 antibody or an antigen-binding portion thereof as described herein (e.g., atezolizumab, avelumab, or durvalumab), and
c) an anti-EGFR antibody or an antigen-binding portion thereof as described herein (e.g., cetuximab, panitumumab, futuximab, modotuximab, or futuximab+modotuximab).

In some embodiments, the combination therapy or composition of the present disclosure comprises:
a) an anti-NKG2A antibody or an antigen-binding portion thereof as described herein (e.g., mAb1 or mAb2),
b) an anti-PD-1 antibody or an antigen-binding portion thereof as described herein (e.g., nivolumab, pembrolizumab, cemiplimab, dostarlimab, mAb3, or retifanlimab), and
c) an anti-HER2 antibody or an antigen-binding portion thereof as described herein (e.g., trastuzumab, margetuximab, trastuzumab dexrutecan, or trastuzumab emtansine).

In some embodiments, the combination therapy or composition of the present disclosure comprises:
a) an anti-NKG2A antibody or an antigen-binding portion thereof as described herein (e.g., mAb1 or mAb2),
b) an anti-PD-L1 antibody or an antigen-binding portion thereof as described herein (e.g., atezolizumab, avelumab, or durvalumab), and
c) an anti-HER2 antibody or an antigen-binding portion thereof as described herein (e.g., trastuzumab, margetuximab, trastuzumab dexrutecan, or trastuzumab emtansine).

In some embodiments, the combination therapy or composition of the present disclosure comprises:
(1) mAb1, and (2) mAb3;
(1) mAb1, and (2) pembrolizumab;
(1) mAb1, and (2) cetuximab;
(1) mAb1, and (2) futuximab+modotuximab;
(1) mAb1, and (2) avelumab;
(1) mAb1, (2) mAb3, and (3) futuximab+modotuximab;
(1) mAb1, (2) mAb3, and (3) margetuximab;
(1) mAb1, (2) retifanlimab, and (3) margetuximab;
(1) mAb, (2) pembrolizumab, and (3) trastuzumab;
(1) mAb1, (2) nivolumab, and (3) trastuzumab;
(1) mAb, (2) pembrolizumab, and (3) futuximab+modotuximab;
(1) mAb1, (2) nivolumab, and (3) futuximab+modotuximab;
(1) mAb1, (2) nivolumab, and (3) cetuximab; or
(1) mAb1, (2) pembrolizumab, and (3) cetuximab.

In some embodiments, the combination therapy or composition comprises antibodies or antigen-binding portions with the six CDRs, VH and VL, or HO and LC of said antibodies.

Sequences for the above-referenced antibodies may be, e.g., those found in Table 4. The SEQ ID NOs for these sequences are assigned as shown in Table 1 below:

TABLE 1
Antibody Sequence Identifiers

| | SEQ ID NO | | | | | | | | | |
|---|---|---|---|---|---|---|---|---|---|---|
| | H- | H- | H- | L- | L- | L- | | | | |
| Antibody | CDR1 | CDR2 | CDR3 | CDR1 | CDR2 | CDR3 | VH | VL | HC | LC |

NKG2A

| | | | | | | | | | | |
|---|---|---|---|---|---|---|---|---|---|---|
| mAb1 | 1 | 2 | 3 | 4 | 5 | 6 | 7 | 8 | 9 | 10 |
| mAb2 | 11 | 12 | 13 | 14 | 15 | 16 | 17 | 18 | 19 | 20 |

PD-1

| | | | | | | | | | | |
|---|---|---|---|---|---|---|---|---|---|---|
| nivolumab | 21 | 22 | 23 | 24 | 25 | 26 | 27 | 28 | 29 | 30 |
| pembrolizumab | 31 | 32 | 33 | 34 | 35 | 36 | 37 | 38 | 39 | 40 |
| cemiplimab | 41 | 42 | 43 | 44 | 45 | 46 | 47 | 48 | 49 | 50 |
| dostarlimab | 51 | 52 | 53 | 54 | 55 | 56 | 57 | 58 | 59 | 60 |
| mAb3 | 61 | 62 | 63 | 64 | 65 | 66 | 67 | 68 | 69 | 70 |
| retifanlimab | 71 | 72 | 73 | 74 | 75 | 76 | 77 | 78 | 79 | 80 |

PD-L1

| | | | | | | | | | | |
|---|---|---|---|---|---|---|---|---|---|---|
| atezolizumab | 81 | 82 | 83 | 84 | 85 | 86 | 87 | 88 | 89 | 90 |
| avelumab | 91 | 92 | 93 | 94 | 95 | 96 | 97 | 98 | 99 | 100 |
| durvalumab | 101 | 102 | 103 | 104 | 105 | 106 | 107 | 108 | 109 | 110 |

EGFR

| | | | | | | | | | | |
|---|---|---|---|---|---|---|---|---|---|---|
| cetuximab | 111 | 112 | 113 | 114 | 115 | 116 | 117 | 118 | 119 | 120 |
| panitumumab | 121 | 122 | 123 | 124 | 125 | 126 | 127 | 128 | 129 | 130 |
| futuximab | 131 | 132 | 133 | 134 | 135 | 136 | 137 | 138 | 139 | 140 |
| modotuximab | 141 | 142 | 143 | 144 | 145 | 146 | 147 | 148 | 149 | 150 |

HER2

| | | | | | | | | | | |
|---|---|---|---|---|---|---|---|---|---|---|
| trastuzumab | 151 | 152 | 153 | 154 | 155 | 156 | 157 | 158 | 159 | 160 |
| margetuximab | 161 | 162 | 163 | 164 | 165 | 166 | 167 | 168 | 169 | 170 |

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 61-66, respectively; and an anti-EGFR combination comprising an antibody or an antigen-binding portion thereof comprising the H-CDR1-3 and L-CDR1-3 amino acid sequences of SEQ ID NOs: 131-136, respectively, and an antibody or an antigen-binding portion thereof comprising the H-CDR1-3 and L-CDR1-3 amino acid sequences of SEQ ID NOs: 141-146, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 67 and 68, respectively; and an anti-EGFR combination comprising an antibody or an antigen-binding portion thereof comprising the VH and VL amino acid sequences of SEQ ID NOs: 137 and 138, respectively, and an antibody or an antigen-binding portion thereof comprising the VH and VL amino acid sequences of SEQ ID NOs: 147 and 148, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 69 and 70, respectively; and an anti-EGFR combination comprising an antibody comprising the HC and LC amino acid sequences of SEQ ID NOs: 139 and 140, respectively, and an antibody comprising the HC and LC amino acid sequences of SEQ ID NOs: 149 and 150, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 61-66, respectively; and an anti-HER2 antibody or an antigen-binding portion thereof comprising the H-CDR1-3 and L-CDR1-3 amino acid sequences of SEQ ID NOs: 161-166, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 67 and 68, respectively; and an anti-HER2 antibody or an antigen-binding portion thereof comprising the VH and VL amino acid sequences of SEQ ID NOs: 167 and 168, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 69 and 70, respectively; and an anti-HER2 antibody comprising the HC and LC amino acid sequences of SEQ ID NOs: 169 and 170, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 71-76, respectively; and an anti-HER2 antibody or an antigen-binding portion thereof comprising the H-CDR1-3 and L-CDR1-3 amino acid sequences of SEQ ID NOs: 161-166, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 77 and 78, respectively; and an anti-HER2 antibody or an antigen-binding portion thereof comprising the VH and VL amino acid sequences of SEQ ID NOs: 167 and 168, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 79 and 80, respectively; and an anti-HER2 antibody comprising the HC and LC amino acid sequences of SEQ ID NOs: 169 and 170, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; and an anti-PD-1 antibody or an antigen-binding portion thereof comprising the H-CDR1-3 and L-CDR1-3 amino acid sequences of SEQ ID NOs: 31-36, respectively;
an anti-NKG2A antibody or an antigen-binding portion thereof comprising the VH and VL amino acid sequences of SEQ ID NOs: 7 and 8, respectively; and an anti-PD-1 antibody or an antigen-binding portion thereof comprising the VH and VL amino acid sequences of SEQ ID NOs: 37 and 38, respectively; or
an anti-NKG2A antibody comprising the HC and LC amino acid sequences of SEQ ID NOs: 9 and 10, respectively; and an anti-PD-1 antibody comprising the HC and LC amino acid sequences of SEQ ID NOs: 39 and 40, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-HER2 antibody or an antigen-binding portion thereof comprising the H-CDR1-3 and L-CDR1-3 amino acid sequences of SEQ ID NOs: 151-156, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-HER2 antibody or an antigen-binding portion thereof comprising the VH and VL amino acid sequences of SEQ ID NOs: 157 and 158, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-HER2 antibody comprising the HC and LC amino acid sequences of SEQ ID NOs: 159 and 160, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 21-26, respectively; and an anti-HER2 antibody or an antigen-binding portion thereof comprising the H-CDR1-3 and L-CDR1-3 amino acid sequences of SEQ ID NOs: 151-156, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 27 and 28, respectively; and an anti-HER2 antibody or an antigen-binding portion thereof comprising the VH and VL amino acid sequences of SEQ ID NOs: 157 and 158, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 29 and 30, respectively; and an anti-HER2 antibody comprising the HC and LC amino acid sequences of SEQ ID NOs: 159 and 160, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-EGFR combination comprising an antibody or an antigen-binding portion thereof comprising the H-CDR1-3 and L-CDR1-3 amino acid sequences of SEQ ID NOs: 131-136, respectively, and an antibody or an antigen-binding portion thereof comprising the H-CDR1-3 and L-CDR1-3 amino acid sequences of SEQ ID NOs: 141-146, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-EGFR combination comprising an antibody or an antigen-binding portion thereof comprising the VH and VL amino acid sequences of SEQ ID NOs: 137 and 138, respectively, and an antibody or an antigen-binding portion thereof comprising the VH and VL amino acid sequences of SEQ ID NOs: 147 and 148, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-EGFR combination comprising an antibody comprising the HC and LC amino acid sequences of SEQ ID NOs: 139 and 140, respectively, and an antibody comprising the HC and LC amino acid sequences of SEQ ID NOs: 149 and 150, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 21-26, respectively; and an anti-EGFR combination comprising an antibody or an antigen-binding portion thereof comprising the H-CDR1-3 and L-CDR1-3 amino acid sequences of SEQ ID NOs: 131-136, respectively, and an antibody or an antigen-binding portion thereof comprising the H-CDR1-3 and L-CDR1-3 amino acid sequences of SEQ ID NOs: 141-146, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 27 and 28, respectively; and an anti-EGFR combination comprising an antibody or an antigen-binding portion thereof comprising the VH and VL amino acid sequences of SEQ ID NOs: 137 and 138, respectively, and an antibody or an antigen-binding portion thereof comprising the VH and VL amino acid sequences of SEQ ID NOs: 147 and 148, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 29 and 30, respectively; and an anti-EGFR combination comprising an antibody comprising the HC and LC amino acid sequences of SEQ ID NOs: 139 and 140, respectively, and an antibody comprising the HC and LC amino acid sequences of SEQ ID NOs: 149 and 150, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-EGFR antibody or an antigen-binding portion thereof comprising the H-CDR1-3 and L-CDR1-3 amino acid sequences of SEQ ID NOs: 111-116, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-EGFR antibody or an antigen-binding portion thereof comprising the VH and VL amino acid sequences of SEQ ID NOs: 117 and 118, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-EGFR antibody comprising the HC and LC amino acid sequences of SEQ ID NOs: 119 and 120, respectively.

In certain embodiments, the combination therapy or composition of the present disclosure comprises:
an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 21-26, respectively; and an anti-EGFR antibody or an antigen-binding portion thereof comprising the H-CDR1-3 and L-CDR1-3 amino acid sequences of SEQ ID NOs: 111-116, respectively;
an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 27 and 28, respectively; and an anti-EGFR antibody or an antigen-binding portion thereof comprising the VH and VL amino acid sequences of SEQ ID NOs: 117 and 118, respectively; or
an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 29 and 30, respectively; and an anti-EGFR antibody comprising the HC and LC amino acid sequences of SEQ ID NOs: 119 and 120, respectively.

### Multi-Specific Binding Molecules

In a further aspect, the present disclosure provides a multi-specific binding molecule having the binding specificity (e.g., comprising the antigen-binding portions, such as antigen-binding domains comprising the six CDRs) of an anti-NKG2A antibody or antigen-binding portion thereof in combination with the binding specificity of (1) an anti-PD-1 antibody, (2) an anti-PD-L1 antibody, (3) an anti-EGFR antibody, (4) an anti-HER2 antibody, or (5) any combination thereof. In certain embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof, and
b) an anti-PD-1 or anti-PD-L1 antibody or an antigen-binding portion thereof.
   In certain embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof,
b) an anti-PD-1 or anti-PD-L1 antibody or an antigen-binding portion thereof, and
c) an anti-EGFR or anti-HER2 antibody or an antigen-binding portion thereof.
   The anti-NKG2A antibody, anti-PD-1 antibody, anti-PD-L1 antibody, anti-EGFR antibody, and/or anti-HER2 antibody may be an antibody to said target as described herein.

In some embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof as described herein (e.g., mAb1 or mAb2), and
b) an anti-PD-1 antibody or an antigen-binding portion thereof as described herein (e.g., nivolumab, pembrolizumab, cemiplimab, dostarlimab, mAb3, or retifanlimab).

In some embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof as described herein (e.g., mAb1 or mAb2), and
b) an anti-PD-L1 antibody or an antigen-binding portion thereof as described herein (e.g., atezolizumab, avelumab, or durvalumab).

In some embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof as described herein (e.g., mAb1 or mAb2),
b) an anti-PD-1 antibody or an antigen-binding portion thereof as described herein (e.g., nivolumab, pembrolizumab, cemiplimab, dostarlimab, mAb3, or retifanlimab), and
c) an anti-EGFR antibody or an antigen-binding portion thereof as described herein (e.g., cetuximab, panitumumab, futuximab, modotuximab, or futuximab+modotuximab).

In some embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof as described herein (e.g., mAb1 or mAb2),
b) an anti-PD-L1 antibody or an antigen-binding portion thereof as described herein (e.g., atezolizumab, avelumab, or durvalumab), and
c) an anti-EGFR antibody or an antigen-binding portion thereof as described herein (e.g., cetuximab, panitumumab, futuximab, modotuximab, or futuximab+modotuximab).

In some embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof as described herein (e.g., mAb1 or mAb2),
b) an anti-PD-1 antibody or an antigen-binding portion thereof as described herein (e.g., nivolumab, pembrolizumab, cemiplimab, dostarlimab, mAb3, or retifanlimab), and
c) an anti-HER2 antibody or an antigen-binding portion thereof as described herein (e.g., trastuzumab, margetuximab, trastuzumab dexrutecan, or trastuzumab emtansine).

In some embodiments, the multi-specific binding molecule has the binding specificity (e.g., comprises the antigen-binding portions, such as antigen-binding domains comprising the six CDRs) of:
a) an anti-NKG2A antibody or an antigen-binding portion thereof as described herein (e.g., mAb1 or mAb2),
b) an anti-PD-L1 antibody or an antigen-binding portion thereof as described herein (e.g., atezolizumab, avelumab, or durvalumab), and
c) an anti-HER2 antibody or an antigen-binding portion thereof as described herein (e.g., trastuzumab, margetuximab, trastuzumab dexrutecan, or trastuzumab emtansine).

In some embodiments, the multi-specific binding molecule has the binding specificity (e.g., comprises the antigen-binding portions, such as antigen-binding domains comprising the six CDRs or the VH and VL) of:
(1) mAb1 and (2) mAb3;
(1) mAb1 and pembrolizumab;
(1) mAb1 and (2) cetuximab;
(1) mAb1 and (2) futuximab+modotuximab;
(1) mAb1 and (2) avelumab;
(1) mAb1, (2) mAb3, and (3) futuximab+modotuximab;
(1) mAb1, (2) mAb3, and (3) margetuximab;
(1) mAb1, (2) retifanlimab, and (3) margetuximab;
(1) mAb1, (2) pembrolizumab, and (3) trastuzumab;
(1) mAb1, (2) nivolumab, and (3) trastuzumab;
(1) mAb1, (2) pembrolizumab, and (3) futuximab+modotuximab;
(1) mAb1, (2) nivolumab, and (3) futuximab+modotuximab;
(1) mAb1, (2) nivolumab, and (3) cetuximab or
(1) mAb1, (2) pembrolizumab, and (3) cetuximab.

Multi-specific binding molecules are known in the art, and examples of different types of multi-specific binding molecules are given elsewhere herein. Such multi-specific (e.g., bi-specific or trispecific) binding molecules are encompassed by the therapies of the present disclosure.

### Nucleic Acid Molecules and Vectors

Also described are nucleic acid molecules and sequences antibodies or antigen-binding portions thereof described herein. In some embodiments, different nucleic acid molecules encode the heavy chain and light chain amino acid sequences of the antibodies or antigen-binding portions. In other embodiments, the same nucleic acid molecule encodes the heavy chain and light chain amino acid sequences of the antibodies or antigen-binding portions.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single- and double-stranded forms.

In some embodiments, the present disclosure provides a nucleic acid molecule comprising a nucleotide sequence that encodes the heavy chain or an antigen-binding portion thereof, or a nucleotide sequence that encodes the light chain or an antigen-binding portion thereof, or both, of an antibody or antigen-binding portion thereof described herein.

The present disclosure also provides nucleotide sequences that are at least 70%, 75%, 80%, 85%, 90%, 95%, 97%, 98% or 99% identical to a nucleotide sequence encoding an amino acid sequence selected from the group consisting of SEQ ID NOs: 7-10, 17-20, 27-30, 37-40, 47-50, 57-60, 67-70, 77-80, 87-90, 97-100, 107-110, 117-120, 127-130, 137-140, 147-150, 157-160, or 167-170. The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides.

There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wisconsin. FASTA, which includes, e.g., the programs FASTA2 and FASTA3, provides alignments and percent sequence identity of the regions of the best overlap between the query and search sequences (see, e.g., Pearson, Methods Enzymol. (1990) 183:63-98; Pearson, Methods Mol. Biol. (2000) 132:185-219; Pearson, Methods Enzymol. (1996) 266:227-58; and Pearson, J. Mol. Biol. (1998) 276:71-84; incorporated herein by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, incorporated herein by reference.

In any of the above embodiments, the nucleic acid molecules may be isolated. Nucleic acid molecules referred to herein as “isolated” or “purified” are nucleic acids which (1) have been separated away from the nucleic acids of the genomic DNA or cellular RNA of their source of origin; and/or (2) do not occur in nature.

In a further aspect, the present disclosure provides a vector suitable for expressing one or both of the chains of an antibody or antigen-binding portion thereof as described herein. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. Moreover, certain vectors are capable of directing the expression of genes to which they are operatively linked. Such vectors are referred to herein as “recombinant expression vectors” (or simply, “expression vectors”).

The present disclosure provides vectors comprising nucleic acid molecules that encode the heavy chain, the light chain, or both the heavy and light chains of an antibody as described herein or an antigen-binding portion thereof. In certain embodiments, a vector of the present disclosure comprises a nucleic acid molecule described herein. The present disclosure further provides vectors comprising nucleic acid molecules encoding fusion proteins, modified antibodies, antibody fragments, and probes thereof. The vector may further comprise an expression control sequence.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

In some embodiments, a nucleic acid molecule as described herein comprises a nucleotide sequence encoding a VH domain from an antibody or antigen-binding portion as described herein joined in-frame to a nucleotide sequence encoding a heavy chain constant region from any source. Similarly, a nucleic acid molecule as described herein can comprise a nucleotide sequence encoding a VL domain from an antibody or antigen-binding portion as described herein joined in-frame to a nucleotide sequence encoding a light chain constant region from any source.

In a further aspect of the present disclosure, nucleic acid molecules encoding the VH and/or VL may be “converted” to full-length antibody genes. In some embodiments, nucleic acid molecules encoding the VH or VL domains are converted to full-length antibody genes by insertion into an expression vector already encoding heavy chain constant (CH) or light chain constant (CL) regions, respectively, such that the VH segment is operatively linked to the CH segment(s) within the vector, and/or the VL segment is operatively linked to the CL segment within the vector. In another aspect, nucleic acid molecules encoding the VH and/or VL domains are converted into full-length antibody genes by linking, e.g., ligating, a nucleic acid molecule encoding a VH and/or VL domain to a nucleic acid molecule encoding a CH and/or CL region using standard molecular biological techniques. Nucleic acid molecules encoding the full-length heavy and/or light chains may then be expressed from a cell into which they have been introduced and the antibody isolated.

In some embodiments, the framework region(s) are mutated so that the resulting framework region(s) have the amino acid sequence of the corresponding germline gene. A mutation may be made in a framework region or constant region, e.g., to increase the half-life of the antibody. See, e.g., PCT Publication WO 00/09560. A mutation in a framework region or constant region also can be made to alter the immunogenicity of the antibody, and/or to provide a site for covalent or non-covalent binding to another molecule. According to the present disclosure, an antibody may have mutations in any one or more of the CDRs or framework regions of the variable domain or in the constant region.

### Host Cells and Methods of Antibody and Antibody Composition Production

Also described are methods for producing the combination therapies (e.g., compositions) of the present disclosure. One embodiment relates to a method for producing antibodies as described herein, comprising providing recombinant host cells capable of expressing the antibodies, culturing said host cells under conditions suitable for expression of the antibodies, and isolating the resulting antibodies.

Antibodies produced by such expression in such recombinant host cells are referred to herein as “recombinant antibodies.” Also described are progeny cells of such host cells, and antibodies produced by same.

The term “recombinant host cell” (or simply “host cell”), as used herein, means a cell into which a recombinant expression vector has been introduced. By definition, a recombinant host cell does not occur in nature. The present disclosure provides host cells that may comprise, e.g., a vector as described herein. The present disclosure also provides host cells that comprise, e.g., a nucleotide sequence encoding the heavy chain or an antigen-binding portion thereof, a nucleotide sequence encoding the light chain or an antigen-binding portion thereof, or both, of an antibody or antigen-binding portion thereof described herein. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding antibodies or antigen-binding portions thereof described herein and vectors comprising these nucleic acid molecules can be used for transfection of a suitable mammalian, plant, bacterial or yeast host cell.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

It is likely that antibodies expressed by different cell lines or in transgenic animals will have different glycosylation patterns from each other. However, all antibodies encoded by the nucleic acid molecules provided herein, or comprising the amino acid sequences provided herein are part of the present disclosure, regardless of the glycosylation state of the antibodies, and more generally, regardless of the presence or absence of post-translational modification(s).

In some embodiments, the present disclosure relates to a method for producing an antibody composition comprising an anti-NKG2A antibody or an antigen-binding portion thereof and an anti-PD-1 or anti-PD-L1 antibody or an antigen-binding portion thereof, the method comprising:
providing first and second host cells, wherein the first host cell is capable of expressing an anti-NKG2A antibody or an antigen-binding portion thereof as described herein and the second host cell is capable of expressing an anti-PD-1 or anti-PD-L1 antibody or antigen-binding portion thereof as described herein,
cultivating the first and second and host cells under conditions suitable for expression of the anti-NKG2A antibody or antigen-binding portion thereof and the anti-PD-1 or anti-PD-L1 antibody or antigen-binding portion thereof, and
isolating the resulting antibodies.

In some embodiments, the present disclosure relates to a method for producing an antibody composition comprising an anti-NKG2A antibody or an antigen-binding portion thereof, an anti-PD-1 or anti-PD-L1 antibody or an antigen-binding portion thereof, and an anti-EGFR or anti-HER2 antibody or an antigen-binding portion thereof, the method comprising:
providing first, second, and third host cells, wherein the first host cell is capable of expressing an anti-NKG2A antibody or antigen-binding portion thereof as described herein, the second host cell is capable of expressing an anti-PD-1 or anti-PD-L1 antibody or antigen-binding portion thereof as described herein, and the third host cell is capable of expressing an anti-EGFR or anti-HER2 antibody or antigen-binding portion thereof as described herein,
cultivating the first, second, and third host cells under conditions suitable for expression of the anti-NKG2A antibody or antigen-binding portion thereof, the anti-PD-1 or anti-PD-L1 antibody or antigen-binding portion thereof, and the anti-EGFR antibody or antigen-binding portion thereof, and
isolating the resulting antibodies.

The present disclosure also provide host cells comprising:
a nucleotide sequence that encodes the heavy chain or an antigen-binding portion thereof, a nucleotide sequence that encodes the light chain or an antigen-binding portion thereof, or both, of an anti-NKG2A antibody or antigen-binding portion as described herein, and a nucleotide sequence that encodes the heavy chain or an antigen-binding portion thereof, a nucleotide sequence that encodes the light chain or an antigen-binding portion thereof, or both, of an anti-PD-1 or PD-L1 antibody as described herein; or
a nucleotide sequence that encodes the heavy chain or an antigen-binding portion thereof, a nucleotide sequence that encodes the light chain or an antigen-binding portion thereof, or both, of an anti-NKG2A antibody or antigen-binding portion as described herein; a nucleotide sequence that encodes the heavy chain or an antigen-binding portion thereof, a nucleotide sequence that encodes the light chain or an antigen-binding portion thereof, or both, of an anti-PD-1 or PD-L1 antibody as described herein; and a nucleotide sequence that encodes the heavy chain or an antigen-binding portion thereof, a nucleotide sequence that encodes the light chain or an antigen-binding portion thereof, or both, of an anti-EGFR or anti-HER2 antibody as described herein.

### Pharmaceutical Compositions

Another aspect of the present disclosure is a pharmaceutical composition comprising as active ingredients (e.g., as the sole active ingredients):
an anti-NKG2A antibody or antigen-binding portion thereof as described herein;
an anti-NKG2A antibody or antigen-binding portion thereof as described herein and an anti-PD-1 or anti-PD-L1 antibody or antigen-binding portion thereof as described herein;
an anti-NKG2A antibody or antigen-binding portion thereof as described herein and an anti-EGFR or anti-HER2 antibody or antigen-binding portion thereof as described herein; or
an anti-NKG2A antibody or antigen-binding portion thereof as described herein, an anti-PD-1 or PD-L1 antibody or antigen-binding portion thereof as described herein, and an anti-EGFR or anti-HER2 antibody or antigen-binding portion thereof as described herein.
   In some aspects, the pharmaceutical composition comprises a multi-specific binding molecule (e.g., a multi-specific binding molecule that has the binding specificity of an anti-NKG2A antibody as described herein and an anti-PD-1 or anti-PD-L1 antibody as described herein; or an anti-NKG2A antibody, an anti-PD-1 or anti-PD-L1 antibody, and an anti-EGFR or anti-HER2 antibody as described herein).
   Another aspect of the present disclosure is a pharmaceutical composition comprising as an active ingredient (or as the sole active ingredient) a monotherapy or combination therapy of the present disclosure. The pharmaceutical composition may additionally comprise a pharmaceutically acceptable excipient. In some embodiments, the pharmaceutical compositions are intended for amelioration, prevention, and/or treatment of cancer, e.g., a cancer described herein. In certain embodiments, the cancer is in a tissue such as skin, lung, intestine, colon, ovary, brain, prostate, kidney, soft tissues, the hematopoietic system, head and neck, liver, bone, bladder, breast, stomach, uterus, cervix, and pancreas.

Pharmaceutical compositions of the present disclosure will comprise one or more antibodies, antigen-binding portions, antibody compositions, or multi-specific binding molecules as described herein. In some embodiments, the composition comprises two antibodies described herein or antigen-binding portions thereof. In some embodiments, the composition comprises three antibodies as described herein or antigen-binding portions thereof. In some embodiments, the composition comprises a monotherapy or combination therapy described herein.

In some embodiments, the pharmaceutical composition may comprise a monotherapy or combination therapy of the present disclosure, and one or more additional agents selected from, e.g., an immunostimulatory agent, a vaccine, a chemotherapeutic agent, an anti-neoplastic agent, an anti-angiogenic agent, and a tyrosine kinase inhibitor.

In some embodiments, the pharmaceutical composition is intended for amelioration, prevention, and/or treatment of a disorder, disease, or condition that improves, or slows down in its progression, by modulation of NKG2A, PD-1, PD-L1, EGFR, HER2, or any combination thereof. In some embodiments, the pharmaceutical composition is intended for amelioration, prevention, and/or treatment of cancer. In some embodiments, the pharmaceutical composition is intended for activation of the immune system.

Generally, the therapies and compositions of the present disclosure are suitable to be administered as one or more formulations in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present disclosure and methods for their preparation will be readily apparent to those skilled in the art. Such compositions and methods for their preparation may be found, for example, in Remington's Pharmaceutical Sciences, 19th Edition (Mack Publishing Company, 1995).

Pharmaceutical compositions are preferably manufactured under GMP (good manufacturing practices) conditions.

A pharmaceutical composition of the present disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation.

### Therapeutic Uses of Antibodies and Compositions of the Present Disclosure

In some embodiments, the therapies and compositions of the present disclosure are used to enhance or activate the immune system in a patient (e.g., a mammal such as a human) in need thereof. In certain embodiments, the patient is immune-suppressed. In certain embodiments, a physician can boost the anti-cancer activity of a patient's own immune system by administering a therapy or composition as described herein. For example, a physician can boost anti-tumor activity in a patient by administering a therapy or composition of the present disclosure, alone or in combination with other therapeutic agents (sequentially or concurrently).

In certain embodiments, the therapies or compositions of the present disclosure are for use in the treatment of cancer. The cancer may be in one or more tissues such as skin, lung, intestine, colon, ovary, brain, prostate, kidney, soft tissues, the hematopoietic system, head and neck, liver, bone, bladder, breast, stomach, uterus, cervix, and pancreas.

In some embodiments, cancers treated by the therapies and compositions of the present disclosure may include, e.g., melanoma (e.g., advanced or metastatic melanoma), skin basal cell cancer, glioblastoma, glioma, gliosarcoma, astrocytoma, meningioma, neuroblastoma, adrenocortical cancer, head and neck squamous cell cancer, oral cancer, salivary gland cancer, nasopharyngeal cancer, breast cancer, lung cancer (e.g., non-small cell lung cancer (NSCLC), small cell lung cancer, and squamous cell lung cancer), esophageal cancer, gastroesophageal junction cancer, gastric cancer, gastrointestinal cancer, primary peritoneal cancer, liver cancer, hepatocellular carcinoma, biliary tract cancer, colon cancer, rectal cancer, colorectal carcinoma, ovarian cancer, fallopian tube cancer, bladder cancer, upper urinary tract cancer, urothelial cancer, renal cell carcinoma, kidney cancer, genitourinary cancer, cervical cancer, prostate cancer, fibrosarcoma, liposarcoma, rhabdomyosarcoma, osteosarcoma, histiocytoma, pancreatic cancer, endometrial cancer, cancer of the appendix, advanced Merkel cell cancer, multiple myeloma, sarcomas, choriocarcinoma, erythroleukemia, acute lymphoblastic leukemia, acute monocytic leukemia, acute promyelocytic leukemia, acute myeloid leukemia, chronic myeloid leukemia, chronic lymphocytic leukemia, acute lymphoblastic leukemia, mast cell leukemia, small lymphocytic lymphoma, Burkitt's lymphoma, Hodgkin's lymphoma, non-Hodgkin's lymphoma, diffuse large B cell lymphoma, follicular lymphoma, monocytic lymphoma, HTLV-associated T cell leukemia/lymphoma, mesothelioma, and solid tumors. The cancer may be, e.g., at an early, intermediate, late, locally advanced, advanced, or metastatic stage, may be relapsed, and may be refractory to and/or intolerant of other therapeutics (e.g., other therapeutics directed to one or more targets of the therapy or composition, checkpoint inhibitors, or standard of care for the cancer) or there may be no standard therapy available. In some embodiments, the cancer may not be amenable to surgical intervention due to either medical contraindications or non-resectability of the tumor.

In some embodiments, conditions treated by the therapies and compositions of the present disclosure may include, e.g., gastric and colorectal cancer. In some embodiments, the gastric or colorectal cancer is metastatic, locally advanced, or unresectable.

In some embodiments, the gastric cancer is (1) unresectable, (2) locally advanced or metastatic, (3) HER2+, or (4) any combination (e.g., all) of (1)-(3). Additionally or alternatively, the patient with the gastric cancer may have received treatment with first line standard therapy (e.g., cytotoxic chemotherapy, trastuzumab, and/or pembrolizumab). In certain embodiments, a therapy or composition of the present disclosure (e.g., targeting NKG2A, PD-1, and HER2, such as mAb1+mAb3+margetuximab) may be used to treat locally advanced unresectable or metastatic HER2⁺ gastric cancer, e.g., where first-line standard of care therapy such as cytotoxic chemotherapy, trastuzumab, and/or pembrolizumab has failed.

In some embodiments, a therapy or composition of the present disclosure (e.g., targeting NKG2A, PD-1, and HER2, such as mAb1+ pembrolizumab+trastuzumab) may be used to treat locally advanced unresectable or metastatic HER2⁺ gastroesophageal junction (GEJ) and gastric adenocarcinoma (GA)., e.g., where first-line standard of care therapy such as cytotoxic chemotherapy, trastuzumab, and/or pembrolizumab has failed.

In some embodiments, the colorectal cancer is (1) metastatic, (2) not amenable to surgical intervention due to either medical contraindications or non-resectability of the tumor, (3) with microsatellite instability status as low per institutional guidelines or guidelines from the College of American Pathologists, e.g. MSI-H cancer, (4) any combination (e.g., all) of (1)-(3). Additionally or alternatively, the patient with the colorectal cancer may be (i) without RAS (KRAS and NRAS) mutations in any of the following codons: codons 12 and 13 in exon 2, codons 59 and 61 in exon 3, and codons 117 and 146 in exon 4; and/or (ii) without BRAF V600E mutation. In certain embodiments, a therapy or composition of the present disclosure (e.g., targeting NKG2A, PD-1, and EGFR, such as mAb1+mAb3+futuximab+modotuximab) may be used to treat metastatic colorectal cancer. In certain embodiments, a therapy or composition of mAb1 and pembrolizumab may be used to treat colorectal cancer and specifically MSI-H/dMMR locally advanced unresectable or metastatic colorectal cancer (mCRC).

In some embodiments, the therapies or compositions of the present disclosure may be used to treat a patient population as described in Example 10.

In some embodiments, a therapy or composition described herein may inhibit tumor growth and/or induce tumor growth regression in vivo. In some embodiments, a therapy or composition described herein may slow down or reverse metastasis in a cancer patient. In some embodiments, a therapy or composition described herein may prolong survival of a cancer patient. Any combination of the above properties is also contemplated.

In some embodiments, the therapies or compositions of the present disclosure may be used in the treatment of an immune disorder.

In some embodiments, the therapies or compositions of the present disclosure may be used to treat a patient who is, or is at risk of being, immunocompromised (e.g., due to chemotherapeutic or radiation therapy). In some embodiments, the therapies or compositions may be used to expand stem cells in a patient after stem cell transplantation.

In some embodiments, the therapy or composition is for use in treating viral and/or parasitic infections, e.g., where the pathogens inhibit the host immune response. The pathogen may be, e.g., HIV, hepatitis (A, B, or C), human papilloma virus (HPV), lymphocytic choriomeningitis virus (LCMV), adenovirus, flavivirus, echovirus, rhinovirus, coxsackie virus, coronavirus, respiratory syncytial virus, mumps virus, rotavirus, measles virus, rubella virus, parvovirus, vaccinia virus, human T-cell lymphotrophic virus (HTLV), human cytomegalovirus (HCMV), dengue virus, molluscum virus, poliovirus, rabies virus, John Cunningham (JC) virus, arboviral encephalitis virus, simian immunodeficiency virus (SIV), influenza, herpes, Giardia, malaria, Leishmania, Staphylococcus aureus, or Pseudomonas aeruginosa.

“Treat,” “treating,” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Therapeutically effective amount” refers to the amount of the therapeutic agent being administered that will relieve to some extent one or more of the symptoms of the disorder being treated. A therapeutically effective amount of an anti-cancer therapeutic may, for example, result in delayed tumor growth, tumor shrinkage, increased survival, elimination of cancer cells, slowed or decreased disease progression, reversal of metastasis, or other clinical endpoints desired by healthcare professionals. In some embodiments, a therapeutically effective amount of a therapy or composition of the present disclosure results in an improved objective response rate, improved clinical benefit rate, improved duration of response, increased progression-free survival, and increased overall survival, e.g., in comparison to untreated patients.

In some embodiments, a therapy as described herein is administered in a single composition. In other embodiments, the therapy (e.g., a combination therapy) is administered in more than one composition. For example, a combination therapy comprising an anti-NKG2A antibody, an anti-PD-1 or anti-PD-L1 antibody, and an anti-EGFR or anti-HER2 antibody may involve administration of a single composition comprising all three antibodies, a composition comprising two of the antibodies and a composition comprising one of the antibodies, or a separate composition for each antibody. In a case where there is more than one composition, the compositions can be administered simultaneously, sequentially, separately, or any combination thereof.

The therapies or compositions of the present disclosure may be administered without additional therapeutic treatments, i.e., as a stand-alone therapy (monotherapy). Alternatively, treatment with the therapy or combination may include at least one additional therapeutic treatment, e.g., another immunostimulatory agent, an anti-cancer agent (e.g., a chemotherapeutic agent, an anti-neoplastic agent, an anti-angiogenic agent, or a tyrosine kinase inhibitor), or a vaccine (e.g., a tumor vaccine).

In some embodiments, the therapy or composition may be co-administered or formulated with another medication/drug for the treatment of cancer. The additional therapeutic treatment may comprise, e.g., an immunostimulatory agent, a vaccine, a chemotherapeutic agent, an anti-neoplastic agent, an anti-angiogenic agent, a tyrosine kinase inhibitor, and/or radiation therapy. In some embodiments, the additional therapeutic treatment may comprise a different anti-cancer antibody.

Pharmaceutical articles comprising a therapy or composition as described herein and at least one other agent (e.g., a chemotherapeutic, anti-neoplastic, or anti-angiogenic agent) may be used as a combination treatment for simultaneous, separate or successive administration in cancer therapy. The other agent may by any agent suitable for treatment of the particular cancer in question, for example, an agent selected from the group consisting of alkylating agents, e.g., platinum derivatives such as cisplatin, carboplatin and/or oxaliplatin; plant alkoids, e.g., paclitaxel, docetaxel and/or irinotecan; antitumor antibiotics, e.g., doxorubicin (adriamycin), daunorubicin, epirubicin, idarubicin mitoxantrone, dactinomycin, bleomycin, actinomycin, luteomycin, and/or mitomycin; topoisomerase inhibitors such as topotecan; antimetabolites, e.g., fluorouracil and/or other fluoropyrimidines; FOLFOX; osimertinib; cyclophosphamide; anthracycline; dacarbazine; gemcitabine; or any combination thereof. In some embodiments, the therapy or composition described herein reestablishes responsiveness to the other agent.

A therapy or composition of the present disclosure may also be used in combination with other anti-cancer therapies such as vaccines, cytokines, enzyme inhibitors, immunostimulatory compounds, and T cell therapies. In the case of a vaccine, it may be, e.g., a protein, peptide, or DNA vaccine containing one or more antigens which are relevant for the cancer being treated, or a vaccine comprising dendritic cells along with an antigen. Suitable cytokines include, for example, IL-2, IFN-gamma and GM-CSF. An example of a type of enzyme inhibitor that has anti-cancer activity is an indoleamine-2,3-dioxygenase (IDO) inhibitor, for example, 1-methyl-D-tryptophan (1-D-MT). Also contemplated is adoptive T cell therapy, which refers to various immunotherapy techniques that involve expanding or engineering patients' own T cells to recognize and attack their tumors.

It is also contemplated that a therapy or composition of the present disclosure may be used in adjunctive therapy in connection with tyrosine kinase inhibitors. These are synthetic, mainly quinazoline-derived, low molecular weight molecules that interact with the intracellular tyrosine kinase domain of receptors and inhibit ligand-induced receptor phosphorylation, e.g., by competing for the intracellular Mg-ATP binding site.

In some embodiments, the therapy or composition may be used in combination with a medication/drug that mediates immune system activation, including, but not limited to, an agent that modulates the expression or activity of A2AR, A1AR, A2BR, A3AR, ADA, ALP, AXL, BTLA, B7-H3, B7-H4, CTLA-4, CD116, CD123, CD27, CD28, CD39, CD40, CD47, CD55, CD73, CD122, CD137, CD160, CGEN-15049, CHK1, CHK2, CTLA-3, CEACAM (e.g., CEACAM-1 and/orCEACAM-5), EGFR, FLT3, HER2, NKG2AL, GAL9, GITR, HVEM, LAG-3, LILRB1, LY108, LAIR1, MET, NKG2A, ICOS, IDO, IL2R, IL4R, KIR, LAIR1, PAP, PD-1/PD-L1/PD-L2, OX40, STING, TIGIT, TIM-3, TGFR-beta, TLR1, TLR2, TLR3, TLR4, TLR5, TLR6, TLR7, TLR8, TLR9 and TLR10, TNFR2, VEGFR, VEGF, VISTA, LILRB2, CMTM6 and/or 2B4. In certain embodiments, the agent is a small molecule inhibitor. In certain embodiments, the agent is an antibody or an antigen-binding fragment thereof that binds to one of the above molecules. It is also contemplated that a therapy or composition of the present disclosure may be used in combination with a cytokine (e.g., IL-1, IL-2, IL-12, IL-15 or IL-21), an EGFR inhibitor, a VEGF inhibitor, etc.

As used herein, the terms “co-administration,” “co-administered” and “in combination with,” referring to the therapies and compositions of the present disclosure with one or more other therapeutic agents, is intended to mean, and does refer to and include the following:
a) simultaneous administration of such therapy/composition of the present disclosure and therapeutic agent(s) to a patient in need of treatment, when such components are formulated together into a single dosage form which releases said components at substantially the same time to said patient,
b) substantially simultaneous administration of such therapy/composition of the present disclosure and therapeutic agent(s) to a patient in need of treatment, when such components are formulated apart from each other into separate dosage forms which are taken at substantially the same time by said patient, whereupon said components are released at substantially the same time to said patient,
c) sequential administration of such therapy/composition of the present disclosure and therapeutic agent(s) to a patient in need of treatment, when such components are formulated apart from each other into separate dosage forms which are taken at consecutive times by said patient with a significant time interval between each administration, whereupon said components are released at substantially different times to said patient; and
d) sequential administration of such therapy/composition of the present disclosure and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

It is understood that the therapies and compositions of the present disclosure may be used in a method of treatment as described herein, may be for use in a treatment as described herein, and/or may be for use in the manufacture of a medicament for a treatment as described herein.

### Dose and Route of Administration

The therapies and compositions of the present disclosure may be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the antibodies are being administered as a stand-alone treatment or in combination with one or more additional anti-cancer treatments.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated, each unit containing a predetermined quantity of active compound calculated to produce the desired therapeutic effect in association with the required pharmaceutical carrier. The specification for the dosage unit forms of the present disclosure are generally dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of the present disclosure may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining of appropriate dosages and regimens is well-known in the relevant art and would be understood to be encompassed by the skilled artisan once provided the teachings disclosed herein.

An effective amount for tumor therapy may be measured by its ability to stabilize disease progression and/or ameliorate symptoms in a patient, and preferably to reverse disease progression, e.g., by reducing tumor size. The ability of a therapy or composition of the present disclosure to inhibit cancer may be evaluated by in vitro assays, e.g., as described in the examples, as well as in suitable animal models that are predictive of the efficacy in human tumors. Suitable dosage regimens will be selected in order to provide an optimum therapeutic response in each particular situation, for example, administered as a single bolus or as a continuous infusion, and with possible adjustment of the dosage as indicated by the exigencies of each case.

The therapies and compositions of the present disclosure may be administered by any method for administering peptides, proteins or antibodies accepted in the art, and are typically suitable for parenteral administration. As used herein, “parenteral administration” includes any route of administration characterized by physical breaching of a tissue of a subject and administration through the breach in the tissue, thus generally resulting in the direct administration into the blood stream, into muscle, or into an internal organ. Parenteral administration thus includes, but is not limited to, administration by injection, by application through a surgical incision, by application through a tissue-penetrating non-surgical wound, and the like. In particular, parenteral administration is contemplated to include, but is not limited to, subcutaneous, intraperitoneal, intramuscular, intrasternal, intracisternal, intravenous, intraarterial, intrathecal, intraurethral, intracranial, intratumoral, and intrasynovial injection or infusions. Particular embodiments include the intravenous and the subcutaneous routes. In some embodiments, the administration is IV injection, e.g., IV infusion.

In some embodiments, the therapies and compositions of the present disclosure may be administered according to an exemplary dosing regimen described in Example 10, e.g., in relation to Parts 1a, 1b, 2a, and 2b of the described clinical study.

For example, in certain embodiments, the anti-NKG2A antibody or antigen-binding portion may be administered at a dose of 8, 20, 100, 300, 750, or 1500 mg, or at a dose of 8-20, 20-100, 100-300, 300-750, or 750-1500 mg (e.g., as a monotherapy, or as part of a combination therapy, as described herein). In particular embodiments, the anti-NKG2A antibody or antigen-binding portion is administered every 1, 2, 3, 4, 5, or 6 weeks. Further, the anti-NKG2A antibody or antigen-binding portion may be administered in a cycle of 7, 14, 28, 42, 56, 70, or 84 days.

In certain embodiments, the anti-PD-1 or anti-PD-L1 antibody or antigen-binding portion thereof may be administered at a dose of 50, 100, 150, 200, 250, 300, 350, 400, 450, or 500 mg, or at a dose of 50-100, 100-150, 150-200, 200-250, 250-300, 300-350, 350-400, 400-450, or 450-500 mg (e.g., as part of a combination therapy as described herein). In particular embodiments, the anti-PD-1 or anti-PD-L1 antibody or antigen-binding portion is administered every 1, 2, 3, 4, 5, or 6 weeks, and may in certain embodiments be administered after 1, 2, 3, or 4 cycles of the anti-NKG2A antibody or antigen-binding portion thereof.

In certain embodiments, the anti-EGFR antibody or antigen-binding portion thereof (e.g., a combination of two anti-EGFR antibodies or antigen-binding portions thereof) may be administered at a dose of 1, 3, 6, 9, 12, 15, or 18 mg/kg (e.g., as part of a combination therapy as described herein). In some embodiments, the anti-EGFR or antigen-binding portion thereof may be administered at one of said doses as a loading dose and a different one of said doses as a maintenance dose, for example a loading dose of 9 mg/kg followed by a maintenance dose of 6 mg/kg. In particular embodiments, the anti-EGFR antibody or antigen-binding portion thereof is administered every 1, 2, 3, or 4 weeks.

In certain embodiments, the anti-HER2 antibody or antigen-binding portion thereof may be administered at a dose of 5, 10, 15, 20, 25, or 30 mg/kg (e.g., as part of a combination therapy as described herein). In particular embodiments, the anti-HER2 antibody or antigen-binding portion thereof is administered every 1, 2, 3, 4, 5, 6, 7, or 8 weeks.

### Articles of Manufacture and Kits

The present disclosure also provides articles of manufacture comprising an anti-NKG2A antibody or an antigen-binding portion thereof as described herein, and optionally an anti-PD-1 or anti-PD-L1 antibody or antigen-binding portion thereof as described herein and/or an anti-EGFR or anti-HER2 antibody or antigen-binding portion as described herein. For example, the article of manufacture may comprise the antibodies of any therapy described herein, and may be for use in any treatment method described herein. Also provided are methods for manufacturing said articles.

The present disclosure also provides kits comprising an anti-NKG2A antibody or an antigen-binding portion thereof as described herein, and optionally an anti-PD-1 or anti-PD-L1 antibody or antigen-binding portion thereof as described herein and/or an anti-EGFR or anti-HER2 antibody or antigen-binding portion as described herein. For example, the kit may comprise the antibodies of any therapy described herein, and may be for use in any treatment method described herein.

The present disclosure also provides articles of manufacture and kits comprising one or more containers (e.g., single-use or multi-use containers) containing a therapy or composition described herein, optionally an additional biologically active molecule (e.g., another therapeutic agent), and instructions for use. The antibodies or antigen-binding portions of the therapy or composition, and optional additional biologically active molecule, can be packaged separately or together in any combination in suitable packing such as a vial or ampule made from non-reactive glass or plastic. In certain embodiments, the vial or ampule holds a concentrated stock (e.g., 2×, 5×, 10× or more) of the antibody or antigen-binding portion and/or the biologically active molecule. In certain embodiments, the articles of manufacture and kits include a medical device for administering the therapy or composition and/or the additional biologically active molecule (e.g., a syringe and a needle); and/or an appropriate diluent (e.g., sterile water and normal saline).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, immunology, microbiology, genetics, analytical chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

In order that the present disclosure may be better understood, the following examples are set forth. These examples are for purposes of illustration only and are not to be construed as limiting the scope of the present disclosure in any manner.

### ABBREVIATIONS

Abbreviations used herein are shown below.
ADCC antibody-dependent cellular cytotoxicity
AE adverse event
ALT alanine aminotransferase
ANC absolute neutrophil count
AST aspartate aminotransferase
AUC area under the plasma concentration-time curve
BOIN Bayesian optimal interval
CBR clinical benefit rate
Cmax maximum plasma concentration
CNS central nervous system
CPI checkpoint inhibitor
CPS combined positive score
CR complete response
CRC colorectal cancer
CTCAE Common Terminology Criteria for Adverse Events
DLT dose-limiting toxicity
DOR duration of response
ECG electrocardiogram
ECOG Eastern Cooperative Oncology Group
EGFR epidermal growth factor receptor
HCC hepatocellular carcinoma
HER human epidermal growth factor receptor
HLA human leucocyte antigen
IHC immunohistochemistry
IMP investigational medicinal product
INR international normalized ratio
IRR infusion-related reaction
IS included set
ISH in situ hybridization
IV intravenous
LVEF left ventricle ejection fraction
MAD maximum administered dose
MSI microsatellite instability
MTD maximum tolerated dose
NCI national cancer institute
NE not evaluable
NK natural killer (cell)
NYHA New York Heart Association
ORR objective response rate
OS overall survival
PBMC peripheral blood mononuclear cells
PD progressive disease
PFS progression-free survival
PK pharmacokinetics
PR partial response
RECIST Response Evaluation Criteria in Solid Tumors
SD stable disease
SRC Safety Review Committee
ULN upper limit of normal
VPC visual predictive check
WHO World Health Organization
WT wild-type

### EXAMPLES

### Example 1: NK Cell-Mediated Killing Induced by mAb1 in Selected Cell Lines Expressing Endogenous HLA-E

This example describes the expression of endogenous HLA-E on the surface of tumor cell lines (HT-29, CCRF-CEM, A253, Detroit 562, CAL-120, FaDu) and the effect of mAb1 on NK cell-mediated killing of these tumor cell lines in vitro.

### Materials and Methods

The expression of endogenous HLA-E in six different cell lines (HT-29, CCRF-CEM, A253, Detroit 562, CAL-120, and FaDu) was investigated by flow cytometry. Isolated human primary NK cells from healthy individuals were co-cultured with six different calcein-labeled target cells expressing endogenous HLA-E (loaded with HLA-B*0701 peptide) in a 10:1 ratio and treated with a single concentration of mAb1 or isotype control (IgG1 LALA). Release of calcein was measured after 1.5 hours and % specific lysis was calculated.

### Results

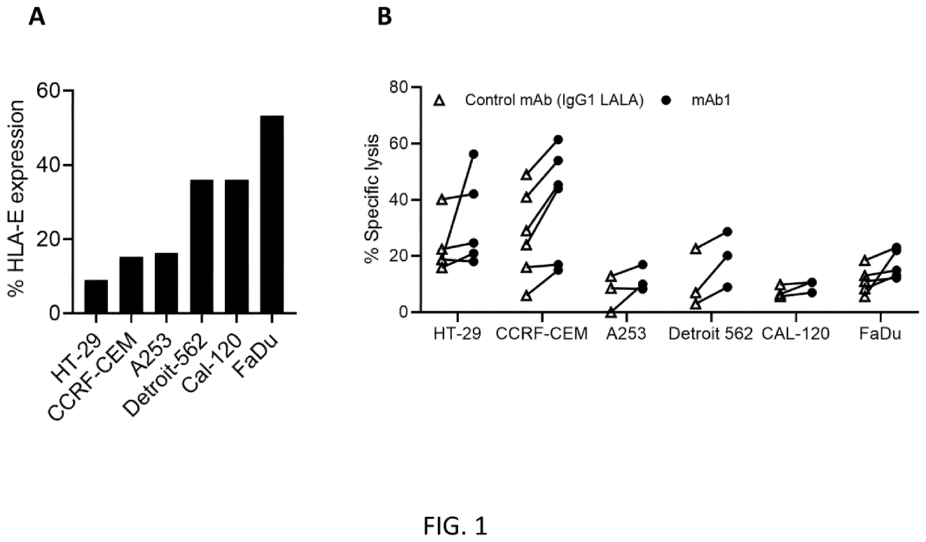

All six human tumor cell lines were shown express endogenous HLA-E on the surface (FIG. 1, Panel A). Treatment with mAb1 induced NK-mediated killing of these tumor cell lines compared to IgG1 LALA treatment (FIG. 1, Panel B).

### Example 2: Titration of mAb1 in Comparison to BMS Anti-NKG2A Antibody Analogues and Monalizumab Analogue

This example describes the activity of mAb1 in comparison to both monalizumab analogue and BMS anti-NKGA antibody analogues in a γδ T-cell cytotoxicity assay. Primary, expanded human γδ T cells derived from healthy individuals were co-cultured with HLA-E expressing target cells (K562-HLA-E) and treated with mAb1, a monalizumab analogue, analogues of BMS anti-NKG2A.9 (IgG1.3f), BMS anti-NKG2A.11 (IgG1.3f), or isotype control (IgG1-LALA).

### Materials and Methods

K562-HLA-E cells were HLA-B*0701 peptide-loaded overnight. The next day, human primary γδ T-cells derived and expanded from healthy individuals were isolated and incubated with a two-fold titration of the indicated antibodies starting from 50 μg/mL followed by addition of calcein-loaded K562-HLA-E target cells and incubation for 3 hours. The killing capacity of γδ T cells was measured by calcein release to the supernatant. Specific lysis was calculated by subtracting spontaneous lysis (calcein-loaded 562-HLA-E cells only) and normalizing to maximum lysis (Triton X-100 lysis of calcein-loaded K562-HLAE cells).

### Results

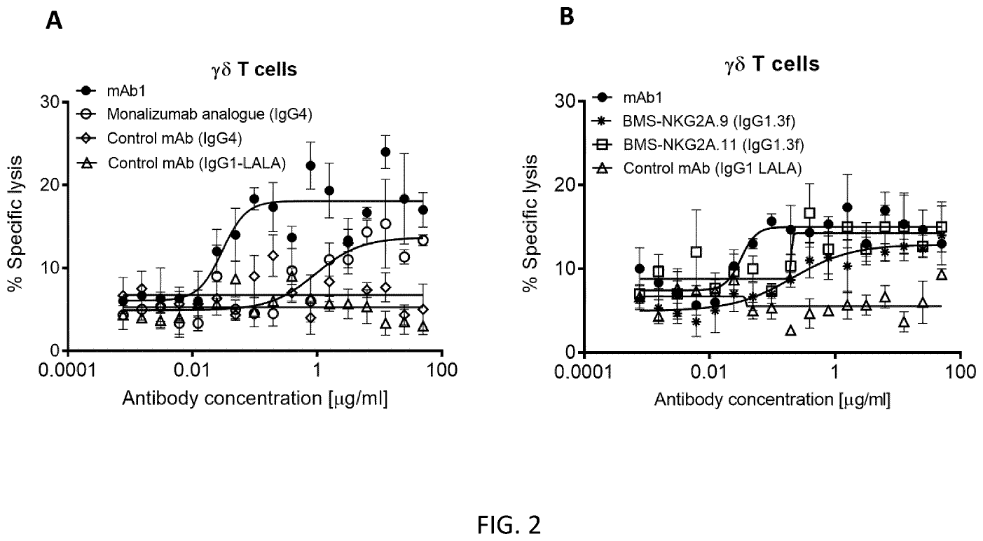

Head-to-head comparison with either monalizumab analogue (FIG. 2A) or BMS anti-NKGA antibody analogues (NKG2A.9 and NKG2A.11) (FIG. 2B) showed the superior functional activity of mAb1.

### Example 3: MAb1 Potentiates Cetuximab-Induced ADCC in FaDu Cells

This example describes the ability of mAb1 to enhance ADCC induced by EGFR targeting antibodies in a NK cell cytotoxicity assay using target cells that endogenously express both HLA-E and EGFR. A titration of cetuximab in combination with a fixed concentration of isotype control antibodies, mAb1, or monalizumab analogue was initially tested.

### Materials and Methods

FaDu cells were HLA-B*0701 peptide-loaded overnight. The next day, human primary NKG2A+γδ T cells derived and expanded from healthy individuals were cocultured with FaDu cells (1:10 E:T ratio) and incubated with a two-fold titration of cetuximab starting from 1 μg/mL in combination with the indicated antibodies at 25 μg/mL. After 90 minutes, the killing capacity of primary NK cells was measured by calcein release to the supernatant. Specific lysis was calculated by subtracting spontaneous lysis (calcein-loaded 562-HLA-E cells only) and normalizing to maximum lysis (Triton X-100 lysis of calcein-loaded K562-HLA-E cells).

### Results

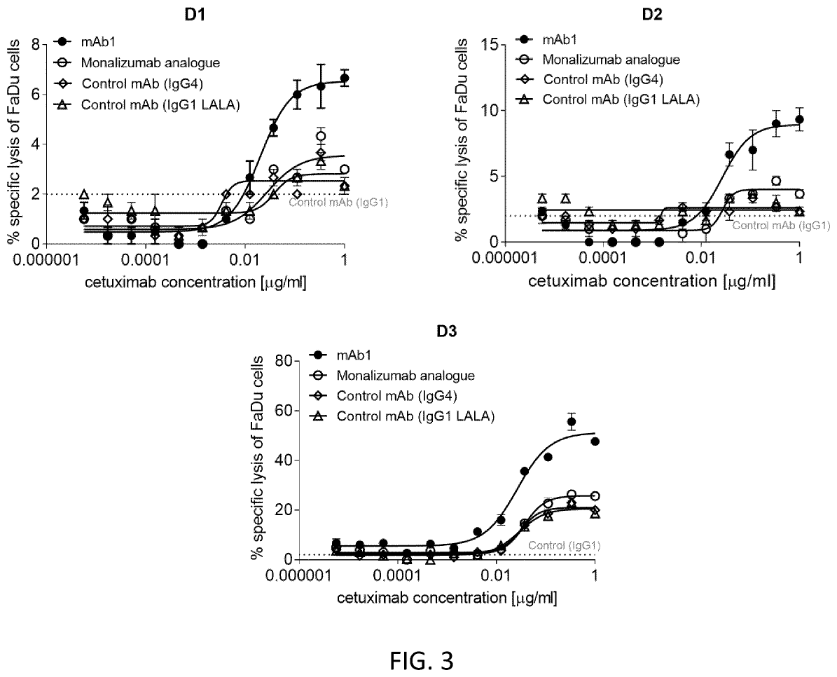

As shown in FIG. 3, mAB1 substantially enhanced cetuximab-induced cytotoxicity. A milder effect was observed when cetuximab was combined with the monalizumab analogue. These data support that mAb1 can potentiate the cytotoxic activity of NK cells mediated by an ADCC-inducing antibody.

### Example 4: MAb1 Potentiates Cetuximab and Futuximab/Modotuximab-Induced ADCC in A431 Cells

This example describes the effect of the combination of mAb1 and ADCC-inducing monoclonal anti-EGFR antibody treatment cetuximab or futuximab/modotuximab on killing of tumor cell lines when co-cultured with primary NK cells.

### Materials and Methods

Human primary NKG2A+NK cells from two healthy human individuals were co-cultured with calcein labeled target cells (A431 HLA-E⁺/EGFR⁺ loaded with HLA-B*0701 peptide) and treated with cetuximab or a combination of futuximab and modotuximab with or without mAb1. Release of calcein was measured after approximately 3 hours and % specific lysis was calculated.

### Results

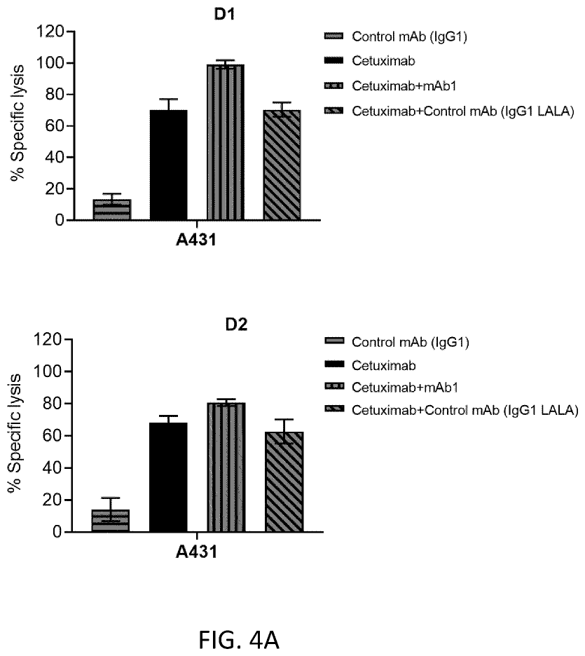

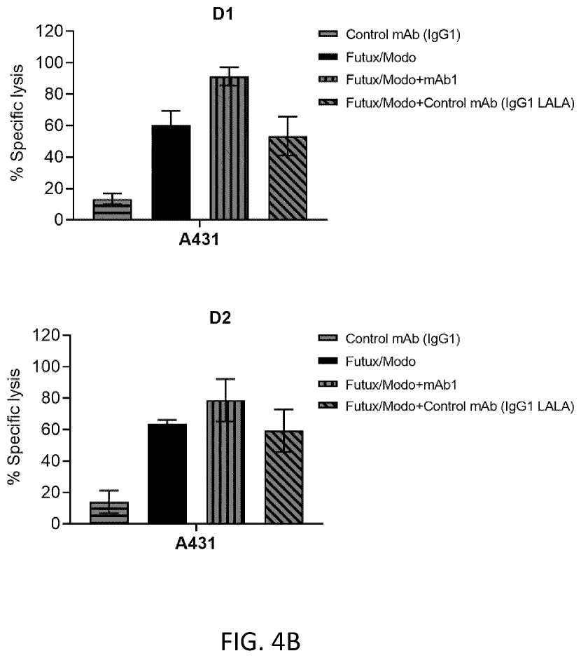

In combination with either cetuximab (FIG. 4A) or a combination of futuximab and modotuximab (FIG. 4B), mAb1 potentiated killing of the tumor cell line A431 over cetuximab or futuximab/modotuximab treatment alone or cetuximab+IgG1 LALA or futuximab/modotuximab+IgG1 LALA.

### Example 5: MAb1 Alone or in Combination with Cetuximab and Futuximab/Modotuximab Induced NK Cell Activation (CD137 Expression)

This example describes the effect of mAb1 tested at one dose, alone or in combination with either cetuximab or futuximab/modotuximab, on inducing activation of primary NK cells in vitro. The expression of CD137 on NK cells as a marker of their activation status was investigated by flow cytometry.

### Materials and Methods

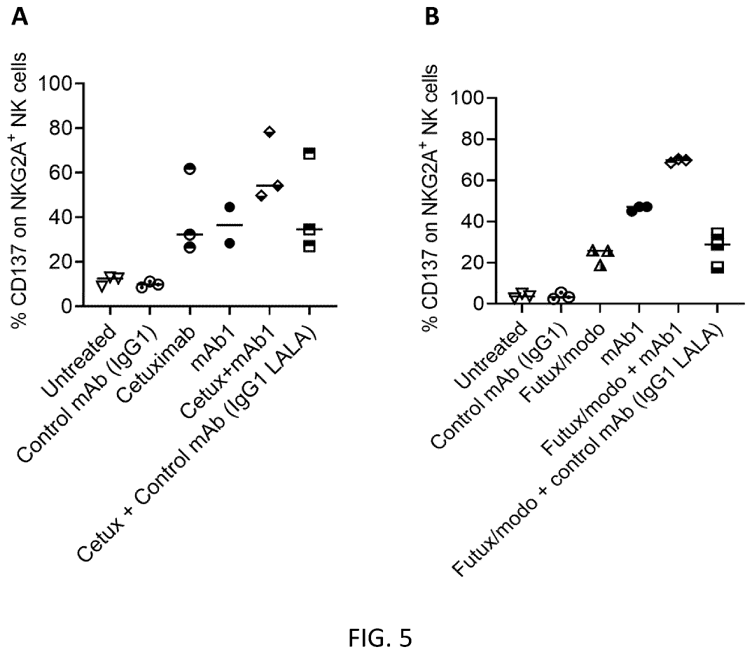

A431 cells (HLA-E+/EGFR+) were HLA-B*0701 peptide-loaded overnight. The next day, NKG2A+NK cells were isolated from fresh PBMCs from three healthy donors and cocultured with the A431 cells at a 10:1 ratio in the presence of 10 ng/mL IL-2 and the antibodies or antibody combinations shown in FIG. 5. After 48 hours of culture, the cells were stained with Zombie Dye Live/Dead stain and anti-FcR antibodies followed by surface staining using anti-CD3 APC-H7 (SK7, BD Biosciences), anti-CD56 BV650 (NCAM-16.2, BD Biosciences), anti-CD16 PE (B73.1, BD Biosciences) and anti-CD137 BV421 (B3, BD Biosciences) antibodies and analyzed by flow cytometry using FACScelesta.

### Results

As shown in FIG. 5, mAb1 as well as cetuximab or futuximab/modotuximab induced NK cell activation alone, but the expression of CD137 was further induced in combinations of mAb1 with cetuximab or futuximab/modotuximab.

### Example 6: MAb1 Alone or in Combination with Futuximab/Modotuximab Induced Secretion of IFNγ by NK Cells

This example describes the effect of mAb1 tested at one dose, alone or in combination with futuximab/modotuximab, on inducing secretion of IFNγ by primary NK cells in vitro. Supernatants from treated cocultures were harvested and analyzed by ELISA for the secretion of IFNγ.

### Materials and Methods

A431 cells (HLA-E+/EGFR+) were pulsed with HLA-B*0701 peptide overnight. The next day, NKG2A+NK cells were isolated from fresh PBMCs from three healthy donors and cocultured with the A431 cells at a 10:1 ratio in the presence of 10 ng/mL IL-2 and anti-NKG2A antibodies. After 48 hours of culture, the cell supernatants were harvested and the concentration of IFNγ was quantified by ELISA (Invitrogen, 88-7316-88).

### Results

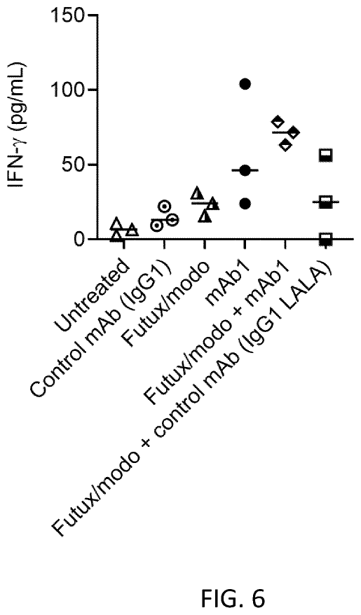

mAb1 and futuximab/modotuximab treatment each stimulated the secretion of IFNγ alone, but IFNγ secretion was further induced when the two were combined (FIG. 6).

### Example 7: MAb1 Potentiates Avelumab-Induced ADCC in A431 and MDA-MB-231 Tumor Cell Lines

This example describes the effect of the combination of mAb1 and the ADCC-inducing monoclonal anti-PD-L1 antibody avelumab on killing of tumor cell lines when co-cultured with primary NK cells.

### Materials and Methods

Human primary NKG2A+NK cells from two healthy human individuals were co-cultured with calcein labeled A431 and MDA-MB-231 tumor cells (both HLA-E+/PD-L1+ loaded with HLA-B*0701 peptide). Release of calcein was measured after approximately 3 hours and % specific lysis was calculated.

### Results

In combination with avelumab, mAb1 potentiated killing of the two tumor cell lines A431 and MDA-MB-231 in both donors compared to avelumab treatment alone or to avelumab+IgG1 LALA.

### Example 8: Mab1 in Combination with Avelumab Induced NK Cell Activation (CD137) and IFNγ Secretion

This example describes the effect of a single dose of mAb1 alone or in combination with avelumab on inducing CD137 expression as well as secretion of IFNγ by primary NK cells in vitro.

### Materials and Methods

A431 cells (HLA-E+/EGFR+) were pulsed with HLA-B*0701 peptide overnight. The next day, NKG2A+NK cells were isolated from fresh PBMCs from healthy donors and cocultured with the A431 cells at a 10:1 ratio in the presence of 10 ng/mL IL-2 and mAb1, avelumab, control antibody IgG1, a combination of mAb1 and avelumab, or a combination of avelumab and control IgG1 LALA. After 48 hours of culture, the cells were stained with Zombie Dye Live/Dead stain and anti-FcR antibodies followed by surface staining using anti-CD3 APC-H7 (SK7, BD Biosciences), anti-CD56 BV650 (NCAM-16.2, BD Biosciences), anti-CD16 PE (B73.1, BD Biosciences) and anti-CD137 BV421 (B3, BD Biosciences) antibodies and analyzed by flow cytometry using FACScelesta. In addition, supernatants from cell cultures were harvested and the concentration of IFNγ was quantified by ELISA (Invitrogen, 88-7316-88).

### Results

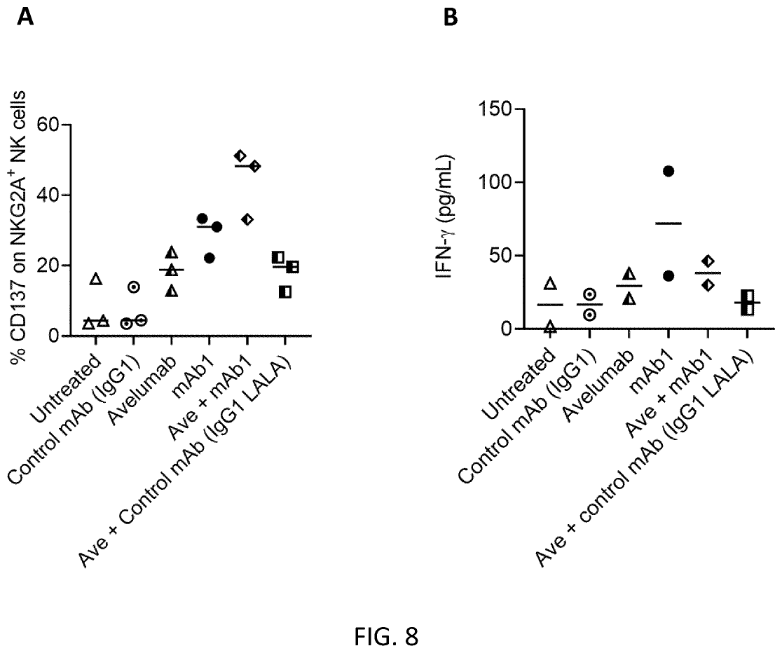

NK-cell activation (as assessed by CD137 expression) was induced by mAb1 alone, but was further induced by the combination of mAb1 and avelumab (FIG. 8A). In addition, IFNγ secretion was induced by mAb1 alone, but was further induced by the combination of mAb1 and avelumab as compared to avelumab treatment alone (FIG. 8B).

### Example 9: In Vivo Tumor Growth Inhibition by mAb1 in Combination with Avelumab

This example demonstrates in vivo efficacy of mAb1 in combination with avelumab on MDA-MB-231 tumor growth in CD34 humanized NOG mice.

### Materials and Methods

Human breast adenocarcinoma cell line MDA-MB-231 was subcutaneously engrafted onto human IL15 boosted NOG mice humanized with human CD34⁺ cord blood stem cells. Tumors were measured three times weekly by caliper in two dimensions and tumor volume in mm³ was calculated per the formula: (width)²×length×0.5. Treatment was initiated at a tumor volume average of 65 mm³. Mice were treated three times weekly for a total of nine treatments by intraperitoneal injection of vehicle, mAb1 (10 mg/kg), avelumab (10 mg/kg) or a combination of mAb1 and avelumab (10 mg/kg per antibody). Two-way ANOVA with Bonferroni's multiple comparisons test was applied to compare tumor volumes at each time-point between treatment groups. Statistical analyses were performed using GraphPad Prism version 9.1.0 (GraphPad Software, Inc.). At study termination, tumors were harvested and analyzed by flow cytometry. Cells were stained with anti-CD45-PE-Cy7, anti-CD3-FITC, anti-CD4-PE, and anti-CD8-APC-Cy7 antibodies, and Zombie Aqua was used for live/dead cell discrimination. Cells were analyzed using a FACSVerse flow cytometer and the FacsDiva Software. Data analyses were performed using GraphPad Prism version 9.1.0 (GraphPad Software, Inc.).

### Results

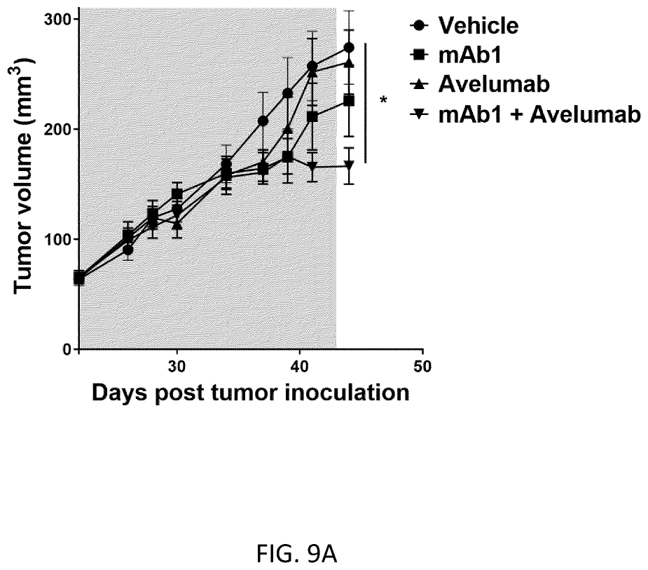

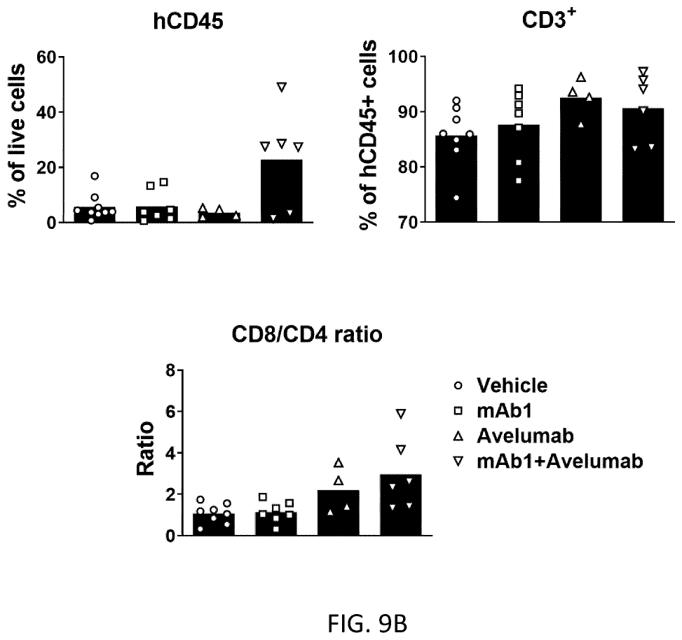

As shown in FIG. 9A, mAb1 combined with avelumab demonstrated an anti-tumor effect on MDA-MB-231 xenograft tumors engrafted in CD34 humanized mice. Treatment induced a significant reduction of tumor growth (P<0.05 vs. vehicle control). Flow cytometer analysis of tumors revealed an increase in infiltration of CD3+ cells with a higher proportion of CD8+ cells compared to CD4+ cells in mice treated with avelumab and the combination of mAb1 and avelumab (FIG. 9B).

### Example 10: Phase 1a/1b Clinical Protocol for mAb1 Monotherapy and Combination Therapy

This example describes a clinical trial protocol for a Phase 1a/1b, open-label, multicenter study investigating the safety, tolerability, and preliminary anti-neoplastic activity of mAb1 (anti-NKG2A) as monotherapy and in combination with mAb3 (anti-PD-1) in patients with advanced solid tumor malignancies. This study also includes an expansion part with triplet combinations of mAb1 and mAb3 and an anti-HER2 mAb or anti-EGFR mAbs (e.g., futuximab/modotuximab) in patients with metastatic gastric or colorectal cancers.

Maximum study duration for each participating patient: each patient will participate in the study until confirmed disease progression, loss to follow-up, an adverse event leading to withdrawal, significant noncompliance with the study protocol, withdrawal of consent, end of study, or death from any cause. For all patients, the maximum duration of the treatment period will not exceed 1 year for patients with confirmed CR and 2 years for patients with confirmed PR. Longer treatment duration might be permitted if patient benefits outweigh the risks according to the investigator's judgment and after consultation with the sponsor.

### Study Design

Each of the four arms of the clinical study is described below.

A. Part 1a—mAb1 Monotherapy

A first primary objective is to assess safety and tolerability of mAb1 as single agent. The corresponding primary endpoints are:
incidence, severity, and relationship of AEs;
AEs leading to dose interruption, modification delays and permanent treatment discontinuation;
changes from baseline to the end of the study in safety laboratory values, vital signs and ECG measurements.

A second primary objective is to determine the maximum tolerated dose (MTD) (or the maximum administered dose [MAD]) of mAb1 as single agent. The corresponding primary endpoints are:
incidence of dose limiting toxicities (DLTs) during Cycle 1, and
incidence and severity of adverse events (AEs).

A first secondary objective is to evaluate the preliminary antitumor activity of mAb1 administered in Cycle 1 followed by mAb3 afterwards per investigator assessment using Response Evaluation Criteria in Solid Tumors (RECIST) v1.1. The corresponding secondary endpoints are:
ORR per investigator assessment of antitumor activity (using RECIST v1.1);
clinical Benefit Rate (CBR) (CR+PR+stable disease (SD) 6 months);
duration of response (DOR);
progression free survival (PFS); and
overall survival (OS).

A second secondary objective is to evaluate the immunogenicity of mAb1. The corresponding secondary endpoint is formation of anti-mAb1 antibodies.

A third secondary objective is to characterize the pharmacokinetic (PK) profile of mAb1. The corresponding secondary endpoint is mAb1 PK parameters including but not limited to area under the plasma concentration-time curve (AUC), Tₘₐₓ, maximum plasma concentration (Cmax) and C_{trough}.

A first exploratory objective is to explore potential pharmacodynamic (PD) biomarkers of activity in tumor biopsies (pre- and post-treatment) and/or peripheral blood. The corresponding exploratory endpoints are:
NKG2A receptor occupancy by dose level in tumor biopsies and/or peripheral blood;
changes in gene expression profiles, immune cell subsets and activation status in tumor biopsies and/or peripheral blood;
changes in HLA-E and NKG2A expression in tumor biopsies and/or peripheral blood; and
changes in plasma concentration of soluble HLA-E and potential other soluble factors.

A second exploratory objective is to explore any potential PK/PD relationships via population modelling. The corresponding exploratory endpoint is the relationship between mAb1 PK and PD parameters in PK/PD models and simulation outcomes.

A third exploratory objective is to assess potential predictive biomarkers of response to mAb1 from baseline tumor and/or peripheral blood samples. The corresponding exploratory endpoints are:
HLA-E expression and NKG2A-positive immune infiltrate in tumor biopsies;
plasma concentration of soluble HLA-E or potential other soluble factors; and
other potential predictive biomarkers in tumor biopsies associated with immunomodulating agents or specific to the anti-NKG2A mechanism of action.
   B. Part 1 b—mAb1+mAb3 Combination Therapy

A first primary objective is to assess safety and tolerability of mAb1 when administered in combination with mAb3. The corresponding primary endpoints are:
incidence, severity, and relationship of AEs;
AEs leading to dose interruption, modification, delays and permanent treatment discontinuations; and
changes from baseline to the end of the study in safety laboratory values, vital signs and ECG measurements.

A second primary objective is to determine the MTD or the MAD and/or the RP2D of mAb1 when administered in combination with mAb3. The corresponding primary endpoints are:
incidence of DLTs during Cycle 1; and
incidence and severity of AEs.

A first secondary objective is to evaluate the preliminary antitumor activity of mAb1 in combination with mAb3 per investigator assessment using RECIST v1.1. The corresponding secondary endpoints are:
ORR per investigator assessment of antitumor activity (using RECIST v1.1);
clinical benefit rate (CBR) (CR+PR+SD 6 months);
DOR;
PFS; and
OS

A second secondary objective is to evaluate the immunogenicity of mAb1 alone or in combination with mAb3. The corresponding secondary objectives are:
formation of anti-mAb1 antibodies; and
formation of anti-mAb3 antibodies.

A third secondary objective is to characterize the PK profile of mAb1 in combination with mAb3 and to investigate a potential PK interaction between mAb1 and mAb3. The corresponding secondary endpoints are:
mAb1 PK parameters including but not limited to AUC, Tₘₐₓ, Cₘₐₓ and C_{trough}; and
external visual predictive check (VPC) performed on the individual concentrations of mAb3 using an internal population PK model.

A first exploratory objective is to explore potential PD biomarkers of activity in combination with mAb3 in tumor biopsies (pre- and post-treatment) and peripheral blood. The corresponding exploratory endpoints are:
NKG2A receptor occupancy by dose level in tumor biopsies and/or peripheral blood;
changes in gene expression profiles, immune cell subsets and activation status in tumor biopsies and/or peripheral blood;
changes in HLA-E and NKG2A expression in tumor biopsies and/or peripheral blood; and
changes in plasma concentration of soluble HLA-E and potential other soluble factors.

A second exploratory objective is to explore the relationship between PD-L1 tumor status (CPS) and response. The corresponding exploratory endpoints are:
PD-L1 expression in tumor biopsies; and
relationship between PD-L1 expression in tumor biopsies and patient response.

A third exploratory objective is to explore any potential PK/PD relationships via population modelling that may support selection of the RP2D. The corresponding exploratory endpoint is the relationship between mAb1 PK and PD parameters in PK/PD models and simulation outcomes.

A four exploratory objective is to assess potential predictive biomarkers of response to mAb1 in combination with mAb3 from baseline tumor and peripheral blood samples. The corresponding exploratory endpoints are:
HLA-E expression and NKG2A positive immune infiltrate in tumor biopsies;
plasma concentration of soluble HLA-E or other potential soluble factors;
and
other potential predictive biomarkers associated to immunomodulating agents or specific to the anti-NKG2A mechanism of action in tumor biopsies.
   C. Part 2a—mAb1+mAb3+Margetuximab Triplet Combinations

In this arm, margetuximab, an anti-HER2 monoclonal antibody, is added to the combination therapy. A primary objective is to evaluate the antitumor activity and efficacy of the triplet combination (mAb1+mAb3+margetuximab) in HER2-positive patients with locally advanced unresectable or metastatic gastric cancer. The corresponding primary endpoint is ORR per investigator assessment of antitumor activity using RECIST v1.1.

A first secondary objective is to assess the safety and tolerability profile of mAb1 in combination with mAb3 and margetuximab. The corresponding secondary endpoints are:
incidence and severity of AEs;
AEs leading to dose interruption, modification, delays, and permanent treatment discontinuation; and
changes from baseline to the end of the study in safety laboratory values, vital signs, and ECG measurements.

A second secondary objective is to confirm the RP2D of mAb1 in combination with mAb3 and margetuximab. The corresponding secondary endpoint is overall safety profile, PK profile and relationship between exposure and PD (i.e., safety, efficacy, and biomarkers).

A third secondary objective is to evaluate additional efficacy parameters to assess antitumor activity of mAb1 in combination with mAb3 and margetuximab. The corresponding secondary endpoints are CBR, DOR, PFS, and OS.

A fourth secondary objective is to characterize the PK profile of mAb1 in combination with mAb3 and margetuximab and to investigate a potential PK interaction between mAb1, mAb3 and margetuximab. The corresponding secondary endpoints are:
mAb1 PK parameters including but not limited to AUC, Tₘₐₓ, Cₘₐₓ and C_{trough},
external VPC performed on the individual concentrations of mAb3 using an internal population PK model; and
external VPC performed on the individual concentrations of margetuximab using a literature population PK model.

A fifth secondary objective is to evaluate the immunogenicity of mAb1 in combination with mAb3 and margetuximab. The corresponding secondary endpoints are:
formation of anti-mAb1 antibodies;
formation of anti-mAb3 antibodies; and
formation of anti-margetuximab antibodies.

A sixth secondary objective is to explore the relationship between PD-L1 (CPS) or HER2 tumor status and response. The corresponding secondary endpoints are:
PD-L1 and HER2 status in tumor biopsies;
relationship between PD-L1 expression in tumor biopsies and patient response; and
correlation between HER2 expression/amplification status in tumor biopsies and patient response.

A first exploratory objective is to further explore potential PD biomarkers of activity in combination with mAb3 and margetuximab and their relationship with PK and/or anti-tumor activity. The corresponding exploratory endpoints are:
NKG2A receptor occupancy in tumor biopsies and/or peripheral blood;
changes in gene expression profiles, immune cell subsets and activation status in tumor biopsies and/or peripheral blood;
changes in HLA-E and NKG2A expression in tumor biopsies and/or peripheral blood;
plasma concentration of soluble HLA-E and potential other soluble factors; and
correlation between PD biomarkers and PK and/or patient response.

A second exploratory objective is to assess predictive biomarkers of response to mAb1 in combination with mAb3 and margetuximab from baseline tumor and peripheral blood samples and their relationship with antitumor activity. The corresponding exploratory endpoints are:
HLA-E expression and NKG2A positive immune infiltrate in tumor biopsies;
plasma concentration of soluble HLA-E or potential other soluble factors;
other potential predictive biomarkers associated to immunomodulating agents or specific to the anti-NKG2A mechanism of action in tumor biopsies; and
correlation between potential predictive biomarkers and patient response.
   D. Part 2b—Mab1+Mab3+Futuximab/Modotuximab Triplet Combination

A primary objective is to evaluate the antitumor activity and efficacy of the triplet combination (mAb1+mAb3+futuximab/modotuximab) in patients with metastatic colorectal cancer. The corresponding primary endpoint is ORR per investigator assessment of antitumor activity using RECIST v1.1.

A first secondary objective is to assess the safety and tolerability profile of mAb1 in combination with mAb3 and futuximab/modotuximab. The corresponding secondary endpoints are:
incidence and severity of AEs;
AEs leading to dose interruption, modification, delays, and permanent treatment discontinuation; and
changes from baseline to the end of the study in safety laboratory values, vital signs, and ECG measurements.

A second secondary objective is to confirm the RP2D of mAb1 in combination with mAb3 and futuximab/modotuximab. The corresponding secondary endpoint is overall safety profile, PK profile and relationship between exposure and PD (i.e. safety, efficacy, and biomarkers).

A third secondary objective is to evaluate additional efficacy parameters to assess antitumor activity of mAb1 in combination with mAb3 and futuximab/modotuximab. The corresponding secondary endpoints are CBR, DOR, PFS, and OS.

A fourth secondary objective is to characterize the PK profile of mAb1 in combination with mAb3 and futuximab/modotuximab and to investigate a potential PK interaction between mAb1, mAb3 and futuximab/modotuximab. The corresponding secondary endpoints are:
mAb1 PK parameters including but not limited to AUC, Tₘₐₓ, Cₘₐₓ, and C_{trough},
external VPC performed on the individual concentrations of mAb3 using an internal population PK model; and
external VPC performed on the individual concentrations of futuximab/modotuximab PK using an internal population PK model.

A fifth secondary objective is to To evaluate the immunogenicity of mAb1 in combination with mAb3 and futuximab/modotuximab. The corresponding secondary endpoints are:
formation of anti-mAb1 antibodies;
formation of anti-mAb3 antibodies; and
formation of anti-futuximab/modotuximab antibodies

A sixth secondary objective is to explore the relationship between PD-L1 tumor status (CPS) and response. The corresponding secondary endpoints are:
PD-L1 status in tumor biopsies; and
correlation between PD-L1 expression in tumor biopsies and patient response.

A first exploratory objective is to further explore potential PD biomarkers of activity in combination with mAb3 and futuximab/modotuximab and their relationship with PK and/or anti-tumor activity. The corresponding exploratory endpoints are:
NKG2A receptor occupancy in tumor biopsies and/or peripheral blood;
changes in gene expression profiles in tumor biopsies and/or peripheral blood;
changes in immune cell subsets and activation status in tumor biopsies and peripheral blood;
changes in HLA-E and NKG2A expression in tumor biopsies and/or peripheral blood;
plasma concentrations of soluble HLA-E and potential other soluble factors; and
correlation between PD biomarkers and PK and/or patient response.

A second exploratory objective is to assess predictive biomarkers of response to mAb1 in combination with mAb3 and futuximab/modotuximab from baseline tumor and peripheral blood samples and their relationship with anti-tumor activity. The corresponding exploratory endpoints are:
HLA-E expression and NKG2A positive immune infiltrate in tumor biopsies;
plasma concentration of soluble HLA-E or potential other soluble factors;
other potential predictive biomarkers associated to immunomodulating agents or specific to the anti-NKG2A mechanism of action in tumor biopsies; and
correlation between potential predictive biomarkers and patient response.

### Methodology

This study determines the safety and antitumor activity of mAb1 (anti-NKG2A) in combination with mAb3 (anti-PD1) in patients with advanced solid tumour malignancies (in Part 1) and in triplet combinations with margetuximab or futuximab/modotuximab (in Part 2) in two disease specific cohorts.

To establish the MTD or MAD of mAb1 alone and in combination with mAb3, the safety of mAb1 monotherapy will first be evaluated in Part 1a and then in combination with mAb3 in Part 1b. The RP2D defined in Part 1b will be used in combination with margetuximab orfutuximab/modotuximab in the dose expansion part (Part 2). The RP2D may be chosen at any time during Part 1b based on the overall safety profile including PK/PD whether or not the MTD is characterized. The RP2D will not exceed the MTD (if characterized).

The trial will begin with an mAb1 monotherapy dose escalation starting at the 20 mg dose (Part 1a). The DLT observation period will be 28 days (Cycle 1). Upon completion of DLT evaluations period at the second dose level (100 mg) and deemed safe by the Safety Review Committee (SRC) upon evaluation of safety, the Part 1b combination mAb1 (starting at 100 mg)+mAb3 (200 mg) dose escalation will be initiated. After this, escalation to the next mAb1 monotherapy dose level (300 mg) and beyond in Part 1a will run concurrently with combination dose escalation in Part 1b. There are 5 planned dose levels for the monotherapy dose escalation (20, 100, 300, 750, 1500 mg) plus a dose de-escalation level of 8 mg, if needed. There are 4 planned dose levels for the combination dose escalation (100, 300, 750, 1500 mg) plus a dose de-escalation level of 20 mg, if needed. mAb3 will be used as a fixed dose of 200 mg in the Part 1b combination dose escalation (see Table 2).

TABLE 2
Summary of Dosing in Part I

| Part 1a (mAb1 Monotherapy) | | Part 1b (mAb1 + mAb3 Combination Therapy) | | |
|---|---|---|---|---|
| Dose | mAb1 Dose | Dose | mAb1 Dose | mAb3 Dose |
| Level | (mg) | Level | (mg) | (mg) |
| DL5 | 1500 | DL4 | 1500 | 200 |
| DL4 | 750 | DL3 | 750 | |
| DL3 | 300 | DL2 | 300 | |
| DL2 | 100 | DL1 | 100 | |
| DL1 | 20 | DL-1 | 20 | |
| DL-1 | 8 | | | |

All patients in the Part 1a mAb1 monotherapy dose escalation cohorts will receive mAb3 (200 mg) monotherapy after completing one cycle of mAb1 and finishing the DLT evaluation period. These patients will continue to receive mAb3 monotherapy until confirmed disease progression, unacceptable toxicity or until 12 months following a confirmed CR or 24 months following a confirmed PR.

To optimize signal of activity, triplet combinations will be initiated as expansion cohorts (Parts 2a and 2b) using the RP2D of doublet (mAb1+mAb3) in indications with unmet medical need (i.e., metastatic colorectal cancer and locally advanced unresectable or metastatic HER2+ gastric cancer) and with compounds potentially enhanced by mAb1 such as monoclonal antibodies that induce ADCC (e.g., anti-HER2 margetuximab or other anti-HER2 mAbs and anti-EGFR mAbs futuximab/modotuximab). The RP2D of the mAb1+mAb3 combination will be combined with margetuximab (Part 2a) or anti-EGFR (futuximab/modotuximab) (Part 2b).

### Part 1 (Dose Escalation)

Part 1a will initiate with a single-patient dose escalation (accelerated titration). The accelerated titration design will be switched to a Bayesian optimal interval (BOIN) design if the patient experiences an mAb1-related Grade 2 AE or a DLT. A maximum of 3 patients could be recruited as back-filled patients to generate additional data to further define/characterize the RP2D and PK/PD relationship. Doses of mAb1 that will be administered to backfilled patients will be determined by the sponsor in agreement with SRC and will not be higher than the MTD; these cohorts will follow the Part 1a treatment schedule and will not be subject to a DLT window. Data from back-fill patients will not serve DLT analysis but will be accounted for in the overall safety analysis.

In Part 1 b, de-escalation to 20 mg mAb1 or intermediate dose levels as well as higher dose levels may be explored. Patients at each mAb1 dose level will receive their mAb1 dose in combination with mAb3 (200 mg).

During Part 1, dosing will be staggered by at least 24 hours between all patients in each dose level. The first patient dosed in each dose level will be observed for safety for 24 hours period, i.e., If no safety issues are noted, then the 2nd patient can be enrolled in the same dose level and the 3rd after another 24 hours of safety observation. All patients enrolled in Parts 1a and 1b must have advanced or metastatic disease.

Other objectives of the study include preliminary assessment of the antitumor activity of mAb1 in combination with mAb3, evaluation of immunogenicity, characterization of the PK profile of mAb1 alone and in combination with mAb3, and evaluation of potential pharmacodynamic markers supporting proof of mechanism and other predictive biomarkers in both blood and tumor tissue (tumor biopsies are optional in Part 1a, except for back-filled patients, and are mandatory in Part 1b.

### Part 2 (Dose Expansion)

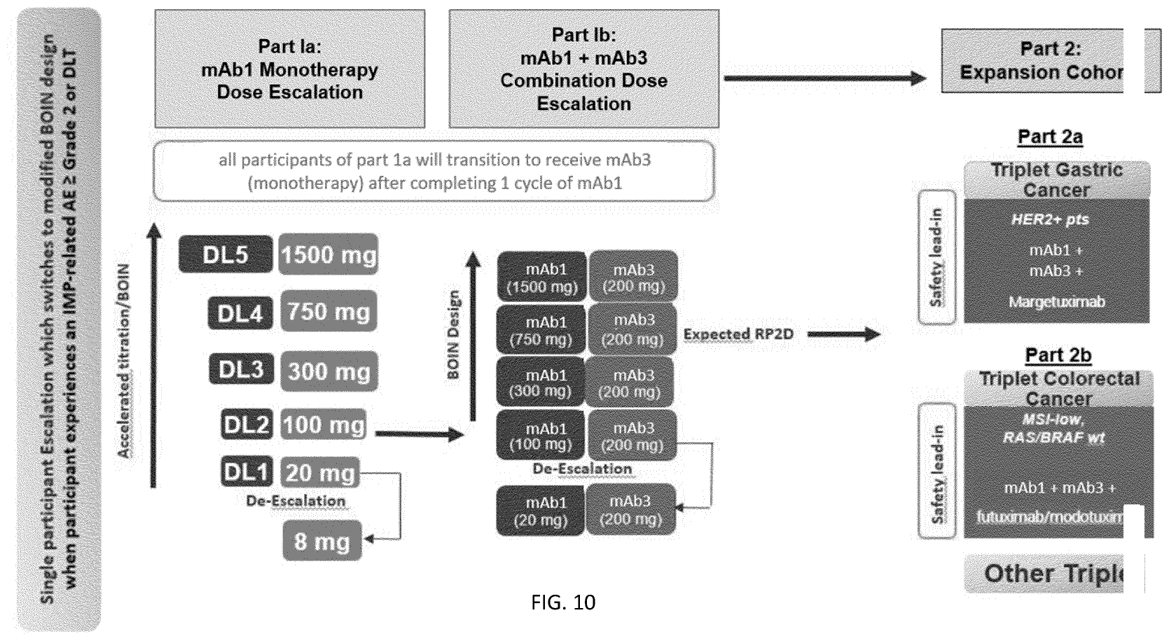

Part 2 will be conducted in 2 expansion cohorts (FIG. 10): (i) Part 2a in patients with HER2-positive metastatic gastric cancer with the addition of a third component, an anti-HER2 therapy (margetuximab); and (ii) Part 2b in patients with metastatic colorectal cancer with the addition of a third component, an anti-EGFR therapy (futuximab/modotuximab).

Part 2a will be conducted in HER-2 positive gastric cancer patients who have failed first-line standard of care therapy including cytotoxic chemotherapy and trastuzumab and pembrolizumab and are fit to initiate a second line. In patients with colorectal cancer (Part 2b), third and fourth lines are permitted if the patients received standard of care therapies previously.

### Part I Screening Criteria

The patients are male or female patient aged 18 years of age. The medical and therapeutic criteria include:
patients with histologically or cytologically confirmed unresectable, locally advanced or metastatic solid tumor malignancies;
patients with a malignancy that is not currently amenable to surgical intervention due to either medical contraindications or non-resectability of the tumor;
patients with measurable disease according to RECIST v1.1 and must have had radiologic assessment of progression on the prior therapy before study entry;
patients refractory to or intolerant of existing therapy(ies) known to provide clinical benefit or considered standard of care;
estimated life expectancy 12 weeks;
ECOG (Eastern Cooperative Oncology Group) performance status 0 or 1; and
documented radiographic progression on prior line of therapy.

The inclusion criteria include:
adequate hematological function based on the last assessment performed within 7 days prior to the first Investigational Medicinal Products (IMP) administration, defined as: absolute neutrophil count (ANC) 1.5×10⁹/L; hemoglobin 8 g/dL (in case of blood transfusion, the hemoglobin assessment must be performed 2 weeks or more after the transfusion); platelet count 75×10⁹/L; and adequate coagulation function for all patients (for patients receiving anti-coagulant therapy (except platelet anti-aggregates) the adequate therapeutic levels of INR should be confirmed);
adequate renal function based on the last assessment performed within 7 days prior to the first IMP administration defined as: creatinine clearance 40 mL/min., assessed using the Cockcroft & Gault formula; and
adequate hepatic function based on the last assessment performed within 7 days prior to the first IMP administration, defined as: total serum bilirubin ≤1.5×upper limit of normal (ULN) (unless Gilbert disease confirmed), and aspartate aminotransferase (AST) and alanine aminotransferase (ALT)≤3×ULN (unless if liver function abnormalities are due to underlying liver metastasis, AST and ALT s 5×ULN).

Exclusion criteria include:
Patients with any other unresolved Grade >1 toxicity associated with prior anti-neoplastic therapy with the exception of persistent Grade 2 alopecia orvitiligo, peripheral neuropathy and/or endocrine end-organ failure being adequately managed by hormone replacement therapy;
major surgery within 4 weeks prior to the first IMP administration or patients who have not recovered from side effects of the surgery;
patients with any other serious/active/uncontrolled infection, any infection requiring parenteral antibiotics, within 2 weeks prior to first IMP administration;
active Hepatitis B Virus infection determined as HBsAg positive or active Hepatitis C Virus infection determined as detection of HCV RNA in serum or plasma by a sensitive quantitative molecular method;
carriers of HIV antibodies (patients with controlled HIV RNA are allowed if stable on medication and undetectable per standard management);
patients with a history of organ transplantation (e.g., stem cell or solid organ transplant);
patients with active thrombosis, or a history of deep vein thrombosis or pulmonary embolism, within 4 weeks prior to first IMP administration, unless adequately treated and considered by the investigator to be
stable;
patients with active uncontrolled bleeding or a known bleeding diathesis;
patients with a known clinically significant cardiovascular disease or condition, including: need for anti-arrhythmic medical therapy for a ventricular arrhythmia or other uncontrolled arrhythmia (patients with controlled atrial fibrillation (heart rate <90) for >30 days prior to inclusion are eligible); severe conduction disturbance (e.g. 3rd degree heart block); uncontrolled hypertension; congestive heart failure currently requiring therapy; Class Ill or intravenous (IV) cardiovascular disease according to the New York Heart Association
(NYHA) Functional Classification; history of acute coronary syndromes (including myocardial infarction and unstable angina), coronary
angioplasty, stenting, or bypass grafting within 6 months;
history of gastrointestinal perforation, or intra-abdominal abscess within 28 days of inclusion; history of cirrhosis (defined as a known diagnosis of Child Pugh B or C) or chronic liver condition; history of pulmonary fibrosis or relevant uncontrolled chronic pulmonary condition; patients with non-healing wounds on any part of the body;
patients who have received prior: small molecule inhibitors, and/or other similar investigational agent: ≤2 weeks or 5 half-lives, whichever is shorter, chemotherapy, other monoclonal antibodies, antibody-drug conjugates, or other similar experimental therapies ≤3 weeks or 5 half-lives, whichever is shorter, or radioimmunoconjugates or other similar experimental therapies ≤6 weeks or 5 half-lives, whichever is shorter;
patients must have recovered from any AE (from previous anti-cancer therapy) to Common Terminology Criteria for Adverse Events (CTCAE) V5.0 Grade 1 or lower, Grade 2 neuropathy is acceptable (patients
receiving replacement hormone therapy due to previous AEs will not be excluded from participation in this study if the associated AE has recovered to Grade 1 with replacement therapy prior to first administration on study;
patients who have received anti-NKG2A mAb in the past;
patients with known, untreated central nervous system (CNS) or leptomeningeal metastases, or spinal cord compression; patients with any of these not controlled by prior surgery or radiotherapy, or patients with symptoms suggesting CNS involvement for which treatment is required (only patients with CNS metastases clinically and radiographically stable for 4 weeks and with low corticosteroids treatment are allowed to participate (≤10 mg/day prednisone or equivalent is permitted if being administered for 4 weeks);
other malignancies including those which were radically treated and for which the remission period at the time of screening is less than five years. Exemptions for this minimally required duration of remission period may be applied for carcinoma in situ of the cervix and basal cell skin cancer that are deemed to be cured by adequate treatment;
treatment with systemic immunosuppressive therapy (except steroids given in prophylactic setting or at a chronic low dose [≤10 mg/day prednisone equivalent]), inhaled, intranasal, intraocular, topical &
intraarticular joint injections are allowed;
prior radiotherapy if completed less than 4 weeks before first IMP administration, except if provided as a short course for symptoms palliation only;
active autoimmune disease that might deteriorate when receiving an immunostimulatory agents (patients with type I diabetes mellitus, vitiligo, psoriasis, hypo- or hyperthyroid disease not requiring
immunosuppressive treatment are eligible);
administration of a live vaccine within 28 days prior to inclusion; and
known prior severe hypersensitivity to investigational products or any component in their formulations, including known severe hypersensitivity reactions to monoclonal antibodies (NCI CTCAE v. 5.0 Grade 3).

### Part 2a Screening Criteria

The medical and therapeutic criteria include:
patients having histologically proven unresectable locally advanced or metastatic HER2⁺ cancer determined as 3+ by IHC or 2+ by IHC and in situ hybridization- (ISH-)amplified (2.0) in tumor biopsy collected at
time of screening;
patient is eligible for second line and must have received treatment with first line of standard therapy including cytotoxic chemotherapy and trastuzumab and pembrolizumab;
patients who have had prior checkpoint inhibitor (CPI) treatment in the past 6 months must have documented confirmed radiographic progression as per iRECIST from it prior to study entry;
patients with measurable disease according to RECIST v1.1 and must have had radiologic assessment of progression on the prior therapy before study entry;
estimated life expectancy 12 weeks.
ECOG performance status 0 or 1.

The inclusion criteria include:
adequate hematological function based on the last assessment performed within 7 days prior to the first IMP administration, defined as: ANC 1.5×10⁹/L, hemoglobin 8 g/dL, platelet count 75×10⁹/L, adequate coagulation function for all patients (for patients receiving anti-coagulant therapy (except platelet anti-aggregates) that affect INR levels, the adequate therapeutic levels of INR should be confirmed);
adequate renal function based on the last assessment performed within 7 days prior to the first IMP administration defined as creatinine clearance ≥30 mL/min, assessed using the Cockcroft & Gault formula; and
adequate hepatic function based on the last assessment performed within 7 days prior to the first IMP administration, defined as: total serum bilirubin <1.5×ULN (unless Gilbert disease confirmed), AST and alanine aminotransferase (ALT)≤2.5×ULN (unless if liver function abnormalities are due to underlying liver metastasis, AST and ALT ≤5×ULN).

The exclusion criteria include those listed above Part 1, plus:
left ventricle ejection fraction (LVEF)<50% by echocardiogram or multi-gated acquisition scan; and
any contraindication present in the margetuximab prescribing information.

### Part 2b Screening Criteria

The medical and therapeutic criteria include:
patients must have histologically or cytologically confirmed adenocarcinoma of metastatic colorectal cancer (mCRC) (all other histological types are excluded), not amenable to surgical intervention due to either medical contraindications or non-resectability of the tumor and with microsatellite instability status as low per institutional guidelines or guidelines from the College of American Pathologists;
based on ctDNA screening blood test analysis patients should be: (i) without RAS (KRAS and NRAS) mutations in any of the following codons: codons 12 and 13 in exon 2, codons 59 and 61 in exon 3, and codons 117 and 146 in exon 4; and (ii) without BRAF V600E mutation;
patients who have had prior checkpoint inhibitor (CPI) treatment in the past 6 months must have documented confirmed radiographic progression as per iRECIST from it prior to study entry;
patients with measurable disease according to RECIST v1.1 and must have had radiologic assessment of progression on the prior therapy before study entry;
estimated life expectancy 12 weeks; and
ECOG performance status 0 or 1.

The inclusion criteria include:
adequate hematological function based on the last assessment performed within 7 days prior to the first IMP administration, defined as: ANC 1.5×10⁹/L, hemoglobin 8 g/dL (in case of blood transfusion, the hemoglobin assessment must be performed 2 weeks or more after the transfusion), platelet count 75×10⁹/L, and adequate coagulation function for all patients (for patients receiving anti-coagulant therapy (except platelet anti-aggregates) the adequate therapeutic levels of INR should be confirmed);
adequate renal function based on the last assessment performed within 7 days prior to the first IMP administration defined as creatinine clearance 30 mL/min., assessed using the Cockcroft & Gault formula;
adequate hepatic function based on the last assessment performed within 7 days prior to the first IMP administration, defined as: total serum bilirubin <1.5×ULN (unless Gilbert disease confirmed), and AST and alanine aminotransferase (ALT)≤3.0×ULN (unless if liver function abnormalities are due to underlying liver metastasis, AST and ALT ≤5×ULN); and
serum potassium, serum phosphates, serum magnesium within normal limits with or without supplementation based on the last assessment performed within 7 days prior to the first IMP administration.

The exclusion criteria include:
patients with a significant gastrointestinal abnormality, including: diarrhea of Grade >1 at the time of inclusion and requirement for IV alimentation;
patients with skin rash of Grade >1 from prior anti-EGFR at the time of inclusion, or any other skin toxicity precluding participation in the study according to investigator's discretion; and
known or suspected hypersensitivity to any of the excipients of formulated futuximab/modotuximab, cetuximab or panitumumab (Grade 3 or 4 hypersensitivity reactions during prior therapy with either cetuximab or panitumumab).

### Test Drugs

The drugs tested in the present clinical study are shown in Table 3 below:

TABLE 3
Investigational Medicinal Products (IMPs)

| | |
|---|---|
| Study Part | IMPs Administered |

Dose Escalation

| | |
|---|---|
| Part 1a | mAb1 for one cycle, then switch to mAb3 monotherapy |
| Part 1b | mAb1 + mAb3 |

Dose Expansion

| | |
|---|---|
| Part 2a | mAb1 + mAb3 + margetuximab |
| Part 2b | mAb1 + mAb3 + futuximab/modotuximab |

### Part 1 Drugs

mAb1 will be administered at the dosage indicated in Table 2 above, or adjusted as needed. Administration will be via IV infusion every 2 weeks (Q2W) (±1 day) on Day 1 and D15 of each 28-day cycle. This 4-week (28-day) period will constitute one treatment cycle. mAb1 will be infused over approximately 30 minutes. The duration of infusion may be extended by 30 minutes or longer if indicated in the event of an infusion-related reaction (IRR). If a patient experiences an IRR during any cycle, the observation period should be extended to 2 hours during that cycle and all subsequent cycles for that patient.

mAb3 will be administered at the dose of 200 mg as an IV infusion every 2 weeks from C2D1 in Part 1a or from C1 D1 in Part 1 b. When co-administered with mAb1 in Part 1 b, mAb1 will be administered first.

### Part 2 Drugs

The administration of mAb1 and mAb3 is as discussed above for Part 1.

For Part 2a, margetuximab will be administered at 15 mg/kg every 3 weeks as a 120-minute (2-hour) IV infusion for the initial dose, then over a minimum of 30 minutes for all subsequent doses. Margetuximab should not be administered as an IV push or bolus. The administration will be through an IV line containing a sterile, nonpyrogenic, low protein binding polyethersulfone (PES) 0.2 micron in-line or add-on filter. After the first 12 weeks, the investigator may decide to administer margetuximab on a Q4 week schedule.

For Part 2b, futuximab/modotuximab will be administered at a dose of 9 mg/kg on Cycle 1 Day 1 (C1D1) (loading dose) and then at a dose of 6 mg/kg weekly (±2 days) beginning on C1D8 (maintenance doses) for all subsequent administrations, by IV infusion. Dose adjustments should be made in the event of noted weight change (±10%) as measured at the beginning of dosing cycle (CxD1). After the first 12 weeks, the investigator may decide to administer futuximab/modotuximab on a Q2W schedule.

### Criteria for Evaluation

Standard response criteria will be applied for disease assessments and response evaluations (RECIST v1.1 and iRECIST v1). Assessments should be performed at the intervals specified in the investigation schedules and in the event of suspected progressive disease. The same method(s) of disease evaluation and the same technique should be used throughout the study as the baseline. Response will be assessed by the investigator or qualified designee and will be noted at each evaluation point as CR, PR, SD, PD, or not evaluable (NE). Disease assessments will be performed at baseline, at the end of cycle 2 and at the end of even-numbered cycles thereafter (approximately every 8 weeks unless a delay is required due to a delay in dosing) and at the time of disease progression.

Per RECIST v1.1, response should be confirmed by repeat imaging assessment not less than 4 weeks from the date the response was first documented. The scan for confirmation of the response may be performed at the earliest 4 weeks after the first documentation of response or at the next scheduled scan (8-week interval from last scan), whichever is clinically indicated. iRECIST will be used by the investigator to assess tumor response and progression after initial radiographic progression per RECIST v.1; treatment decisions may be made accordingly.

The investigator should consider all lesions (target and non-target) in assessing the tumor burden at repeat imaging prior to decide whether to continue treatment. Patients will continue treatment (unless clinically unstable) until a subsequent (at least 4 weeks after the initial assessment of progression) radiographic confirmation of that progression since immune-related pseudo progression is possible. Patients who are clinically unstable are not required to undergo repeat imaging for confirmation of progressive disease.

AEs will be graded using the NCI CTCAE version 5.0. A DLT is defined as an AE, occurring during the 28-day DLT observation period, assessed as unrelated to disease progression, intercurrent illness, or concomitant medications or other etiology, considered as related to the IMP.

Pharmacokinetic measurements include: individual PK parameters such as AUC or Cmax will be derived using a population PK modelling approach, and exploratory assessment of the relationship between individual PK parameters and PD endpoints for efficacy or safety may be performed.

The pharmacodynamic assessments are: NKG2A receptor occupancy assessment and immunophenotyping in periphery, gene expression signature of target engagement in periphery, soluble HLA-E and potential other soluble factors, and tumor biopsies for pharmacodynamic and predictive biomarker assessments.

### Example 11: In Vivo Efficacy of mAb 1 in Combination with Anti-PD1 in Syngeneic MC38-HLAE Tumor Model, in hNKG2a/hCD94 KI Mice

This example demonstrates in vivo efficacy of mAb 1 in combination with anti-PD1 mAb3 in hNKG2A/hCD94 KI mice (Biocytogen, China) engrafted with murine MC38-HLAE colon cancer cells.

### Materials and Methods

Murine MC38-HLAE colon carcinoma cell line was subcutaneously engrafted onto a human NKG2A/CD94 double knock-in mice model (Biocytogen, China). Treatment was initiated with tumor volume of 60 mm3 and the mice were treated three times weekly with a total of nine treatments by intraperitoneal injection of vehicle buffer, mAb 1, anti-PD1 mAb3 or a combination of mAb 1 and anti-PD1 mAb3 (n=10/group). All mAbs were dosed at 10 mg/kg. Tumors were measured three times weekly by caliper in two dimensions and tumor volume in mm³ was calculated per the formula: (width)²×length×0.5.

Two-way ANOVA with Bonferroni's multiple comparisons test was applied to compare tumor volumes at each time-point between treatment groups.

### Results

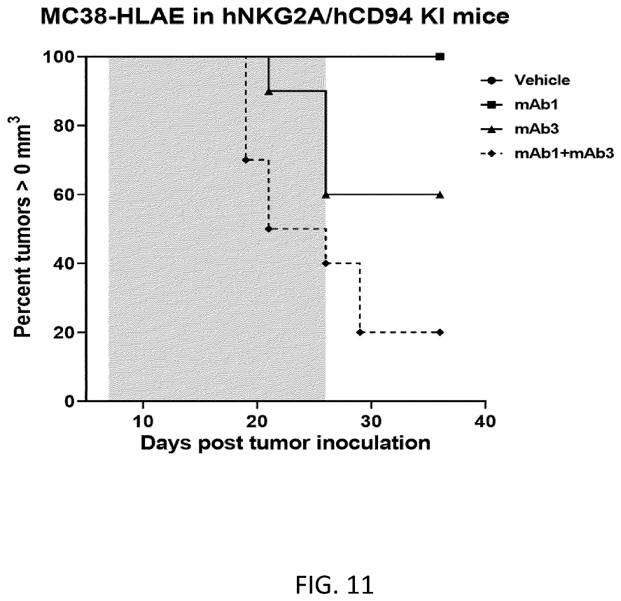

As shown in FIG. 11 combination treatment with mAb1 and anti-PD1 mAb3 induced more, and earlier tumor eradication compared to anti-PD1 mAb3 single treatment.

### Example 12: Titration of Trastuzumab Using MIP-1b

This example describes the effect of addition of mAb1 to titrated trastuzumab on the expression of the pro-inflammatory cytokine MIP-1β of primary NK cells in vitro.

### Materials and Methods

WT SKOV3 cells were pulsed with HLA-B*0701 peptide overnight. The next day, these cells were cocultured with NKG2A+NK cells isolated from fresh PBMCs at a 10:1 ratio in the presence of 10 ng/ml IL-2 and anti-NKG2A antibodies. Data show MIP-1β levels in co-cultures with a single donor. Similarly, N87, BxPC3, SKOV3, A375, A549 and JIMT-1 cells transduced with HLA-E for stable surface expression of HLA-E were cocultured with NKG2A+NK cells isolated from fresh PBMCs at a 10:1 ratio in the presence of 10 ng/ml IL-2 and anti-NKG2A and/or anti-HER2 antibodies or control antibodies. Data show MIP-1β levels in co-cultures with a single donor. After 48 hours of culture, the supernatants were collected and the concentration of MIP-13 was quantified by ELISA (Invitrogen, 88-7034-88).

### Results

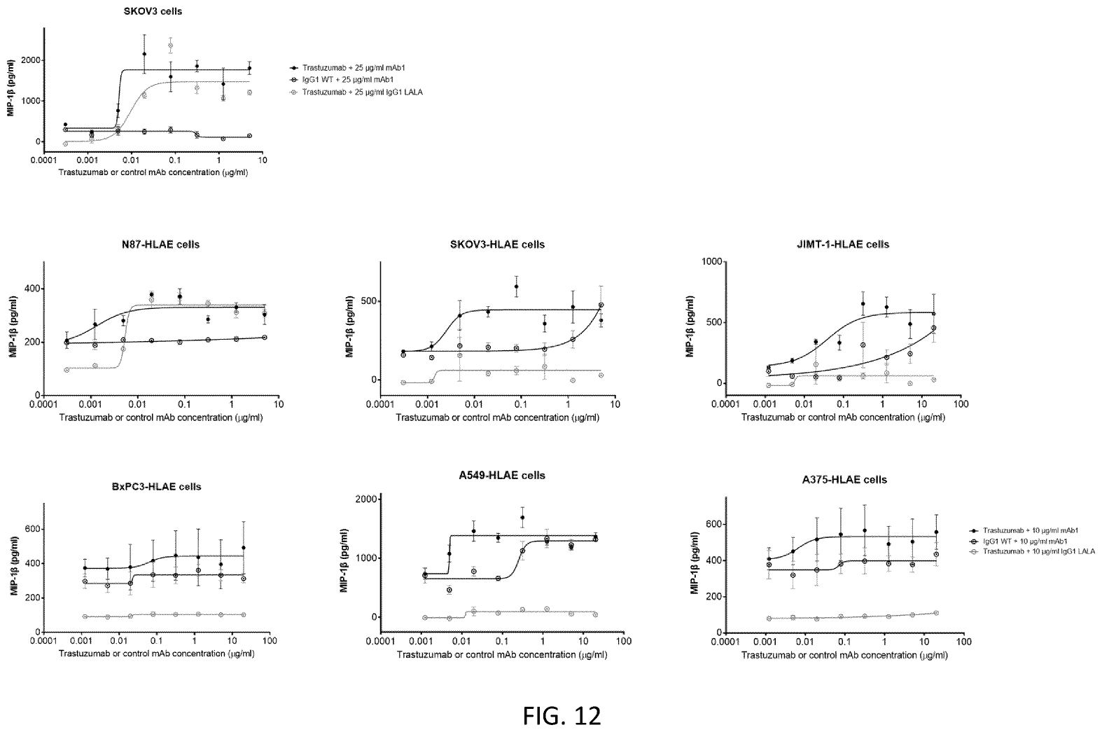

mAb1 at 25 μg/ml induced NK-cell secretion of MIP-1β when cocultured with HLA-E+ cancer cells. Addition of titrated trastuzumab to IgG1 LALA (isotype control) antibody induced a dose-dependent induction of MIP-1β secretion and this effect was increased when combining titrated trastuzumab with mAb1 (FIG. 12A). For all six cell lines transduced with HLA-E there is trastuzumab dose-dependent increase in MIP-13 when combining trastuzumab with mAb1, even at low concentrations, compared with the control antibody IgG1 LALA (FIG. 12B).

### Example 13: Combinatorial Effect of Double PD-1 and NKG2A Blockade on Peripheral Blood Lymphocytes

### Materials and Methods

Peripheral blood lymphocytes (PBLs) from three healthy donors were isolated by depleting CD14+ cells from peripheral blood mononuclear cells. 50,000 PBLs were plated in round-bottom 96-well plates with 5,000 SKOV3 cells transduced with lentiviral vectors overexpressing HLA-E and PD-L1 and activated with trastuzumab 1 μg/ml or trastuzumab 1 μg/ml plus zoledronate 1 μM. In addition, PBLs were also treated with mAb1 (10 μg/ml), pembrolizumab (10 μg/ml), the combination of mAb1 (10 μg/ml) and pembrolizumab (10 μg/ml) or with the respective isotype controls IgG1-LALA (10 μg/ml), IgG4 (10 μg/ml) or the combination of both. Supernatants were collected after 3 days of incubation and the concentrations of soluble mediators were determined by electrochemoluminescence with a multi-cytokine panel U-Plex kit (K151AEL-4, Mesoscale Discovery) on a Meso QuickPlex SQ120 plate reader (Mesoscale Discovery).

### Results

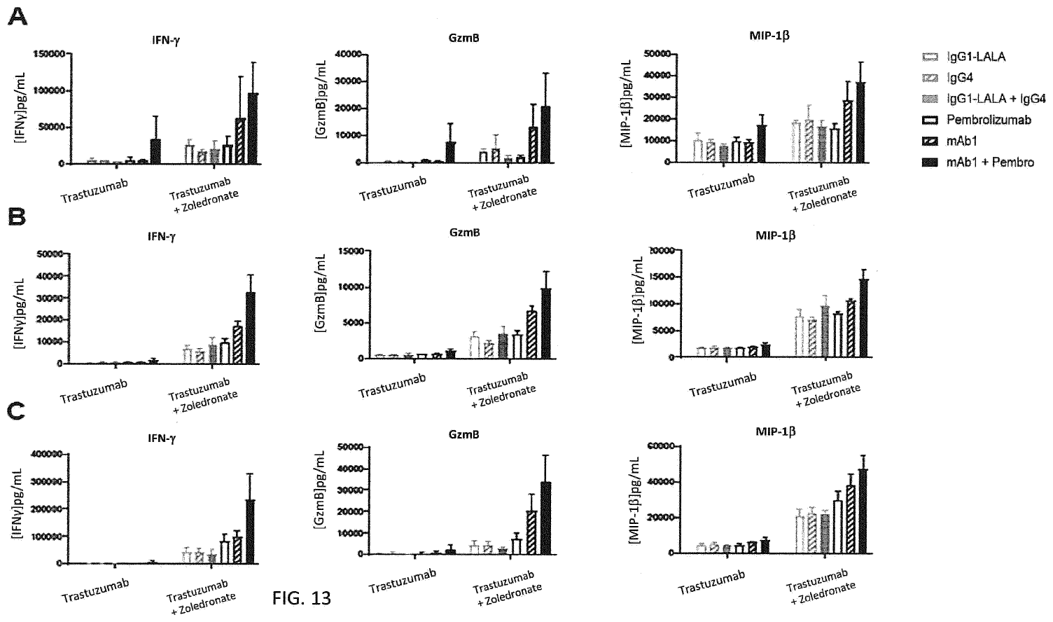

PBLs were activated by trastuzumab alone, in presence of the HER2-positive SKOV3 cells engineered to overexpress HLA-E and PD-L1, or by trastuzumab and phospho-antigens upregulated on the surface of SKOV3 cells by the addition of zoledronate. The effects of PD-1 and NKG2A checkpoint blockade were assessed individually or in combination in both activation contexts by measuring the secretion of IFN-γ, a cytokine with multiple anti-tumour activities; granzyme B, which mediates direct cytotoxicity; and MIP-1β/CCL4, a pro-inflammatory chemokine. Trastuzumab activation, alone or combined with mAb1 or pembrolizumab single agents, elicited little or no secretion of soluble mediators. However, in the case of Donor 1 the triple combination of trastuzumab, mAb1 and pembrolizumab resulted in a dramatic increase in the secretion of IFN-γ, granzyme B and MIP-1β FIG. 13A. When PBLs were activated by both trastuzumab and zoledronate-induced phospho-antigens single NKG2A blockade by mAb1 generally resulted in increased secretion of soluble mediators whereas single PD-1 blockade had no, or little effects compared to the corresponding single isotype controls. In addition, the combination of both mAb1 and pembrolizumab resulted in an increase in IFN-γ, granzyme B and MIP-13 secretion in all donors in comparison with mAb1 single treatments and compared to the combination of IgG1-LALA and IgG4 isotype controls. These results show a combinatorial effect of double NKG2A/PD-1 blockade in the context of trastuzumab stimulation.

### Example 14: Phase 1b/2 Clinical Protocol for mAb1 Combination Therapies

This example describes an open label, non-randomized, multi-arm, multi-centre, phase 1 b/2 trial investigating the safety, tolerability, and antineoplastic activity of mAb1 in combination with pembrolizumab in MSI-H/dMMR locally advanced unresectable or metastatic colorectal cancer (mCRC) and the triple combination of mAb1, pembrolizumab and trastuzumab in HER2 positive locally advanced unresectable or metastatic gastroesophageal junction (GEJ) and gastric adenocarcinoma (GA).

Maximum study duration for each participating patient: each patient will participate in the study will have approximately four months of treatment plus three months safety follow-up. For all patients, the maximum duration of the treatment period will not exceed 2 years.

### Study Design

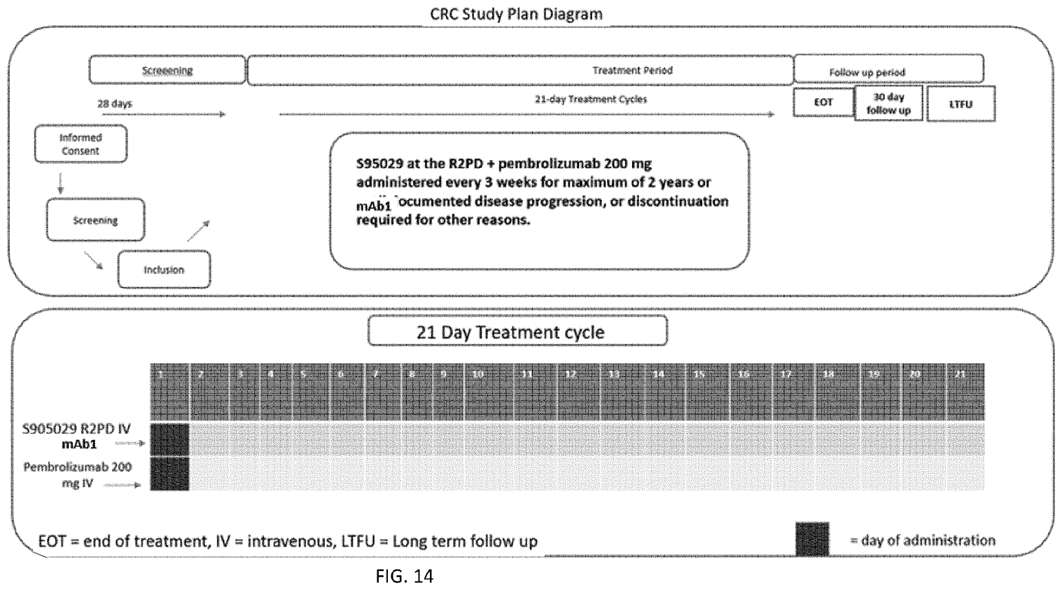

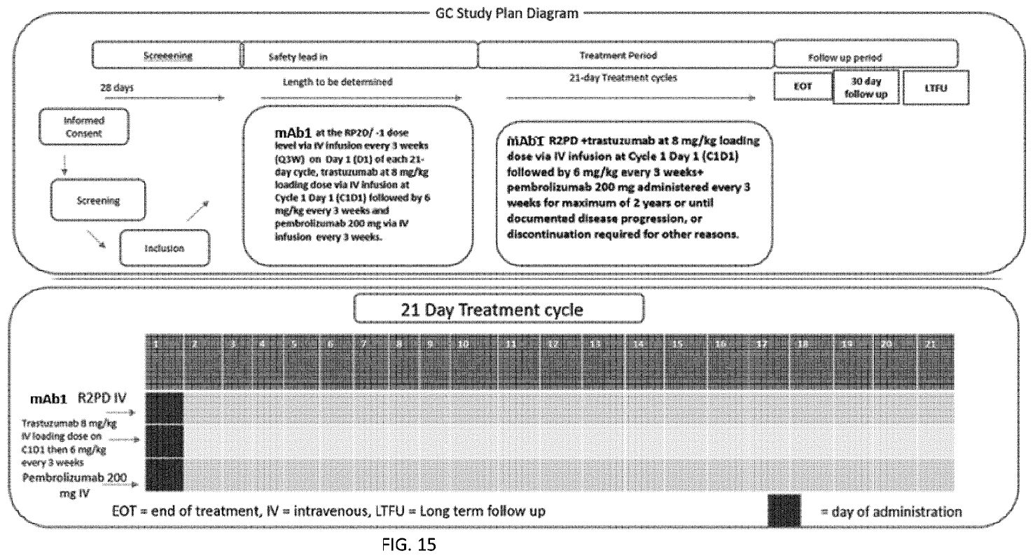

Each of the two arms mAb1+ pembrolizumab (FIG. 14) and mAb1+ pembrolizumab+trastuzumab (FIG. 15) of the clinical study is described below.

A first primary objective is to assess the antitumor activity and efficacy of the combination of mAb1 and pembrolizumab in patients with MSI-H/dMMR (deficient MisMatch Repair) colorectal cancer (CRC) and the preliminary efficacy of the combination of mAb1, pembrolizumab and trastuzumab in patients with HER2-positive gastroesophageal junction (GEJ) and gastric adenocarcinoma (GA) by assessing overall response rates (ORRs) per central assessments using Response Evaluation Criteria in Solid Tumors (RECIST) v1.1.

A second primary objective is to assess the safety and tolerability profile of the combination of mAb1 and pembrolizumab in patients with MSI-H/dMMR CRC and the safety and tolerability of the triplet combination of S095029, pembrolizumab and trastuzumab in patients with HER2-positive gastroesophageal junction (GEJ) and gastric adenocarcinoma (GA).

A first secondary objective is to characterize the pharmacokinetic (PK) profile of mAb1 in combination with pembrolizumab in patients with CRC, and the PK profile of mAb1 in combination with pembrolizumab and trastuzumab in patients with HER2-positive.

A second secondary objective is to investigate a potential PK/PD interaction between mAb1, pembrolizumab and trastuzumab in gastroesophageal and gastric cancer and between mAb1 and pembrolizumab in colorectal cancer.

A third secondary objective is to continue the assessment of the PK/PD profile to further characterize the recommended Phase 2 dose (RP2D) of each combination.

A fourth secondary objective is to evaluate the immunogenicity of each antibody in the combinations by the assessment of potential for anti-drug antibody (ADA) formation.

A fifth secondary objective is to evaluate additional efficacy parameters to assess antitumor activity of each combination.

### Methodology

This study determines the safety, tolerability and antineoplastic activity of mAb1 in combination with pembrolizumab in MSI-H/dMMR locally advanced unresectable or mCRC and the triple combination of mAb1, pembrolizumab and trastuzumab in HER2 positive locally advanced unresectable gastroesophageal cancer and gastric cancer.

### Mab1+Pembrolizumab

For the expansion phase 2 cohort, patients will be treated with S095029 at the RP2D via IV infusion every 3 weeks on Day 1 of each 21-day cycle and pembrolizumab 200 mg via IV infusion every 3 weeks on Day 1 of each 21-day cycle.

### Screening Criteria

The patients are male or female patient aged 18 years of age. The medical and therapeutic criteria include:
patients must have histologically or cytologically confirmed adenocarcinoma of locally advanced unresectable or mCRC;
patients must have a Microsatellite instability high status (MSI-H) or deficient DNA mismatch repair (dMMR) at diagnosis as per institutional guidelines or according to the College of American Pathologists;
patients must have progressed on at least 2 previous lines including an approved anti PD (L)-1 agent like pembrolizumab or nivolumab in combination with ipilimumab and standard of care chemotherapy regimens based on fluoropyrimidine with oxaliplatin or irinotecan agents;
patients with BRAF (V600E) mutation can be included and could have been treated with BRAF inhibitor, encorafenib in second line;
patients with measurable disease according to RECIST v1.1;
patients must provide an appropriate archival or a newly collected tumor biopsy;
estimated life expectancy 12 weeks;
ECOG performance status 0 or 1.
   The inclusion criteria include:
adequate haematological function based on the last assessment performed within 7 days prior to the first IMP administration, defined as: ANC 1.5×10⁹/L, haemoglobin 8 g/dL. In case of blood transfusion, platelet count 75×10⁹/L, adequate coagulation function for all patients;
adequate renal function based on the last assessment defined as creatinine clearance 30 mL/min.;
adequate hepatic function, defined as: total serum bilirubin <1.5×ULN (unless Gilbert disease confirmed <3×ULN), AST and ALT s 3.0×ULN;
serum potassium, serum phosphates, serum magnesium within normal limits with or without supplementation based on the last assessment.
   Exclusion criteria include:
major surgery within 4 weeks prior to the first IMP administration;
patients with any other serious/active/uncontrolled infection;
active hepatitis B virus infection;
carriers of HIV antibodies;
patients with a history of organ transplantation;
patients with active thrombosis, or a history of deep vein thrombosis or pulmonary embolism;
patients with active uncontrolled bleeding;
patients with non-healing wounds on any part of the body;
patients who have received anti-NKG2A mAb in the past;
patients with known, untreated central nervous system (CNS);
treatment with systemic immunosuppressive therapy;
prior radiotherapy if completed less than 4 weeks before first IMP administration;
active autoimmune disease that might deteriorate when receiving an immunostimulatory agents;
administration of a live vaccine within 28 days prior to inclusion;
known prior severe hypersensitivity to investigational products or any component in their formulations;
any contraindication present in the pembrolizumab prescribing information.
   mAb1+ pembrolizumab+trastuzumab

Patients in the lead in cohort will be treated with mAb1 at the recommended Phase 2 dose (RP2D) via IV infusion every 3 weeks on Day 1 of each 21-day cycle, trastuzumab at 8 mg/kg loading dose via IV infusion at Cycle 1 Day 1 followed by 6 mg/kg every 3 weeks and pembrolizumab 200 mg via IV infusion every 3 weeks. For the expansion phase 2 cohort, patients will be treated with mAb1 at the RP2D via IV infusion every 3 weeks on Day 1 of each 21-day cycle, trastuzumab at 8 mg/kg loading dose via IV infusion at Cycle 1 Day 1 followed by 6 mg/kg every 3 weeks and pembrolizumab 200 mg via IV infusion every 3 weeks.

### Screening Criteria

The patients are male or female patient aged 18 years of age. The medical and therapeutic criteria include:
patients with locally advanced unresectable or metastatic gastro-oesophageal cancer and gastric cancer;
patients having histologically proven unresectable locally advanced or metastatic HER2+ cancer;
patients having progressed on two previous lines of treatment including first line with fluoropyrimidine and platinum-based combinations including trastuzumab with or without pembrolizumab and a second line standard of care regimen;
patients with measurable disease according to RECIST v1.1;
Estimated life expectancy 12 weeks;
ECOG performance status 0 or 1.
   The inclusion criteria include:
adequate haematological function based on the last assessment performed within 7 days prior to the first investigational medicinal products (IMP) administration, defined as: absolute neutrophil count (ANC)≥1.5×10⁹/L, haemoglobin ≥8 g/dL, platelet count ≥75×10⁹/L;
adequate coagulation function for all patients;
adequate renal function based on the last assessment performed within 7 days prior to the first IMP administration defined as: creatinine clearance 30 mL/min.;
adequate hepatic function based on the last assessment performed within 7 days prior to the first IMP administration, defined as: total serum bilirubin <1.5×upper limit of normal (ULN); aspartate aminotransferase (AST) and alanine aminotransferase (ALT)≤3×ULN.
   Exclusion criteria include:
left ventricle ejection fraction (LVEF)<50% by echocardiogram;
any contraindication present in the trastuzumab or pembrolizumab;
major surgery within 4 weeks prior to the first IMP administration;
patients with any other serious/active/uncontrolled infection;
active hepatitis B virus infection;
carriers of HIV antibodies;
patients with a history of organ transplantation;
patients with active thrombosis, or a history of deep vein thrombosis or pulmonary embolism;
patients with active uncontrolled bleeding;
patients with a known clinically significant cardiovascular disease;
history of gastrointestinal perforation, or intra-abdominal abscess;
history of cirrhosis or chronic liver condition;
history of pulmonary fibrosis or relevant uncontrolled chronic pulmonary condition;
patients with non-healing wounds on any part of the body;
patients who have received anti-NKG2A mAb in the past;
patients with known, untreated central nervous system (CNS);
treatment with systemic immunosuppressive therapy;
prior radiotherapy if completed less than 4 weeks before first IMP administration;
active autoimmune disease.

### Criteria for Evaluation

For both arms, tumor assessment will be performed every 9 weeks (+/−7 days) by medical imaging and in case of response, confirmation will be required at least 4 weeks after. Patients will be followed every 90 days (+/−7 days) for assessment of survival status after disease progression. The survival follow-up period will last up to 2 years.

TABLE 4
Sequences

| | | | |
|---|---|---|---|
| Antibody | SEQ | Description | Sequence |

NKG2A

| | | | |
|---|---|---|---|
| mAb1 | 1 | H-CDR1 | GFTFSDYY |
| | 2 | H-CDR2 | ISTSGSTI |
| | 3 | H-CDR3 | CARDHYYSRGVIGYW |
| | 4 | L-CDR1 | QGISSW |
| | 5 | L-CDR2 | AAS |
| | 6 | L-CDR3 | CQQANSFPYTF |
| | 7 | VH | QVQLVESGGGLVKPGGSLRLSCAASGFTFSDYYMRWIRQAPGKGLEWVSHISTSGSTIYYADSVKGRFT |
| | | | ISRDNAKNSLYLQMNSLRAEDTAVYYCARDHYYSRGVIGYWGQGTLVTVSS |
| | 8 | VL | DIQMTQSPSSVSASVGDRVTITCRASQGISSWLAWYQQKPGKAPKLLIYAASSLQSGVPSRFSGSGSGT |
| | | | DFTLTISSLQPEDFATYYCQQANSFPYTFGQGTKLEIK |
| | 9 | HC | QVQLVESGGGLVKPGGSLRLSCAASGFTFSDYYMRWIRQAPGKGLEWVSHISTSGSTIYYADSVKGRFT |
| | | | ISRDNAKNSLYLQMNSLRAEDTAVYYCARDHYYSRGVIGYWGQGTLVTVSSASTKGPSVFPLAPSSKST |
| | | | SGGTAALGCLVKDYFPEPVTVSWNSGALTSGVHTFPAVLQSSGLYSLSSVVTVPSSSLGTQTYICNVNH |
| | | | KPSNTKVDKRVEPKSCDKTHTCPPCPAPEAAGGPSVFLFPPKPKDTLMISRTPEVTCVVVDVSHEDPEV |
| | | | KFNWYVDGVEVHNAKTKPREEQYNSTYRVVSVLTVLHQDWLNGKEYKCKVSNKALPAPIEKTISKAKGQ |
| | | | PREPQVYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| | 10 | LC | DIQMTQSPSSVSASVGDRVTITCRASQGISSWLAWYQQKPGKAPKLLIYAASSLQSGVPSRFSGSGSGT |
| | | | DFTLTISSLQPEDFATYYCQQANSFPYTFGQGTKLEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| mAb2 | 11 | H-CDR1 | GFTFSSYA |
| | 12 | H-CDR2 | ISNSGGTT |
| | 13 | H-CDR3 | CAKAHYYARGYFDYW |
| | 14 | L-CDR1 | QGISSW |
| | 15 | L-CDR2 | AAS |
| | 16 | L-CDR3 | CQQANSFPYTF |
| | 17 | VH | EVQLVESGGGLVQPGGSLRLSCAASGFTFSSYAMNWVRQAPGKGLEWVSTISNSGGTTYYADSVKGRFT |
| | | | ISRDNSKDTLYLQMNSLRAEDTAVYYCAKAHYYARGYFDYWGQGTLVTVSS |
| | 18 | VL | DIQMTQSPSSVSASVGDRVTITCRASQGISSWLAWYQQKSGKAPKLLIYAASSLQIGVPSRFSGSGSGT |
| | | | DFTLTISSLQPEDFATYYCQQANSFPYTFGQGTKLEIK |
| | 19 | HC | EVQLVESGGGLVQPGGSLRLSCAASGFTFSSYAMNWVRQAPGKGLEWVSTISNSGGTTYYADSVKGRFT |
| | | | ISRDNSKDTLYLQMNSLRAEDTAVYYCAKAHYYARGYFDYWGQGTLVTVSSASTKGPSVFPLAPSSKST |
| | | | SGGTAALGCLVKDYFPEPVTVSWNSGALTSGVHTFPAVLQSSGLYSLSSVVTVPSSSLGTQTYICNVNH |
| | | | KPSNTKVDKRVEPKSCDKTHTCPPCPAPEAAGGPSVFLFPPKPKDTLMISRTPEVTCVVVDVSHEDPEV |
| | | | KFNWYVDGVEVHNAKTKPREEQYNSTYRVVSVLTVLHQDWLNGKEYKCKVSNKALPAPIEKTISKAKGQ |
| | | | PREPQVYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| | 20 | LC | DIQMTQSPSSVSASVGDRVTITCRASQGISSWLAWYQQKSGKAPKLLIYAASSLQIGVPSRFSGSGSGT |
| | | | DFTLTISSLQPEDFATYYCQQANSFPYTFGQGTKLEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |

PD-1

| | | | |
|---|---|---|---|
| nivo- | 21 | H-CDR1 | NSGMH |
| lumab | 22 | H-CDR2 | VIWYDGSKRYYADSVKG |
| | 23 | H-CDR3 | NDDY |
| | 24 | L-CDR1 | RASQSVSSYLA |
| | 25 | L-CDR2 | DASNRAT |
| | 26 | L-CDR3 | QQSSNWPRT |
| | 27 | VH | QVQLVESGGGVVQPGRSLRLDCKASGITFSNSGMHWVRQAPGKGLEWVAVIWYDGSKRYYADSVKGRFT |
| | | | ISRDNSKNTLFLQMNSLRAEDTAVYYCATNDDYWGQGTLVTVSS |
| | 28 | VL | EIVLTQSPATLSLSPGERATLSCRASQSVSSYLAWYQQKPGQAPRLLIYDASNRATGIPARFSGSGSGT |
| | | | DFTLTISSLEPEDFAVYYCQQSSNWPRTFGQGTKVEIK |
| | 29 | HC | QVQLVESGGGVVQPGRSLRLDCKASGITFSNSGMHWVRQAPGKGLEWVAVIWYDGSKRYYADSVKGRFT |
| | | | ISRDNSKNTLFLQMNSLRAEDTAVYYCATNDDYWGQGTLVTVSSASTKGPSVFPLAPCSRSTSESTAAL |
| | | | GCLVKDYFPEPVTVSWNSGALTSGVHTFPAVLQSSGLYSLSSVVTVPSSSLGTKTYTCNVDHKPSNTKV |
| | | | DKRVESKYGPPCPPCPAPEFLGGPSVFLFPPKPKDTLMISRTPEVTCVVVDVSQEDPEVQFNWYVDGVE |
| | | | VHNAKTKPREEQFNSTYRVVSVLTVLHQDWLNGKEYKCKVSNKGLPSSIEKTISKAKGQPREPQVYTLP |
| | | | PSQEEMTKNQVSLTCLVKGFYPSDIAVEWESNGQPENNYKTTPPVLDSDGSFFLYSRLTVDKSRWQEGN |
| | | | VFSCSVMHEALHNHYTQKSLSLSLGK |
| | 30 | LC | EIVLTQSPATLSLSPGERATLSCRASQSVSSYLAWYQQKPGQAPRLLIYDASNRATGIPARFSGSGSGT |
| | | | DFTLTISSLEPEDFAVYYCQQSSNWPRTFGQGTKVEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| pembro- | 31 | H-CDR1 | NYYMY |
| lizumab | 32 | H-CDR2 | GINPSNGGTNFNEKFKN |
| | 33 | H-CDR3 | RDYRFDMGFDY |
| | 34 | L-CDR1 | RASKGVSTSGYSYLH |
| | 35 | L-CDR2 | LASYLES |
| | 36 | L-CDR3 | QHSRDLPLT |
| | 37 | VH | QVQLVQSGVEVKKPGASVKVSCKASGYTFTNYYMYWVRQAPGQGLEWMGGINPSNGGTNFNEKFKNRVT |
| | | | LTTDSSTTTAYMELKSLQFDDTAVYYCARRDYRFDMGFDYWGQGTTVTVSS |
| | 38 | VL | EIVLTQSPATLSLSPGERATLSCRASKGVSTSGYSYLHWYQQKPGQAPRLLIYLASYLESGVPARFSGS |
| | | | GSGTDFTLTISSLEPEDFAVYYCQHSRDLPLTFGGGTKVEIK |
| | 39 | HC | QVQLVQSGVEVKKPGASVKVSCKASGYTFTNYYMYWVRQAPGQGLEWMGGINPSNGGTNFNEKFKNRVT |
| | | | LTTDSSTTTAYMELKSLQFDDTAVYYCARRDYRFDMGFDYWGQGTTVTVSSASTKGPSVFPLAPCSRST |
| | | | SESTAALGCLVKDYFPEPVTVSWNSGALTSGVHTFPAVLQSSGLYSLSSVVTVPSSSLGTKTYTCNVDH |
| | | | KPSNTKVDKRVESKYGPPCPPCPAPEFLGGPSVFLFPPKPKDTLMISRTPEVTCVVVDVSQEDPEVQFN |
| | | | WYVDGVEVHNAKTKPREEQFNSTYRVVSVLTVLHQDWLNGKEYKCKVSNKGLPSSIEKTISKAKGQPRE |
| | | | PQVYTLPPSQEEMTKNQVSLTCLVKGFYPSDIAVEWESNGQPENNYKTTPPVLDSDGSFFLYSRLTVDK |
| | | | SRWQEGNVFSCSVMHEALHNHYTQKSLSLSLGK |
| | 40 | LC | EIVLTQSPATLSLSPGERATLSCRASKGVSTSGYSYLHWYQQKPGQAPRLLIYLASYLESGVPARFSGS |
| | | | GSGTDFTLTISSLEPEDFAVYYCQHSRDLPLTFGGGTKVEIKRTVAAPSVFIFPPSDEQLKSGTASVVC |
| | | | LLNNFYPREAKVQWKVDNALQSGNSQESVTEQDSKDSTYSLSSTLTLSKADYEKHKVYACEVTHQGLSS |
| | | | PVTKSFNRGEC |
| cemi- | 41 | H-CDR1 | GFTFSNFG |
| plimab | 42 | H-CDR2 | ISGGGRDT |
| | 43 | H-CDR3 | VKWGNIYFDY |
| | 44 | L-CDR1 | LSINTF |
| | 45 | L-CDR2 | AAS |
| | 46 | L-CDR3 | QQSSNTPFT |
| | 47 | VH | EVQLLESGGVLVQPGGSLRLSCAASGFTFSNFGMTWVRQAPGKGLEWVSGISGGGRDTYFADSVKGRFT |
| | | | ISRDNSKNTLYLQMNSLKGEDTAVYYCVKWGNIYFDYWGQGTLVTVSS |
| | 48 | VL | DIQMTQSPSSLSASVGDSITITCRASLSINTFLNWYQQKPGKAPNLLIYAASSLHGGVPSRFSGSGSGT |
| | | | DFTLTIRTLQPEDFATYYCQQSSNTPFTFGPGTVVDER |
| | 49 | HC | EVQLLESGGVLVQPGGSLRLSCAASGFTFSNFGMTWVRQAPGKGLEWVSGISGGGRDTYFADSVKGRFT |
| | | | ISRDNSKNTLYLQMNSLKGEDTAVYYCVKWGNIYFDYWGQGTLVTVSSASTKGPSVFPLAPCSRSTSES |
| | | | TAALGCLVKDYFPEPVTVSWNSGALTSGVHTFPAVLQSSGLYSLSSVVTVPSSSLGTKTYTCNVDHKPS |
| | | | NTKVDKRVESKYGPPCPPCPAPEFLGGPSVFLFPPKPKDTLMISRTPEVTCVVVDVSQEDPEVQFNWYV |
| | | | DGVEVHNAKTKPREEQFNSTYRVVSVLTVLHQDWLNGKEYKCKVSNKGLPSSIEKTISKAKGQPREPQV |
| | | | YTLPPSQEEMTKNQVSLTCLVKGFYPSDIAVEWESNGQPENNYKTTPPVLDSDGSFFLYSRLTVDKSRW |
| | | | QEGNVFSCSVMHEALHNHYTQKSLSLSLGK |
| | 50 | LC | DIQMTQSPSSLSASVGDSITITCRASLSINTELNWYQQKPGKAPNLLIYAASSLHGGVPSRFSGSGSGT |
| | | | DFTLTIRTLQPEDFATYYCQQSSNTPFTFGPGTVVDFRRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| dosta- | 51 | H-CDR1 | GFTFSSYDMS |
| rlimab | 52 | H-CDR2 | TISGGGSYTY |
| | 53 | H-CDR3 | PYYAMDY |
| | 54 | L-CDR1 | KASQDVGTAVA |
| | 55 | L-CDR2 | WASTLHT |
| | 56 | L-CDR3 | QHYSSYPWT |
| | 57 | VH | EVQLLESGGGLVQPGGSLRLSCAASGFTFSSYDMSWVRQAPGKGLEWVSTISGGGSYTYYQDSVKGRFT |
| | | | ISRDNSKNTLYLQMNSLRAEDTAVYYCASPYYAMDYWGQGTTVTVSS |
| | 58 | VL | DIQLTQSPSFLSAYVGDRVTITCKASQDVGTAVAWYQQKPGKAPKLLIYWASTLHTGVPSRFSGSGSGT |
| | | | EFTLTISSLQPEDFATYYCQHYSSYPWTFGQGTKLEIK |
| | 59 | HC | EVQLLESGGGLVQPGGSLRLSCAASGFTFSSYDMSWVRQAPGKGLEWVSTISGGGSYTYYQDSVKGRFT |
| | | | ISRDNSKNTLYLQMNSLRAEDTAVYYCASPYYAMDYWGQGTTVTVSSASTKGPSVFPLAPCSRSTSEST |
| | | | AALGCLVKDYFPEPVTVSWNSGALTSGVHTFPAVLQSSGLYSLSSVVTVPSSSLGTKTYTCNVDHKPSN |
| | | | TKVDKRVESKYGPPCPPCPAPEFLGGPSVFLFPPKPKDTLMISRTPEVTCVVVDVSQEDPEVQFNWYVD |
| | | | GVEVHNAKTKPREEQFNSTYRVVSVLTVLHQDWLNGKEYKCKVSNKGLPSSIEKTISKAKGQPREPQVY |
| | | | TLPPSQEEMTKNQVSLTCLVKGFYPSDIAVEWESNGQPENNYKTTPPVLDSDGSFFLYSRLTVDKSRWQ |
| | | | EGNVFSCSVMHEALHNHYTQKSLSLSLGK |
| | 60 | LC | DIQLTQSPSFLSAYVGDRVTITCKASQDVGTAVAWYQQKPGKAPKLLIYWASTLHTGVPSRFSGSGSGT |
| | | | EFTLTISSLQPEDFATYYCQHYSSYPWTFGQGTKLEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| mAb3 | 61 | H-CDR1 | GFTFTRYD |
| | 62 | H-CDR2 | IGDSNKMT |
| | 63 | H-CDR3 | CAKGSCIACWDEAGRIDAW |
| | 64 | L-CDR1 | GSYDGSSY |
| | 65 | L-CDR2 | NNN |
| | 66 | L-CDR3 | CGSYDRPETNSDYVGMF |
| | 67 | VH | EVQLLESGGGLVQPGGSLRLSCAASGFTFTRYDMVWVRQAPGKGLEWVAGIGDSNKMTRYAPAVKGRAT |
| | | | ISRDNSKNTLYLQMNSLRAEDTAVYYCAKGSCIACWDEAGRIDAWGQGTLVTVSS |
| | 68 | VL | SYELTQDPAVSVALGQTVRITCSGGGSYDGSSYYGWYQQKPGQAPVTVIYNNNNRPSDIPDRFSGSSSG |
| | | | NTASLTITGAQAEDEADYYCGSYDRPETNSDYVGMFGSGTKVTVL |
| | 69 | HC | EVQLLESGGGLVQPGGSLRLSCAASGFTFTRYDMVWVRQAPGKGLEWVAGIGDSNKMTRYAPAVKGRAT |
| | | | ISRDNSKNTLYLQMNSLRAEDTAVYYCAKGSCIACWDEAGRIDAWGQGTLVTVSSASTKGPSVFPLAPS |
| | | | SKSTSGGTAALGCLVKDYFPEPVTVSWNSGALTSGVHTFPAVLQSSGLYSLSSVVTVPSSSLGTQTYIC |
| | | | NVNHKPSNTKVDKRVEPKSCDKTHTCPPCPAPEAAGGPSVFLFPPKPKDTLMISRTPEVTCVVVDVSHE |
| | | | DPEVKFNWYVDGVEVHNAKTKPREEQYNSTYRVVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| | 70 | LC | SYELTQDPAVSVALGQTVRITCSGGGSYDGSSYYGWYQQKPGQAPVTVIYNNNNRPSDIPDRFSGSSSG |
| | | | NTASLTITGAQAEDEADYYCGSYDRPETNSDYVGMFGSGTKVTVLGQPKANPTVTLFPPSSEELQANKA |
| | | | TLVCLISDFYPGAVTVAWKADGSPVKAGVETTKPSKQSNNKYAASSYLSLTPEQWKSHRSYSCQVTHEG |
| | | | STVEKTVAPTECS |
| reti- | 71 | H-CDR1 | SYWMN |
| fanlimab | 72 | H-CDR2 | VIHPSDSETWLDQKFKD |
| | 73 | H-CDR3 | EHYGTSPFAY |
| | 74 | L-CDR1 | RASESVDNYGMSFMN |
| | 75 | L-CDR2 | AASNQGS |
| | 76 | L-CDR3 | QQSKEVPYT |
| | 77 | VH | QVQLVQSGAEVKKPGASVKVSCKASGYSFTSYWMNWVRQAPGQGLEWIGVIHPSDSETWLDQKFKDRVT |
| | | | ITVDKSTSTAYMELSSLRSEDTAVYYCAREHYGTSPFAYWGQGTLVTVSS |
| | 78 | VL | EIVLTQSPATLSLSPGERATLSCRASESVDNYGMSFMNWFQQKPGQPPKLLIHAASNQGSGVPSRFSGS |
| | | | GSGTDFTLTISSLEPEDFAVYFCQQSKEVPYTFGGGTKVEIK |
| | 79 | HC | QVQLVQSGAEVKKPGASVKVSCKASGYSFTSYWMNWVRQAPGQGLEWIGVIHPSDSETWLDQKFKDRVT |
| | | | ITVDKSTSTAYMELSSLRSEDTAVYYCAREHYGTSPFAYWGQGTLVTVSSASTKGPSVFPLAPCSRSTS |
| | | | ESTAALGCLVKDYFPEPVTVSWNSGALTSGVHTFPAVLQSSGLYSLSSVVTVPSSSLGTKTYTCNVDHK |
| | | | PSNTKVDKRVESKYGPPCPPCPAPEFLGGPSVFLFPPKPKDTLMISRTPEVTCVVVDVSQEDPEVQFNW |
| | | | YVDGVEVHNAKTKPREEQFNSTYRVVSVLTVLHQDWLNGKEYKCKVSNKGLPSSIEKTISKAKGQPREP |
| | | | QVYTLPPSQEEMTKNQVSLTCLVKGFYPSDIAVEWESNGQPENNYKTTPPVLDSDGSFFLYSRLTVDKS |
| | | | RWQEGNVFSCSVMHEALHNHYTQKSLSLSLG |
| | 80 | LC | EIVLTQSPATLSLSPGERATLSCRASESVDNYGMSFMNWFQQKPGQPPKLLIHAASNQGSGVPSRFSGS |
| | | | GSGTDFTLTISSLEPEDFAVYFCQQSKEVPYTFGGGTKVEIKRTVAAPSVFIFPPSDEQLKSGTASVVC |
| | | | LLNNFYPREAKVQWKVDNALQSGNSQESVTEQDSKDSTYSLSSTLTLSKADYEKHKVYACEVTHQGLSS |
| | | | PVTKSFNRGEC |

PD-L1

| | | | |
|---|---|---|---|
| atezo- | 81 | H-CDR1 | GFTFSDSWIH |
| lizumab | 82 | H-CDR2 | AWISPYGGSTYYADSVKG |
| | 83 | H-CDR3 | RHWPGGFDY |
| | 84 | L-CDR1 | RASQDVSTAVA |
| | 85 | L-CDR2 | SASFLYS |
| | 86 | L-CDR3 | QQYLYHPAT |
| | 87 | VH | EVQLVESGGGLVQPGGSLRLSCAASGFTFSDSWIHWVRQAPGKGLEWVAWISPYGGSTYYADSVKGRFT |
| | | | ISADTSKNTAYLQMNSLRAEDTAVYYCARRHWPGGFDYWGQGTLVTVSS |
| | 88 | VL | DIQMTQSPSSLSASVGDRVTITCRASQDVSTAVAWYQQKPGKAPKLLIYSASFLYSGVPSRFSGSGSGT |
| | | | DFTLTISSLQPEDFATYYCQQYLYHPATFGQGTKVEIK |
| | 89 | HC | EVQLVESGGGLVQPGGSLRLSCAASGFTFSDSWIHWVRQAPGKGLEWVAWISPYGGSTYYADSVKGRFT |
| | | | ISADTSKNTAYLQMNSLRAEDTAVYYCARRHWPGGFDYWGQGTLVTVSSASTKGPSVFPLAPSSKSTSG |
| | | | GTAALGCLVKDYFPEPVTVSWNSGALTSGVHTFPAVLQSSGLYSLSSVVTVPSSSLGTQTYICNVNHKP |
| | | | SNTKVDKKVEPKSCDKTHTCPPCPAPELLGGPSVFLFPPKPKDTLMISRTPEVTCVVVDVSHEDPEVKF |
| | | | NWYVDGVEVHNAKTKPREEQYASTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGK |
| | 90 | LC | DIQMTQSPSSLSASVGDRVTITCRASQDVSTAVAWYQQKPGKAPKLLIYSASFLYSGVPSRFSGSGSGT |
| | | | DFTLTISSLQPEDFATYYCQQYLYHPATFGQGTKVEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| avelumab | 91 | H-CDR1 | SYIMM |
| | 92 | H-CDR2 | SIYPSGGITFYADTVKG |
| | 93 | H-CDR3 | IKLGTVTTVDY |
| | 94 | L-CDR1 | SCTGTSSDVGGYNYVS |
| | 95 | L-CDR2 | DVSNRPS |
| | 96 | L-CDR3 | SSYTSSSTRV |
| | 97 | VH | EVQLLESGGGLVQPGGSLRLSCAASGFTFSSYIMMWVRQAPGKGLEWVSSIYPSGGITFYADTVKGRFT |
| | | | ISRDNSKNTLYLQMNSLRAEDTAVYYCARIKLGTVTTVDYWGQGTLVTVSS |
| | 98 | VL | QSALTQPASVSGSPGQSITISCTGTSSDVGGYNYVSWYQQHPGKAPKLMIYDVSNRPSGVSNRFSGSKS |
| | | | GNTASLTISGLQAEDEADYYCSSYTSSSTRVFGTGTKVTVL |
| | 99 | HC | EVQLLESGGGLVQPGGSLRLSCAASGFTFSSYIMMWVRQAPGKGLEWVSSIYPSGGITFYADTVKGRFT |
| | | | ISRDNSKNTLYLQMNSLRAEDTAVYYCARIKLGTVTTVDYWGQGTLVTVSSASTKGPSVFPLAPSSKST |
| | | | SGGTAALGCLVKDYFPEPVTVSWNSGALTSGVHTFPAVLQSSGLYSLSSVVTVPSSSLGTQTYICNVNH |
| | | | KPSNTKVDKKVEPKSCDKTHTCPPCPAPELLGGPSVFLFPPKPKDTLMISRTPEVTCVVVDVSHEDPEV |
| | | | KFNWYVDGVEVHNAKTKPREEQYNSTYRVVSVLTVLHQDWLNGKEYKCKVSNKALPAPIEKTISKAKGQ |
| | | | PREPQVYTLPPSRDELTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| | 100 | LC | QSALTQPASVSGSPGQSITISCTGTSSDVGGYNYVSWYQQHPGKAPKLMIYDVSNRPSGVSNRFSGSKS |
| | | | GNTASLTISGLQAEDEADYYCSSYTSSSTRVFGTGTKVTVLGQPKANPTVTLFPPSSEELQANKATLVC |
| | | | LISDFYPGAVTVAWKADGSPVKAGVETTKPSKQSNNKYAASSYLSLTPEQWKSHRSYSCQVTHEGSTVE |
| | | | KTVAPTECS |
| durva- | 101 | H-CDR1 | GFTFSRYWMS |
| lumab | 102 | H-CDR2 | NIKQDGSEKYYVDSVKG |
| | 103 | H-CDR3 | EGGWFGELAFDY |
| | 104 | L-CDR1 | RASQRVSSSYLA |
| | 105 | L-CDR2 | DASSRAT |
| | 106 | L-CDR3 | QQYGSLPWT |
| | 107 | VH | EVQLVESGGGLVQPGGSLRLSCAASGFTFSRYWMSWVRQAPGKGLEWVANIKQDGSEKYYVDSVKGRFT |
| | | | ISRDNAKNSLYLQMNSLRAEDTAVYYCAREGGWFGELAFDYWGQGTLVTVSS |
| | 108 | VL | EIVLTQSPGTLSLSPGERATLSCRASQRVSSSYLAWYQQKPGQAPRLLIYDASSRATGIPDRFSGSGSG |
| | | | TDFTLTISRLEPEDFAVYYCQQYGSLPWTFGQGTKVEIK |
| | 109 | HC | EVQLVESGGGLVQPGGSLRLSCAASGFTFSRYWMSWVRQAPGKGLEWVANIKQDGSEKYYVDSVKGRFT |
| | | | ISRDNAKNSLYLQMNSLRAEDTAVYYCAREGGWFGELAFDYWGQGTLVTVSSASTKGPSVFPLAPSSKS |
| | | | TSGGTAALGCLVKDYFPEPVTVSWNSGALTSGVHTFPAVLQSSGLYSLSSVVTVPSSSLGTQTYICNVN |
| | | | HKPSNTKVDKRVEPKSCDKTHTCPPCPAPEFEGGPSVFLFPPKPKDTLMISRTPEVTCVVVDVSHEDPE |
| | | | VKFNWYVDGVEVHNAKTKPREEQYNSTYRVVSVLTVLHQDWLNGKEYKCKVSNKALPASIEKTISKAKG |
| | | | QPREPQVYTLPPSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGK |
| | 110 | LC | EIVLTQSPGTLSLSPGERATLSCRASQRVSSSYLAWYQQKPGQAPRLLIYDASSRATGIPDRFSGSGSG |
| | | | TDFTLTISRLEPEDFAVYYCQQYGSLPWTFGQGTKVEIKRTVAAPSVFIFPPSDEQLKSGTASVVCLLN |
| | | | NFYPREAKVQWKVDNALQSGNSQESVTEQDSKDSTYSLSSTLTLSKADYEKHKVYACEVTHQGLSSPVT |
| | | | KSFNRGEC |

EGFR

| | | | |
|---|---|---|---|
| cetuxi- | 111 | H-CDR1 | NYGVH |
| mab | 112 | H-CDR2 | VIWSGGNTDYNTPFTS |
| | 113 | H-CDR3 | ALTYYDYEFAY |
| | 114 | L-CDR1 | RASQSIGTNIH |
| | 115 | L-CDR2 | YASESIS |
| | 116 | L-CDR3 | QQNNNWPTT |
| | 117 | VH | QVQLKQSGPGLVQPSQSLSITCTVSGFSLTNYGVHWVRQSPGKGLEWLGVIWSGGNTDYNTPFTSRLSI |
| | | | NKDNSKSQVFFKMNSLQSNDTAIYYCARALTYYDYEFAYWGQGTLVTVSA |
| | 118 | VL | DILLTQSPVILSVSPGERVSFSCRASQSIGTNIHWYQQRTNGSPRLLIKYASESISGIPSRFSGSGSGT |
| | | | DFTLSINSVESEDIADYYCQQNNNWPTTFGAGTKLELK |
| | 119 | HC | QVQLKQSGPGLVQPSQSLSITCTVSGFSLTNYGVHWVRQSPGKGLEWLGVIWSGGNTDYNTPFTSRLSI |
| | | | NKDNSKSQVFFKMNSLQSNDTAIYYCARALTYYDYEFAYWGQGTLVTVSAASTKGPSVFPLAPSSKSTS |
| | | | GGTAALGCLVKDYFPEPVTVSWNSGALTSGVHTFPAVLQSSGLYSLSSVVTVPSSSLGTQTYICNVNHK |
| | | | PSNTKVDKKVEPKSCDKTHTCPPCPAPELLGGPSVFLFPPKPKDTLMISRTPEVTCVVVDVSHEDPEVK |
| | | | FNWYVDGVEVHNAKTKPREEQYNSTYRVVSVLTVLHQDWLNGKEYKCKVSNKALPAPIEKTISKAKGQP |
| | | | REPQVYTLPPSRDELTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGK |
| | 120 | LC | DILLTQSPVILSVSPGERVSFSCRASQSIGTNIHWYQQRINGSPRLLIKYASESISGIPSRFSGSGSGT |
| | | | DFTLSINSVESEDIADYYCQQNNNWPTTFGAGTKLELKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| pani- | 121 | H-CDR1 | SGDYYWTW |
| tumumab | 122 | H-CDR2 | HIYYSGNTNYNPSLKSR |
| | 123 | H-CDR3 | DRVTGAFDI |
| | 124 | L-CDR1 | ASQDISNYLN |
| | 125 | L-CDR2 | DASNLET |
| | 126 | L-CDR3 | QHFDHLPLA |
| | 127 | VH | QVQLQESGPGLVKPSETLSLTCTVSGGSVSSGDYYWTWIRQSPGKGLEWIGHIYYSGNTNYNPSLKSRL |
| | | | TISIDTSKTQFSLKLSSVTAADTAIYYCVRDRVTGAFDIWGQGTMVTVSS |
| | 128 | VL | DIQMTQSPSSLSASVGDRVTITCQASQDISNYLNWYQQKPGKAPKLLIYDASNLETGVPSRFSGSGSGT |
| | | | DFTFTISSLQPEDIATYFCQHFDHLPLAFGGGTKVEIK |
| | 129 | HC | QVQLQESGPGLVKPSETLSLTCTVSGGSVSSGDYYWTWIRQSPGKGLEWIGHIYYSGNTNYNPSLKSRL |
| | | | TISIDTSKTQFSLKLSSVTAADTAIYYCVRDRVTGAFDIWGQGTMVTVSSASTKGPSVFPLAPCSRSTS |
| | | | ESTAALGCLVKDYFPEPVTVSWNSGALTSGVHTFPAVLQSSGLYSLSSVVTVPSSNFGTQTYTCNVDHK |
| | | | PSNTKVDKTVERKCCVECPPCPAPPVAGPSVFLFPPKPKDTLMISRTPEVTCVVVDVSHEDPEVQfNWY |
| | | | VDGVEVHNAKTKPREEQFNSTFRVVSVLTVVHQDWLNGKEYKCKVSNKGLPAPIEKTISKTKGQPREPQ |
| | | | VYTLPPSREEMTKNQVSLTCLVKGFYPSDIAVEWESNGQPENNYKTTPPMLDSDGSFFLYSKLTVDKSR |
| | | | WQQGNVFSCSVMHEALHNHYTQKSLSLSPGK |
| | 130 | LC | DIQMTQSPSSLSASVGDRVTITCQASQDISNYLNWYQQKPGKAPKLLIYDASNLETGVPSRFSGSGSGT |
| | | | DFTFTISSLQPEDIATYFCQHFDHLPLAFGGGTKVEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| futuxi- | 131 | H-CDR1 | GYTFTSYW |
| mab | 132 | H-CDR2 | IYPGSRST |
| | 133 | H-CDR3 | TRNGDYYVSSGDAMDY |
| | 134 | L-CDR1 | QDIGNY |
| | 135 | L-CDR2 | YTS |
| | 136 | L-CDR3 | QHYNTVPPT |
| | 137 | VH | EVQLQQPGSELVRPGASVKLSCKASGYTFTSYWMHWVKQRPGQGLEWIGNIYPGSRSTNYDEKFKSKAT |
| | | | LTVDTSSSTAYMQLSSLTSEDSAVYYCTRNGDYYVSSGDAMDYWGQGTSVTVSS |
| | 138 | VL | DIQMTQTTSSLSASLGDRVTISCRTSQDIGNYLNWYQQKPDGTVKLLIYYTSRLHSGVPSRFSGSGSGT |
| | | | DFSLTINNVEQEDVATYFCQHYNTVPPTFGGGTKLEIK |
| | 139 | HC | EVQLQQPGSELVRPGASVKLSCKASGYTFTSYWMHWVKQRPGQGLEWIGNIYPGSRSTNYDEKFKSKAT |
| | | | LTVDTSSSTAYMQLSSLTSEDSAVYYCTRNGDYYVSSGDAMDYWGQGTSVTVSSASTKGPSVFPLAPSS |
| | | | KSTSGGTAALGCLVKDYFPEPVTVSWNSGALTSGVHTFPAVLQSSGLYSLSSVVTVPSSSLGTQTYICN |
| | | | VNHKPSNTKVDKRVEPKSCDKTHTCPPCPAPELLGGPSVFLFPPKPKDTLMISRTPEVTCVVVDVSHED |
| | | | PEVKFNWYVDGVEVHNAKTKPREEQYNSTYRVVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPG |
| | 140 | LC | DIQMTQTTSSLSASLGDRVTISCRTSQDIGNYLNWYQQKPDGTVKLLIYYTSRLHSGVPSRFSGSGSGT |
| | | | DFSLTINNVEQEDVATYFCQHYNTVPPTFGGGTKLEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| modo- | 141 | H-CDR1 | GYTFTSHW |
| tuximab | 142 | H-CDR2 | INPSSGRN |
| | 143 | H-CDR3 | VRYYGYDEAMDY |
| | 144 | L-CDR1 | KSLLHSNGITY |
| | 145 | L-CDR2 | QMS |
| | 146 | L-CDR3 | AQNLELPYT |
| | 147 | VH | QVQLQQPGAELVEPGGSVKLSCKASGYTFTSHWMHWVKQRPGQGLEWIGEINPSSGRNNYNEKFKSKAT |
| | | | LTVDKSSSTAYMQFSSLTSEDSAVYYCVRYYGYDEAMDYWGQGTSVTVSS |
| | 148 | VL | DIVMTQAAFSNPVTLGTSASISCRSSKSLLHSNGITYLYWYLQKPGQSPQLLIYQMSNLASGVPDRFSS |
| | | | SGSGTDFTLRISRVEAEDVGVYYCAQNLELPYTFGGGTKLEIK |
| | 149 | HC | QVQLQQPGAELVEPGGSVKLSCKASGYTFTSHWMHWVKQRPGQGLEWIGEINPSSGRNNYNEKFKSKAT |
| | | | LTVDKSSSTAYMQFSSLTSEDSAVYYCVRYYGYDEAMDYWGQGTSVTVSSASTKGPSVFPLAPSSKSTS |
| | | | GGTAALGCLVKDYFPEPVTVSWNSGALTSGVHTFPAVLQSSGLYSLSSVVTVPSSSLGTQTYICNVNHK |
| | | | PSNTKVDKRVEPKSCDKTHTCPPCPAPELLGGPSVFLFPPKPKDTLMISRTPEVTCVVVDVSHEDPEVK |
| | | | FNWYVDGVEVHNAKTKPREEQYNSTYRVVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPG |
| | 150 | LC | DIVMTQAAFSNPVTLGTSASISCRSSKSLLHSNGITYLYWYLQKPGQSPQLLIYQMSNLASGVPDRFSS |
| | | | SGSGTDFTLRISRVEAEDVGVYYCAQNLELPYTFGGGTKLEIKRTVAAPSVFIFPPSDEQLKSGTASVV |
| | | | CLLNNFYPREAKVQWKVDNALQSGNSQESVTEQDSKDSTYSLSSTLTLSKADYEKHKVYACEVTHQGLS |
| | | | SPVTKSFNRGEC |

HER2

| | | | |
|---|---|---|---|
| trastu- | 151 | H-CDR1 | GFNIKDTYI |
| zumab | 152 | H-CDR2 | YPTN |
| | 153 | H-CDR3 | GGDGFYAMD |
| | 154 | L-CDR1 | QDVNTAV |
| | 155 | L-CDR2 | AS |
| | 156 | L-CDR3 | HYTTPP |
| | 157 | VH | EVQLVESGGGLVQPGGSLRLSCAASGFNIKDTYIHWVRQAPGKGLEWVARIYPTNGYTRYADSVKGRFT |
| | | | ISADTSKNTAYLQMNSLRAEDTAVYYCSRWGGDGFYAMDYWGQGTLVTVSS |
| | 158 | VL | DIQMTQSPSSLSASVGDRVTITCRASQDVNTAVAWYQQKPGKAPKLLIYSASFLYSGVPSRFSGSRSGT |
| | | | DFTLTISSLQPEDFATYYCQQHYTTPPTFGQGTKVEIK |
| | 159 | HC | EVQLVESGGGLVQPGGSLRLSCAASGFNIKDTYIHWVRQAPGKGLEWVARIYPTNGYTRYADSVKGRFT |
| | | | ISADTSKNTAYLQMNSLRAEDTAVYYCSRWGGDGFYAMDYWGQGTLVTVSSASTKGPSVFPLAPSSKST |
| | | | SGGTAALGCLVKDYFPEPVTVSWNSGALTSGVHTFPAVLQSSGLYSLSSVVTVPSSSLGTQTYICNVNH |
| | | | KPSNTKVDKKVEPKSCDKTHTCPPCPAPELLGGPSVFLFPPKPKDTLMISRTPEVTCVVVDVSHEDPEV |
| | | | KFNWYVDGVEVHNAKTKPREEQYNSTYRVVSVLTVLHQDWLNGKEYKCKVSNKALPAPIEKTISKAKGQ |
| | | | PREPQVYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPG |
| | 160 | LC | DIQMTQSPSSLSASVGDRVTITCRASQDVNTAVAWYQQKPGKAPKLLIYSASFLYSGVPSRFSGSRSGT |
| | | | DFTLTISSLQPEDFATYYCQQHYTTPPTFGQGTKVEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |
| marge- | 161 | H-CDR1 | DTYIH |
| tuximab | 162 | H-CDR2 | RIYPTNGYTRYDPKFQD |
| | 163 | H-CDR3 | WGGDGFYAMDY |
| | 164 | L-CDR1 | KASQDVNTAVA |
| | 165 | L-CDR2 | SASFRYT |
| | 166 | L-CDR3 | QQHYTTPPT |
| | 167 | VH | QVQLQQSGPELVKPGASLKLSCTASGFNIKDTYIHWVKQRPEQGLEWIGRIYPTNGYTRYDPKFQDKAT |
| | | | ITADTSSNTAYLQVSRLTSEDTAVYYCSRWGGDGFYAMDYWGQGASVTVSS |
| | 168 | VL | DIVMTQSHKFMSTSVGDRVSITCKASQDVNTAVAWYQQKPGHSPKLLIYSASFRYTGVPDRFTGSRSGT |
| | | | DFTFTISSVQAEDLAVYYCQQHYTTPPTFGGGTKVEIK |
| | 169 | HC | QVQLQQSGPELVKPGASLKLSCTASGFNIKDTYIHWVKQRPEQGLEWIGRIYPTNGYTRYDPKFQDKAT |
| | | | ITADTSSNTAYLQVSRLTSEDTAVYYCSRWGGDGFYAMDYWGQGASVTVSSASTKGPSVFPLAPSSKST |
| | | | SGGTAALGCLVKDYFPEPVTVSWNSGALTSGVHTFPAVLQSSGLYSLSSVVTVPSSSLGTQTYICNVNH |
| | | | KPSNTKVDKRVEPKSCDKTHTCPPCPAPELVGGPSVFLLPPKPKDTLMISRTPEVTCVVVDVSHEDPEV |
| | | | KFNWYVDGVEVHNAKTKPPEEQYNSTLRVVSVLTVLHQDWLNGKEYKCKVSNKALPAPIEKTISKAKGQ |
| | | | PREPQVYTLPPSRDELTKNQVSLTCLVKGFYPSDIAVEWESNGQPENNYKTTPLVLDSDGSFFLYSKLT |
| | | | VDKSRWQQGNVFSCSVMHEALHNHYTQKSLSLSPGK |
| | 170 | LC | DIVMTQSHKFMSTSVGDRVSITCKASQDVNTAVAWYQQKPGHSPKLLIYSASFRYTGVPDRFTGSRSGT |
| | | | DFTFTISSVQAEDLAVYYCQQHYTTPPTFGGGTKVEIKRTVAAPSVFIFPPSDEQLKSGTASVVCLLNN |
| | | | FYPREAKVQWKVDNALQSGNSQESVTEQDSKDSTYSLSSTLTLSKADYEKHKVYACEVTHQGLSSPVTK |
| | | | SFNRGEC |

| | | |
|---|---|---|
| Human | 171 | MDNQGVIYSDLNLPPNPKRQQRKPKGNKNSILATEQEITYAELNLQKASQDFQGNDKTYHCKDLPSAPE |
| NKG2A | | KLIVGILGIICLILMASVVTIVVIPSTLIQRHNNSSLNTRTQKARHCGHCPEEWITYSNSCYYIGKERR |
| UniProt | | TWEESLLACTSKNSSLLSIDNEEEMKFLSIISPSSWIGVFRNSSHHPWVTMNGLAFKHEIKDSDNAELN |
| P26715 | | CAVLQVNRLKSAQCGSSIIYHCKHKL |
| Human | 172 | MQIPQAPWPVVWAVLQLGWRPGWFLDSPDRPWNPPTFSPALLVVTEGDNATFTCSFSNTSESFVLNWYR |
| PD-1 | | MSPSNQTDKLAAFPEDRSQPGQDCRFRVTQLPNGRDFHMSVVRARRNDSGTYLCGAISLAPKAQIKESL |
| UniProt | | RAELRVTERRAEVPTAHPSPSPRPAGQFQTLVVGVVGGLLGSLVLLVWVLAVICSRAARGTIGARRTGQ |
| Q15116 | | PLKEDPSAVPVFSVDYGELDFQWREKTPEPPVPCVPEQTEYATIVFPSGMGTSSPARRGSADGPRSAQP |
| | | LRPEDGHCSWPL |
| Human | 173 | MRIFAVFIFMTYWHLLNAFTVTVPKDLYVVEYGSNMTIECKFPVEKQLDLAALIVYWEMEDKNIIQFVH |
| PD-L1 | | GEEDLKVQHSSYRQRARLLKDQLSIGNAALQITDVKLQDAGVYRCMISYGGADYKRITVKVNAPYNKIN |
| UniProt | | QRILVVDPVTSEHELTCQAEGYPKAEVIWTSSDHQVLSGKTTTTNSKREEKLFNVTSTLRINTTTNEIF |
| Q9NZQ7 | | YCTFRRLDPEENHTAELVIPELPLAHPPNERTHLVILGAILLCLGVALTFIFRLRKGRMMDVKKCGIQD |
| | | TNSKKQSDTHLEET |
| Human | 174 | MRPSGTAGAALLALLAALCPASRALEEKKVCQGTSNKLTQLGTFEDHELSLQRMENNCEVVLGNLEITY |
| EGFR | | VQRNYDLSFLKTIQEVAGYVLIALNTVERIPLENLQIIRGNMYYENSYALAVLSNYDANKTGLKELPMR |
| UniProt | | NLQEILHGAVRFSNNPALCNVESIQWRDIVSSDFLSNMSMDFQNHLGSCQKCDPSCPNGSCWGAGEENC |
| P00533 | | QKLTKIICAQQCSGRCRGKSPSDCCHNQCAAGCTGPRESDCLVCRKFRDEATCKDTCPPLMLYNPTTYQ |
| | | MDVNPEGKYSFGATCVKKCPRNYVVTDHGSCVRACGADSYEMEEDGVRKCKKCEGPCRKVCNGIGIGEF |
| | | KDSLSINATNIKHFKNCTSISGDLHILPVAFRGDSFTHTPPLDPQELDILKTVKEITGELLIQAWPENR |
| | | TDLHAFENLEIIRGRTKQHGQFSLAVVSLNITSLGLRSLKEISDGDVIISGNKNLCYANTINWKKLEGT |
| | | SGQKTKIISNRGENSCKATGQVCHALCSPEGCWGPEPRDCVSCRNVSRGRECVDKCNLLEGEPREFVEN |
| | | SECIQCHPECLPQAMNITCTGRGPDNCIQCAHYIDGPHCVKTCPAGVMGENNTLVWKYADAGHVCHLCH |
| | | PNCTYGCTGPGLEGCPTNGPKIPSIATGMVGALLLLLVVALGIGLFMRRRHIVRKRTLRRLLQERELVE |
| | | PLTPSGEAPNQALLRILKETEFKKIKVLGSGAFGTVYKGLWIPEGEKVKIPVAIKELREATSPKANKEI |
| | | LDEAYVMASVDNPHVCRLIGICLTSTVQLITQLMPFGCLLDYVREHKDNIGSQYLLNWCVQIAKGMNYL |
| | | EDRRLVHRDLAARNVLVKTPQHVKITDFGLAKLLGAEEKEYHAEGGKVPIKWMALESILHRIYTHQSDV |
| | | WSYGVTVWELMTFGSKPYDGIPASEISSILEKGERLPQPPICTIDVYMIMVKCWMIDADSRPKFRELII |
| | | EFSKMARDPQRYLVIQGDERMHLPSPTDSNFYRALMDEEDMDDVVDADEYLIPQQGFFSSPSTSRTPLL |
| | | SSLSATSNNSTVACIDRNGLQSCPIKEDSFLQRYSSDPTGALTEDSIDDTFLPVPEYINQSVPKRPAGS |
| | | VQNPVYHNQPLNPAPSRDPHYQDPHSTAVGNPEYLNTVQPTCVNSTFDSPAHWAQKGSHQISLDNPDYQ |
| | | QDFFPKEAKPNGIFKGSTAENAEYLRVAPQSSEFIGA |
| Human | 175 | MELAALCRWGLLLALLPPGAASTQVCTGTDMKLRLPASPETHLDMLRHLYQGCQVVQGNLELTYLPTNA |
| HER2 | | SLSFLQDIQEVQGYVLIAHNQVRQVPLQRLRIVRGTQLFEDNYALAVLDNGDPLNNTTPVTGASPGGLR |
| UniProt | | ELQLRSLTEILKGGVLIQRNPQLCYQDTILWKDIFHKNNQLALTLIDTNRSRACHPCSPMCKGSRCWGE |
| P04626 | | SSEDCQSLTRTVCAGGCARCKGPLPTDCCHEQCAAGCTGPKHSDCLACLHFNHSGICELHCPALVTYNT |
| | | DTFESMPNPEGRYTFGASCVTACPYNYLSTDVGSCTLVCPLHNQEVTAEDGTQRCEKCSKPCARVCYGL |
| | | GMEHLREVRAVTSANIQEFAGCKKIFGSLAFLPESFDGDPASNTAPLQPEQLQVFETLEEITGYLYISA |
| | | WPDSLPDLSVFQNLQVIRGRILHNGAYSLTLQGLGISWLGLRSLRELGSGLALIHHNTHLCFVHTVPWD |
| | | QLFRNPHQALLHTANRPEDECVGEGLACHQLCARGHCWGPGPTQCVNCSQFLRGQECVEECRVLQGLPR |
| | | EYVNARHCLPCHPECQPQNGSVTCFGPEADQCVACAHYKDPPFCVARCPSGVKPDLSYMPIWKFPDEEG |
| | | ACQPCPINCTHSCVDLDDKGCPAEQRASPLTSIISAVVGILLVVVLGVVFGILIKRRQQKIRKYTMRRL |
| | | LQETELVEPLTPSGAMPNQAQMRILKETELRKVKVLGSGAFGTVYKGIWIPDGENVKIPVAIKVLRENT |
| | | SPKANKEILDEAYVMAGVGSPYVSRLLGICLTSTVQLVTQLMPYGCLLDHVRENRGRLGSQDLLNWCMQ |
| | | IAKGMSYLEDVRLVHRDLAARNVLVKSPNHVKITDFGLARLLDIDETEYHADGGKVPIKWMALESILRR |
| | | RFTHQSDVWSYGVTVWELMTFGAKPYDGIPAREIPDLLEKGERLPQPPICTIDVYMIMVKCWMIDSECR |
| | | PRFRELVSEFSRMARDPQRFVVIQNEDLGPASPLDSTFYRSLLEDDDMGDLVDAEEYLVPQQGFFCPDP |
| | | APGAGGMVHHRHRSSSTRSGGGDLTLGLEPSEEEAPRSPLAPSEGAGSDVEDGDLGMGAAKGLQSLPTH |
| | | DPSPLQRYSEDPTVPLPSETDGYVAPLTCSPQPEYVNQPDVRPQPPSPREGPLPAARPAGATLERPKTL |
| | | SPGKNGVVKDVFAFGGAVENPEYLTPQGGAAPQPHPPPAFSPAFDNLYYWDQDPPERGAPPSTFKGTPT |
| | | AENPEYLGLDVPV |
| (Gly4- | 176 | GGGGSGGGGSGGGGS |
| Ser)3 | | |
| Linker | | |
| Sequence | | |

1-74. (canceled)

75. A method of enhancing immunity in a human patient in need thereof, comprising administering to the patient
a) an anti-NKG2A antibody or an antigen-binding portion thereof that competes or cross-competes for binding to human NKG2A with, or binds to the same epitope of human NKG2A as, an antibody that comprises the heavy and light chain amino acid sequences of SEQ ID NOs: 9 and 10, respectively, or SEQ ID NOs: 19 and 20, respectively;
b) an anti-PD-1 antibody or an anti-PD-L1 antibody, or an antigen-binding portion thereof;
c) an anti-EGFR antibody component comprising one or two anti-EGFR antibodies or antigen-binding portions thereof, or an anti-HER2 antibody or an antigen-binding portion thereof;
d) a) and b); or
e) a), b), and c).

76. The method of claim 75, wherein the anti-NKG2A antibody comprises
a) heavy chain complementarity-determining regions (H-CDR) 1-3 and light chain complementarity-determining regions (L-CDR) 1-3 comprising the amino acid sequences of:
i) SEQ ID NOs: 1-6, respectively; or
ii) SEQ ID NOs: 11-16, respectively;
b) a heavy chain variable domain (VH) and a light chain variable domain (VL) comprising the amino acid sequences of:
i) SEQ ID NOs: 7 and 8, respectively; or
ii) SEQ ID NOs: 17 and 18, respectively; or
c) a heavy chain (HC) and a light chain (LC) comprising the amino acid sequences of:
i) SEQ ID NOs: 9 and 10, respectively; or
ii) SEQ ID NOs: 19 and 20, respectively.

77. The method of claim 75, wherein the anti-PD-1 antibody
a) comprises H-CDR1-3 and L-CDR1-3 comprising the amino acid sequences of:
i) SEQ ID NOs: 21-26, respectively;
ii) SEQ ID NOs: 31-36, respectively;
iii) SEQ ID NOs: 41-46, respectively;
iv) SEQ ID NOs: 51-56, respectively;
v) SEQ ID NOs: 61-66, respectively; or
vi) SEQ ID NOs: 71-76, respectively;
b) comprises a VH and a VL comprising the amino acid sequences of:
i) SEQ ID NOs: 27 and 28, respectively;
ii) SEQ ID NOs: 37 and 38, respectively;
iii) SEQ ID NOs: 47 and 48, respectively;
iv) SEQ ID NOs: 57 and 58, respectively;
v) SEQ ID NOs: 67 and 68, respectively; or
vi) SEQ ID NOs: 77 and 78, respectively;
c) comprises an HC and an LC comprising the amino acid sequences of:
i) SEQ ID NOs: 29 and 30, respectively;
ii) SEQ ID NOs: 39 and 40, respectively;
iii) SEQ ID NOs: 49 and 50, respectively;
iv) SEQ ID NOs: 59 and 60, respectively;
v) SEQ ID NOs: 69 and 70, respectively; or
vi) SEQ ID NOs: 79 and 80, respectively; or
d) is nivolumab, pembrolizumab, cemiplimab, dostarlimab, or retifanlimab.

78. The method of claim 75, wherein the anti-PD-L1 antibody
a) comprises H-CDR1-3 and L-CDR1-3 comprising the amino acid sequences of:
i) SEQ ID NOs: 81-86, respectively;
ii) SEQ ID NOs: 91-96, respectively; or
iii) SEQ ID NOs: 101-106, respectively;
b) comprises VH and VL comprising the amino acid sequences of:
i) SEQ ID NOs: 87 and 88, respectively;
ii) SEQ ID NOs: 97 and 98, respectively; or
iii) SEQ ID NOs: 107 and 108, respectively;
c) comprises HC and LC comprising the amino acid sequences of:
i) SEQ ID NOs: 89 and 90, respectively;
ii) SEQ ID NOs: 99 and 100, respectively; or
iii) SEQ ID NOs: 109 and 110, respectively; or
d) is atezolizumab, avelumab, or durvalumab.

79. The method of claim 75, wherein the anti-EGFR component
a) comprises an anti-EGFR antibody or an antigen-binding portion thereof with H-CDR1-3 and L-CDR1-3 that comprise the amino acid sequences of:
i) SEQ ID NOs: 111-116, respectively;
ii) SEQ ID NOs: 121-126, respectively;
iii) SEQ ID NOs: 131-136, respectively; or
iv) SEQ ID NOs: 141-146, respectively;
b) comprises an anti-EGFR antibody or an antigen-binding portion thereof with a VH and a VL that comprise the amino acid sequences of:
i) SEQ ID NOs: 117 and 118, respectively;
ii) SEQ ID NOs: 127 and 128, respectively;
iii) SEQ ID NOs: 137 and 138, respectively; or
iv) SEQ ID NOs: 147 and 148, respectively;
c) comprises an anti-EGFR antibody with an HC and an LC that comprise the amino acid sequences of:
i) SEQ ID NOs: 119 and 120, respectively;
ii) SEQ ID NOs: 129 and 130, respectively;
iii) SEQ ID NOs: 139 and 140, respectively; or
iv) SEQ ID NOs: 149 and 150, respectively;
d) comprises an anti-EGFR antibody or an antigen-binding portion thereof with H-CDR1-3 and L-CDR1-3 that comprise the amino acid sequences of SEQ ID NOs: 131-136, respectively, and an anti-EGFR antibody or an antigen-binding portion thereof with H-CDR1-3 and L-CDR1-3 that comprise the amino acid sequences of SEQ ID NOs: 141-146, respectively;
e) comprises an anti-EGFR antibody or an antigen-binding portion thereof with a VH and a VL that comprise the amino acid sequences of SEQ ID NOs: 137 and 138, respectively, and an anti-EGFR antibody or an antigen-binding portion thereof with a VH and VL that comprise the amino acid sequences of SEQ ID NOs: 147 and 148, respectively;
f) comprises an anti-EGFR antibody with an HC and an LC that comprise the amino acid sequences of SEQ ID NOs: 139 and 140, respectively, and an anti-EGFR antibody with an HC and LC that comprise the amino acid sequences of SEQ ID NOs: 149 and 150, respectively; or
g) is cetuximab, panitumumab, futuximab, modotuximab, or futuximab+modotuximab.

80. The method of claim 75, wherein the anti-HER2 antibody
a) comprises H-CDR1-3 and L-CDR1-3 comprising the amino acid sequences of:
i) SEQ ID NOs: 151-156, respectively; or
ii) SEQ ID NOs: 161-166, respectively;
b) comprises a VH and a VL comprising the amino acid sequences of:
i) SEQ ID NOs: 157 and 158, respectively; or
ii) SEQ ID NOs: 167 and 168, respectively;
c) comprises an HC and an LC comprising the amino acid sequences of:
i) SEQ ID NOs: 159 and 160, respectively; or
ii) SEQ ID NOs: 169 and 170, respectively;
d) is the anti-HER2 antibody of any of a)-c) conjugated to DXd or DM1; or
e) is trastuzumab, margetuximab, trastuzumab dexrutecan, or trastuzumab emtansine.

81. The method of claim 75, comprising administering to the patient:
a) an anti-NKG2A antibody or an antigen-binding portion thereof comprising the H-CDR1-3 and L-CDR1-3 amino acid sequences of SEQ ID NOs: 1-6, respectively; and an anti-PD-1 antibody or an antigen-binding portion thereof comprising the H-CDR1-3 and L-CDR1-3 amino acid sequences of SEQ ID NOs: 31-36, respectively;
b) an anti-NKG2A antibody or an antigen-binding portion thereof comprising the VH and VL amino acid sequences of SEQ ID NOs: 7 and 8, respectively; and an anti-PD-1 antibody or an antigen-binding portion thereof comprising the VH and VL amino acid sequences of SEQ ID NOs: 37 and 38, respectively;
c) an anti-NKG2A antibody comprising the HC and LC amino acid sequences of SEQ ID NOs: 9 and 10, respectively; and an anti-PD-1 antibody comprising the HC and LC amino acid sequences of SEQ ID NOs: 39 and 40, respectively;
d) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 61-66, respectively; and an anti-EGFR component comprising an anti-EGFR antibody or an antigen-binding portion thereof comprising the H-CDR1-3 and L-CDR1-3 amino acid sequences of SEQ ID NOs: 131-136, respectively, and an anti-EGFR antibody or an antigen-binding portion thereof comprising the H-CDR1-3 and L-CDR1-3 amino acid sequences of SEQ ID NOs: 141-146, respectively;
e) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 67 and 68, respectively; and an anti-EGFR component comprising an anti-EGFR antibody or an antigen-binding portion thereof comprising the VH and VL amino acid sequences of SEQ ID NOs: 137 and 138, respectively, and an anti-EGFR antibody or an antigen-binding portion thereof comprising the VH and VL amino acid sequences of SEQ ID NOs: 147 and 148, respectively;
f) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 69 and 70, respectively; and an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively;
g) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 61-66, respectively; and an anti-HER2 antibody or an antigen-binding portion thereof comprising the H-CDR1-3 and L-CDR1-3 amino acid sequences of SEQ ID NOs: 161-166, respectively;
h) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 67 and 68, respectively; and an anti-HER2 antibody or an antigen-binding portion thereof comprising the VH and VL amino acid sequences of SEQ ID NOs: 167 and 168, respectively;
i) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 69 and 70, respectively; and an anti-HER2 antibody comprising the HC and LC amino acid sequences of SEQ ID NOs: 169 and 170, respectively;
j) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 71-76, respectively; and an anti-HER2 antibody or an antigen-binding portion thereof comprising the H-CDR1-3 and L-CDR1-3 amino acid sequences of SEQ ID NOs: 161-166, respectively;
k) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 77 and 78, respectively; and an anti-HER2 antibody or an antigen-binding portion thereof comprising the VH and VL amino acid sequences of SEQ ID NOs: 167 and 168, respectively;
l) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 79 and 80, respectively; and an anti-HER2 antibody comprising the HC and LC amino acid sequences of SEQ ID NOs: 169 and 170, respectively;
m) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-HER2 antibody or an antigen-binding portion thereof comprising the H-CDR1-3 and L-CDR1-3 amino acid sequences of SEQ ID NOs: 151-156, respectively;
n) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-HER2 antibody or an antigen-binding portion thereof comprising the VH and VL amino acid sequences of SEQ ID NOs: 157 and 158, respectively;
o) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-HER2 antibody comprising the HC and LC amino acid sequences of SEQ ID NOs: 159 and 160, respectively;
p) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-EGFR component comprising an anti-EGFR antibody or an antigen-binding portion thereof comprising the H-CDR1-3 and L-CDR1-3 amino acid sequences of SEQ ID NOs: 131-136, respectively, and an anti-EGFR antibody or an antigen-binding portion thereof comprising the H-CDR1-3 and L-CDR1-3 amino acid sequences of SEQ ID NOs: 141-146, respectively;
q) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-EGFR component comprising an anti-EGFR antibody or an antigen-binding portion thereof comprising the VH and VL amino acid sequences of SEQ ID NOs: 137 and 138, respectively, and an anti-EGFR antibody or an antigen-binding portion thereof comprising the VH and VL amino acid sequences of SEQ ID NOs: 147 and 148, respectively;
r) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively;
s) an anti-NKG2A antibody or an antigen-binding portion thereof comprising the H-CDR1-3 and L-CDR1-3 amino acid sequences of SEQ ID NOs: 1-6, respectively; an anti-PD-1 antibody or an antigen-binding portion thereof comprising the H-CDR1-3 and L-CDR1-3 amino acid sequences of SEQ ID NOs: 31-36, respectively; and an anti-EGFR antibody or an antigen-binding portion thereof comprising the H-CDR1-3 and L-CDR1-3 amino acid sequences of SEQ ID NOs: 111-116, respectively;
t) an anti-NKG2A antibody or an antigen-binding portion thereof comprising the VH and VL amino acid sequences of SEQ ID NOs: 7 and 8, respectively; an anti-PD-1 antibody or an antigen-binding portion thereof comprising the VH and VL amino acid sequences of SEQ ID NOs: 37 and 38, respectively; and an anti-EGFR antibody or an antigen-binding portion thereof comprising the VH and VL amino acid sequences of SEQ ID NOs: 117 and 118, respectively; or
u) an anti-NKG2A antibody comprising the HC and LC amino acid sequences of SEQ ID NOs: 9 and 10, respectively; an anti-PD-1 antibody comprising the HC and LC amino acid sequences of SEQ ID NOs: 39 and 40, respectively; and an anti-EGFR antibody comprising the HC and LC amino acid sequences of SEQ ID NOs: 119 and 120, respectively.

82. The method of claim 75, wherein the antibodies or antigen-binding portions are administered to the patient concurrently or sequentially.

83. The method of claim 75, wherein
a) the anti-NKG2A antibody or antigen-binding portion thereof is administered at a dose of 8, 20, 100, 300, 750, or 1500 mg;
b) the anti-PD-1 or anti-PD-L1 antibody, or antigen-binding portion thereof, is administered at a dose of 200 mg;
c) the anti-EGFR component is administered at a dose of 6 mg/kg, 9 mg/kg, or a loading dose of 9 mg/kg followed by 6 mg/kg;
d) the anti-HER2 antibody or antigen-binding portion thereof is administered at a dose of 15 mg/kg; or
e) any combination of a)-d).

84. The method of claim 83, wherein
a) the anti-NKG2A antibody or antigen-binding portion thereof is administered in a 21-day cycle or 28-day cycle;
b) the anti-PD-1 or anti-PD-L1 antibody, or antigen-binding portion thereof, is administered every two weeks or three weeks;
c) the anti-PD-1 or anti-PD-L1 antibody, or antigen-binding portion thereof, is administered concomitantly with the anti-NKG2A antibody or antigen-binding portion thereof or after one cycle of the anti-NKG2A antibody or antigen-binding portion thereof,
d) the anti-EGFR component is administered weekly or every two weeks;
e) the anti-HER2 antibody or antigen-binding portion thereof is administered every three weeks or every four weeks; or
f) any combination of a)-e).

85. The method of claim 75, wherein the antibodies or antigen-binding portions are formulated for intravenous infusion.

86. The method of claim 75, wherein the patient has cancer.

87. The method of claim 86, wherein the cancer is a hematological malignancy, a solid tumor, colorectal cancer, gastric cancer or gastroesophageal cancer.

88. The method of claim 86, comprising administering to the patient an anti-NKG2A antibody comprising the HC and LC amino acid sequences of SEQ ID NOs: 9 and 10, respectively, wherein
a) the cancer is an advanced solid tumor malignancy, and the anti-NKG2A antibody is administered at a dose of 8, 20, 100, 300, 750, or 1500 mg every two weeks;
b) the cancer is an advanced solid tumor malignancy, and the anti-NKG2A antibody is administered at a dose of 20, 100, 300, 750, or 1500 mg every three weeks, wherein the method also comprises administering to the patient an anti-PD-1 antibody comprising the HC and LC amino acid sequences of SEQ ID NOs: 39 and 40, respectively, wherein after completing a 21-day cycle of anti-NKG2A antibody administration, the anti-PD-1 antibody is administered at a dose of 200 mg every three weeks;
c) the cancer is metastatic HER2+ gastric cancer, wherein the method also comprises administering to the patient
i) an anti-PD-1 antibody comprising the HC and LC amino acid sequences of SEQ ID NOs: 39 and 40, respectively; and
ii) an anti-HER2 antibody comprising the HC and LC amino acid sequences of SEQ ID NOs: 159 and 160, respectively;
d) the cancer is metastatic HER2+ gastric cancer, and the anti-NKG2A antibody is administered at a dose of 20, 100, 300, 750, or 1500 mg every two weeks, wherein the method also comprises administering to the patient
i) an anti-PD-1 antibody comprising the HC and LC amino acid sequences of SEQ ID NOs: 39 and 40, respectively, wherein after completing a 21-day cycle of anti-NKG2A antibody administration, the anti-PD-1 antibody is administered at a dose of 200 mg every three weeks; and
ii) an anti-HER2 antibody comprising the HC and LC amino acid sequences of SEQ ID NOs: 159 and 160, respectively, wherein the anti-HER2 antibody is administered at 15 mg/kg every three or four weeks;
wherein the antibodies are administered via intravenous infusion.

89. The method of claim 75, wherein the cancer is metastatic colorectal cancer, wherein the method comprising administering to the patient:
a) an anti-NKG2A antibody comprising the HC and LC amino acid sequences of SEQ ID NOs: 9 and 10, respectively;
b) an anti-PD-1 antibody comprising the HC and LC amino acid sequences of SEQ ID NOs: 69 and 70, respectively; and
c) an anti-EGFR component comprising an anti-EGFR antibody comprising the HC and LC amino acid sequences of SEQ ID NOs: 139 and 140, respectively, and an anti-EGFR antibody comprising the HC and LC amino acid sequences of SEQ ID NOs: 149 and 150, respectively.

90. The method of claim 89, wherein
the anti-NKG2A antibody is administered at a dose of 20, 100, 300, 750, or 1500 mg every two weeks,
after completing a 28-day cycle of anti-NKG2A antibody administration, the anti-PD-1 antibody is administered at a dose of 200 mg every two weeks, and
the anti-EGFR component is administered at a loading dose of 9 mg/kg followed by a dose of 6 mg/kg every one or two weeks,
wherein the antibodies are administered via intravenous infusion.

91. The method of claim 89, wherein
a) the patient has low microsatellite instability status;
b) the patient is
i) without RAS mutations in any of the following codons: codons 12 and 13 in exon 2, codons 59 and 61 in exon 3, and codons 117 and 146 in exon 4;
ii) without the BRAF V600E mutation; or
iii) i) and ii); or
c) a) and b)

92. The method of claim 75, further comprising administering to the patient radiation therapy, a chemotherapeutic agent, an anti-neoplastic agent, an anti-angiogenic agent, or any combination thereof.

93. A multi-specific antibody that specifically binds to:
a) human NKG2A and human PD-1;
b) human NKG2A and human PD-L1;
c) human NKG2A, human PD-1, and human EGFR;
d) human NKG2A, human PD-1, and human HER2;
e) human NKG2A, human PD-L1, and human EGFR; or
f) human NKG2A, human PD-L1, and human HER2;
wherein the multi-specific antibody comprises an NKG2A binding domain that competes or cross-competes for binding to human NKG2A with, or binds to the same epitope of human NKG2A as, an antibody that comprises the heavy and light chain amino acid sequences of SEQ ID NOs: 9 and 10, respectively, or SEQ ID NOs: 19 and 20, respectively.

94. A pharmaceutical composition comprising an anti-NKG2A antibody or an antigen-binding portion thereof that competes or cross-competes for binding to human NKG2A with, or binds to the same epitope of human NKG2A as, an antibody that comprises the heavy and light chain amino acid sequences of SEQ ID NOs: 9 and 10, respectively, or SEQ ID NOs: 19 and 20, respectively, and further comprising:
a) an anti-PD-1 antibody or an antigen-binding portion thereof;
b) an anti-PD-L1 antibody or an antigen-binding portion thereof;
c) an anti-PD-1 antibody or an antigen-binding portion thereof and an anti-EGFR component;
d) an anti-PD-1 antibody or an antigen-binding portion thereof and an anti-HER2 antibody or an antigen-binding portion thereof;
e) an anti-PD-L1 antibody or an antigen-binding portion thereof and an anti-EGFR component; or
f) an anti-PD-L1 antibody or an antigen-binding portion thereof and an anti-HER2 antibody or an antigen-binding portion thereof,
and a pharmaceutically acceptable excipient.
